SEQUENCE LISTING (24 sequences)

SEQ ID NO: 1 - protein, 97 aa, synthetic construct
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 2 - DNA, 48 bp, synthetic construct
  source 1..48: mol_type = other DNA
ccatgatgtttacatttcacactcgagtgtgaaatgtaaacatcatgg

SEQ ID NO: 3 - DNA, 48 bp, synthetic construct
  source 1..48: mol_type = other DNA
ccattgcatgcaatgactattctcgagaatagtcattgcatgcaatgg

SEQ ID NO: 4 - DNA, 20 bp, synthetic construct
  source 1..20: mol_type = other DNA
tcgctggggataaaggctac

SEQ ID NO: 5 - DNA, 20 bp, synthetic construct
  source 1..20: mol_type = other DNA
aagaagctctggaagcccac

SEQ ID NO: 6 - DNA, 20 bp, synthetic construct
  source 1..20: mol_type = other DNA
gctcctacccacgcagattt

SEQ ID NO: 7 - DNA, 20 bp, synthetic construct
  source 1..20: mol_type = other DNA
gagaccacgaagagactggc

SEQ ID NO: 8 - DNA, 19 bp, synthetic construct
  source 1..19: mol_type = other DNA
tgccgctttgcaggtgtat

SEQ ID NO: 9 - DNA, 17 bp, synthetic construct
  source 1..17: mol_type = other DNA
ggcctccgtccgagaga

SEQ ID NO: 10 - DNA, 21 bp, synthetic construct
  source 1..21: mol_type = other DNA
cccgaatcccctgcccagtct

SEQ ID NO: 11 - DNA, 21 bp, synthetic construct
  source 1..21: mol_type = other DNA
tcatccagccacaggtgccta

SEQ ID NO: 12 - DNA, 25 bp, synthetic construct
  source 1..25: mol_type = other DNA
aaatgggaggggtgcaaaagaggag

SEQ ID NO: 13 - DNA, 25 bp, synthetic construct
  source 1..25: mol_type = other DNA
cagctgtcatttgctgtgggtgatg

SEQ ID NO: 14 - DNA, 20 bp, synthetic construct
  source 1..20: mol_type = other DNA
cgtcctcggattctctgctc

SEQ ID NO: 15 - DNA, 20 bp, synthetic construct
  source 1..20: mol_type = other DNA
gctggtgcattttcggttgt

SEQ ID NO: 16 - DNA, 23 bp, synthetic construct
  source 1..23: mol_type = other DNA
tcatcagcctcgatattccatct

SEQ ID NO: 17 - DNA, 21 bp, synthetic construct
  source 1..21: mol_type = other DNA
ggcccgtggaacataagtctt

SEQ ID NO: 18 - DNA, 21 bp, synthetic construct
  source 1..21: mol_type = other DNA
ggagcgagatccctccaaaat

SEQ ID NO: 19 - DNA, 23 bp, synthetic construct
  source 1..23: mol_type = other DNA
ggctgttgtcatacttctcatgg

SEQ ID NO: 20 - DNA, 21 bp, synthetic construct
  source 1..21: mol_type = other DNA
ccatgatgtttacatttcaca

SEQ ID NO: 21 - DNA, 21 bp, synthetic construct
  source 1..21: mol_type = other DNA
ccattgcatgcaatgactatt

SEQ ID NO: 22 - DNA, 20 bp, synthetic construct
  source 1..20: mol_type = other DNA
ggaggacctgagagtactca

SEQ ID NO: 23 - DNA, 20 bp, synthetic construct
  source 1..20: mol_type = other DNA
gtagtacacatgaagcagaa

SEQ ID NO: 24 - DNA, 21 bp, synthetic construct
  source 1..21: mol_type = other DNA
cactccaagaacatgactatt

(19) United States
(12) Patent Application Publication
(10) Pub. No.: US 2024/0094211 A1
(43) Pub. Date: Mar. 21, 2024

(54) COMPOSITIONS AND METHODS TARGETING S100A10 FOR THE TREATMENT AND DIAGNOSIS OF LIVER CANCER

(71) Applicant: The University of Hong Kong, Hong Kong (CN)

(72) Inventors: Irene Oi Lin Lui Ng, Hong Kong (CN); Xia Wang, Hong Kong (CN)

(21) Appl. No.: 18/352,040

(22) Filed: Jul. 13, 2023

Related U.S. Application Data
(60) Provisional application No. 63/390,453, filed on Jul. 19, 2022.

(51) Int. Cl. G01N 33/574; C12Q 1/6851; C12Q 1/6886

(52) U.S. Cl. CPC G01N 33/57438; C12Q 1/6851; C12Q 1/6886; G01N 33/57496; C12Q 2600/158; G01N 2333/4727

(57) ABSTRACT

Compositions and methods for early diagnosis, risk stratification, and effective management and treatment of patients with hepatocellular carcinoma are provided. The compositions and methods selectively target S100A10, for effective detection of S100A10 in a subject, for example, from plasma samples or from tissue biopsy, and for effective inhibiting and/or reducing the activities and/or quantities of S100A10 in vitro and/or in vivo. Methods for selecting patients who would be amenable for the disclosed therapies and for treating such patients are also described.

### CROSS REFERENCE TO RELATED APPLICATIONS

This application claims benefit of and priority to U.S. Provisional Application No. 63/390,453, filed Jul. 19, 2022, which is specifically incorporated by reference herein in its entirety.

### REFERENCE TO THE SEQUENCE LISTING

The Sequence Listing submitted as a text file named “UHK_01027_US_ST26.xml” created on Jul. 5, 2023, and having a size of 22,375 bytes is hereby incorporated by reference pursuant to 37 C.F.R. § 1.834(c)(1).

### FIELD OF THE INVENTION

The invention is generally directed to the use of S100A10 as a diagnostic marker and therapeutic target in liver cancer.

### BACKGROUND OF THE INVENTION

Hepatocellular carcinoma (HCC) is the fourth most common cause of cancer-related death worldwide. HCC is the second and third leading cause of cancer deaths in China and Hong Kong, respectively. Risk factors for HCC include chronic hepatitis B and hepatitis C, alcohol addiction, metabolic liver disease (particularly nonalcoholic fatty liver disease) and exposure to dietary toxins such as aflatoxins and aristolochic acid. The 5-year survival rate of HCC patients, even after surgical removal of the cancer, is only ˜20%. The median survival rates of patients with inoperable HCC are only in weeks. Its high mortality rate is attributable to its aggressive behavior and lack of promising curative therapy. New treatment modalities for this cancer are much awaited.

HCC surveillance and early detection increase the chance of potentially curative treatment. However, HCC is normally asymptomatic in the early stages and has a high propensity for intravascular or intractable invasion, even when the primary tumor is small. As a result, most patients have incurable disease at the time of diagnosis. Thus, new molecular markers are urgently needed for early detection of HCC.

Therefore, it is an object of the invention to provide a diagnostic biomarker for early diagnosis and tumor recurrence of patients with liver cancer such as HCC.

It is another object to provide compositions and methods for the treatment of liver cancer.

### SUMMARY OF THE INVENTION

Compositions and methods for identifying a subject as having an elevated risk of having hepatocellular carcinoma (HCC) are provided. The methods include the steps of i) obtaining a sample from the subject; ii) determining that the level of S100A10 in the sample compared to a control; iii) diagnosing the subject as having HCC if the level of S100A10 in the sample is increased compared to a control such as a healthy subject without HCC. Typically, the sample is a plasma sample or a liver biopsy. In some forms, the subject diagnosed as having HCC has a level of S100A10 is at least 10%, 20%, 30%, 40%, 50%, 60%, 70%, 80%, 90%, 100%, or more than 100% greater than the control. In some forms, the step of determining that the level of S100A10 in the sample includes measuring the level of S100A10 protein in the sample, preferably the level of S100A10 protein derived from the extracellular vesicles of the sample, for example, by performing an enzyme-linked immunosorbent assay (ELISA), radioimmunoassay (RIA), western blot, or dot blot. In other forms, the step of determining that the level of S100A10 in the sample includes measuring the level of S100A10 mRNA in the sample, for example, by performing a hybridization assay, Real-time Polymerase chain reaction (RT-PCR), or Quantitative Polymerase chain reaction (qPCR). Preferably, the diagnosis of the subject as having HCC is with at least a 50%, 60%, 70%, 80%, 90%, 95%, 97%, or 99% certainty. In preferred forms, the method further includes the step of administering a therapeutic agent for treating HCC. Typically, the therapeutic agents are small molecules effective in inhibiting the S100A10-annexin A2 interaction, neutralizing antibodies against annexin A2 and/or S100A10, or inhibitory nucleic acids such as shRNA targeting S100A10.

Methods of treating a subject having hepatocellular carcinoma (HCC) are also provided. The methods include the step of administering to the subject an effective amount of a therapeutic agent for treating HCC. Typically, the therapeutic agents are small molecules effective in inhibiting the S100A10-annexin A2 interaction, neutralizing antibodies against annexin A2 and/or S100A10, or inhibitory nucleic acids such as shRNA targeting S100A10. Methods include the step of selecting patients who would be amenable for therapies that inhibit and/or reduce the activities and/or quantities of S100A10. In some form, the subject receiving treatment has an elevated level of S100A10 compared to a healthy control, for example, at least 10%, 20%, 30%, 40%, 50%, 60%, 70%, 80%, 90%, 100%, or more than 100% greater than the control. In preferred forms, the therapeutic agent is administered in an amount effective to reduce or inhibit proliferation, migration, invasion, motility, and/or metastatic abilities of the cancer cells of HCC. In further preferred forms, the therapeutic agent is administered in an amount effective to reduce or inhibit tumor growth, tumor burden, and/or increase survival of the subject. In other forms, the therapeutic agent is administered in an amount effective to reduce or inhibit the number of cancer stem cells (CSCs) in HCC, or the number of the cancer cells expressing one or more of CD24, CD44, LGR5, SOX2, c-MYC, CD133, EpCAM, and ABCG2, or the level of expression of one or more of CD24, CD44, LGR5, SOX2, c-MYC, CD133, EpCAM, and ABCG2 on the cancer cells. In yet further forms, the therapeutic agent is administered in an amount effective to reduce or inhibit activities and/or quantities of one or more kinases of EGFR, AKT and ERK signaling associated with epithelial-mesenchymal transition of the cancer cells.

### BRIEF DESCRIPTION OF THE DRAWINGS

FIGS. 1A-1G show the clinical significance of S100A10. FIG. 1A is a schematic showing the induction of Human ES cells to differentiate along hepatic lineages into adult hepatocytes. Cells from different developmental stages, including ES cells, DE, LP cells and PHs, as well as normal liver (NL) and HCC tissues, were used for transcriptomic sequencing. The expression pattern of S100A10 was confirmed using qRT-PCR. FIG. 1B is a scatterplot showing the relative expression of S100A10 in HCC and corresponding non-tumorous livers from 86 pairs of samples of patients with HCC. Paired t-test. FIG. 1C is a Kaplan-Meier overall survival curve and numbers at risk for these 86 patients with HCC. FIGS. 1D and 1E show the relative S100A10 expression (FIG. 1D) and Kaplan-Meier curve (FIG. 1E) for overall survival of TCGA cohort (analysed by GEPIA website). FIG. 1F is a bar graph showing CNV of S100A10 in 80 pairs of samples of patients with HCC. FIG. 1G shows S100A10 expression stratified according to S100A10 CNV (left panel), non-parametric Mann-Whitney test. *P<0.05, **P<0.01. Correlation between relative S100A10 expression and relative CNV (right panel). AJCC, American Joint Committee on Cancer; CNV, copy number variation; DE, definitive endoderm; ES, embryonic stem; HCC, hepatocellular carcinoma; LIHC, liver hepatocellular carcinoma; LP, liver progenitor; NT, non-tumorous liver tissues; TCGA, The Cancer Genome Atlas; TNM, TNM classification of malignant tumors.

FIGS. 2A-2J show that S100A10 enhances HCC sternness. FIG. 2A is a western blot showing efficiency of ectopic S100A10 expression, and S100A10 silencing with shRNA against S100A10 (shS100A10-1: sh1, shS100A10-3: sh3) and with sgRNA against S100A10 (sgS100A10-2: sg2, sgS100A10-4: sg4). S100, S100A10; NTC, non-target control. Tubulin was used as a loading control. Band intensities were quantitated by ImageJ. FIG. 2B shows relative expression of HCC stemness-related markers with S100A10-OE compared with Vec plasmid by qRT-PCR. FIGS. 2C-2E are bar graphs showing sphere formation assay of PLC-Vec/S100A10, 97L-Vec/S100A10 and 97H-NTC/sgS100A10s, respectively. FIG. 2F is a limiting dilution assays of PLC-Vec/S100A10. The tumor-initiating frequency is summarized in chart and table form in Table 2. FIGS. 2G-2I show chemoresistance assay in nude mice by subcutaneous injection. Representative images of PLC-Vec/S100A10 tumors treated with indicated drugs. Tumour weights expressed as mean±SD of five mice in each group, Mann-Whitney test (lower panel). FIG. 2J is a western blot showing the expression of p-AKT, AKT, p-ERK and ERK in PLC-Vec/S100A10, 97L-Vec/S100A10 and 97H-NTC/sgS100A10s, respectively. Tubulin was used as a loading control. HCC, hepatocellular carcinoma; NTC, non-target control; p-AKT, phosphorylated AKT; p-ERK, phosphorylated ERK; Vec, vector control.

FIGS. 3A-3K show that S100A10 promotes HCC migratory, invasive and metastatic abilities in vitro and in vivo. FIGS. 3A-3G show the migratory and invasive abilities of PLC-Vec/S100A10, 97L-Vec/S100A10 and Huh7-NTC/shS100A10 were assessed by transwell migration and matrigel invasion assays. FIGS. 3H and 3I are bar charts showing the numbers of metastatic nodules on the liver surface (yellow arrows) in six mice in each group. FIG. 3J shows the bioluminescence signals represent mean±SD. *P<0.05, **P<0.01. Mann-Whitney test calculated from images of lungs from NOD SCID mice after tail vein injection of 97L-Vec/S100A10 and representative histology of the corresponding lung sections. FIG. 3K is a western blot showing the expression of E-cadherin, N-cadherin, vimentin and fibronectin of HCC cells transfected with Vec and S100A10, or NTC and sgS100A10s. HCC, hepatocellular carcinoma; NTC, non-target control; Vec, vector control.

FIGS. 4A-4J show S100A10 is present in HCC-derived EVs and promotes HCC motility and metastasis. FIG. 4A is a western blot showing detection of S100A10 and EV markers including CD81, CD63, HSP70, CD9, TSG101, Alix, Golgi marker GM130 and nucleoporin p62 in EVs from the plasma of patients with HCC. Each lane represents either a healthy donor or a patient with HCC. FIG. 4B is a western blot showing detection of S100A10 and EV markers including CD81, CD63, HSP70, CD9, TSG101, Alix and Golgi marker GM130 and nucleoporin p62 in EVs from HCC cells. FIG. 4C are representative electron-micrographs of EVs derived from the plasma of patients with HCC, 97H and 97L cells (upper panel) Immunogold labelling of EVs using anti-CD63 and anti-S100A10 antibodies followed by secondary antibodies conjugated to 5 and 10 nm gold particles, respectively (lower panel). FIGS. 4D-4G are bar graphs of cell migration and invasion assays of PLC cells treated with Vec EVs or S100A10-OE cells (S100 EVs), or with EVs from 97H-NTC (NTC EVs) or -sgS100A10 #4 (sgS100 EVs) cells. FIG. 4H is a schematic diagram of the two EV education mouse models: liver metastasis model (left panel) and lung metastasis model (right panel). FIGS. 4I and 4J are graphs the numbers of metastatic nodules on liver surface and bioluminescence signals of lung are summarized in the bar charts (right panel). EV, extracellular vesicle; HCC, hepatocellular carcinoma; NTC, non-target control; Vec, vector control; Vec EV, extracellular vesicle from vector control cell.

FIGS. 5A-5G show S100A10-NA abrogates the functions of S100A10 EVs in HCC stemness. FIGS. 5A and 5B are bar graphs showing migration and invasion assays on PLC cells treated with PBS or EVs derived from S100A10-OE cells (S100 EVs). The EVs were preincubated and added together with S100A10 NA or IgG control antibody. FIG. 5C shows intrasplenic injection of HCC cells. EVs were injected together with S100A10 NA or IgG. FIG. 5D shows bioluminescence signals of lungs from NOD SCID mice after tail vein injection of luciferase labelled 97L, educated with PBS or indicated EVs together with S100A10 NA or IgG. Sphere formation (FIG. 5E) and chemoresistance (FIG. 5F) were assessed on PLC cells treated with PBS, Vec EVs, or EVs from S100A10-OE cells (S100 EVs). The EVs were preincubated and treated together with S100A10 NA or IgG control antibody. FIG. 5G shows quantification of lung vascular leakiness after tail vein injection of EVs, Texas Red-Dextran and FITC-lectin. Arrowheads indicate the area of endothelial leakiness. Scale bar=20 μm. *P<0.05, **P<0.01. EV, extracellular vesicle; NA, neutralising antibody; ns, not significant; Vec, vector control; Vec EV, extracellular vesicle from vector control cell.

FIGS. 6A-6G show that S100A10 alters the protein contents of EVs. FIG. 6A is a Western blot showing the levels of MMP2, EGF, fibronectin and ITGAV in EVs derived from PLC-Vec/S100A10 and 97L-Vec/S100A10. FIG. 6B is a Western blot showing the expression levels of S100A10, MMP2, EGF, fibronectin and ITGAV in EVs derived from 97H-NTC/sgS100A10s or 97 H cells treated with S100A10 NA or IgG 48 hours before EV isolation. The EV markers, including CD63, CD81, HSP70, TSG101 and Alix, were used as loading control. FIG. 6C is a Representative electron micrograph of EVs from the plasma of patients with HCC subjected to immunogold labelling using anti-S100A10 together with anti-MMP2, anti-EGF, anti-fibronectin or anti-ITGAV antibodies, followed by secondary antibodies conjugated to 10 and 5 nm gold particles, respectively. Scale bar=25 nm. FIG. 6D is a Western blot showing the expression of S100A10, MMP2, EGF, fibronectin, surface marker CD81 and intra-EV marker HSP70 in 97H-derived EVs treated with increasing concentrations of proteinase K. Untreated EVs (first lane from left) served as control. FIG. 6E is a Western blot showing MMP2, EGF, fibronectin, S100A10 and ITGAV in EVs. GRADSP peptide (control peptide RAD as control) or GRGDSP peptide (RGD which is integrin-binding) were added to PLC-S100A10 cells for 48 hours before EV isolation. HSP70 was used as a loading control. FIG. 6F is a Coimmunoprecipitation assay showing the interaction between S100A10 and MMP2, EGF, fibronectin and ITGAV, using anti-S100A10 (IP-S100A10) or IgG (IP-IgG) in S100A10-OE PLC cells. Total cell lysate (input) used as positive control. FIG. 6G is a schematic diagram illustrating the proposed S100A10-mediated binding of MMP2, fibronectin and EGF to the surface of EVs through ITGAV. EV, extracellular vesicle; HCC, hepatocellular carcinoma; ITGAV, integrin αV; NA, neutralising antibody; NTC, non-target control.

FIGS. 7A-7G show EV-S100A10 and S100A10 enhance HCC progression through EGFR activation. FIG. 7A is a Western blot showing the expression levels of EMT-related markers and EGFR, AKT and ERK signalling in cells treated with PBS, Vec EVs, 5100 EVs, or 5100 EVs, together with S100A10 NA or IgG. FIG. 7B is a Western blot showing the activation of EGFR, AKT and ERK signalling in cells treated with PBS, Vec EVs, 5100 EVs, or 5100 EVs together with different concentrations of EGFR inhibitor-Gefi (μM). FIGS. 7C and 7D show Chemoresistance to sorafenib of PLC-Vec, S100A10-OE cells, or S100A10-OE cells treated with Gefi, S100A10 NA or IgG control antibody. FIG. 7E is a Western blot showing the activation of EGFR, AKT and ERK signalling in Vec or S100A10-OE cells treated with S100A10 NA, IgG or different concentrations of Gefi (μM). FIGS. 7F and 7G shows quantification from experiments in which nude mice were subcutaneously injected with S100A10-OE PLC cells were administered with vehicle and IgG, sorafenib, S100A10 NA, or a combination of sorafenib and S100A10 NA. Sorafenib was administered daily via oral gavage. Control IgG and S100A10 NA were injected peritoneally once every 3 days. The tumour weight (FIG. 7F) and body weight (FIG. 7G) of mice were measured and plotted (right panel). *P<0.05, **P<0.01. EV, extracellular vesicle; Gefi, gefitinib; Gefi-5, gefitinib at 5 μM; p-AKT, phosphorylated AKT; p-ERK, phosphorylated ERK; Vec, vector control.

FIGS. 8A-8I show EV-S100A10 promotes chemotaxis of HCC cells. FIGS. 8A-8D are Cell migration and invasion assays. Vec EVs, or S100A10-OE cells (S100 EVs), or 97H-NTC (NTC EVs) or -sgS100A10 #4 (sgS100 EVs) were added to the lower compartment of transwell chambers as indicated. FIGS. 8E and 8F show migratory and invasive abilities of PLC cells assessed with PBS or S100 EVs added to the lower compartment of transwell chambers. EVs were preincubated and added together with S100A10 NA or IgG antibody. FIG. 8G show Migratory ability of PLC treated with IgG or EGFR NA, with S100A10 EVs and IgG or S100A10 NA were added to the lower compartment of transwell chambers. FIG. 8H show Migratory ability of PLC treated with IgG or EGFR NA, with CM and IgG or S100A10 NA were added to the lower compartment of transwell chambers. FIG. 8I shows coimmunoprecipitation assay showing interaction between S100A10 and EGFR using anti-S100A10 (IP-S100A10) or anti-EGFR antibody (IP-EGFR) or IgG (IP-IgG) in S100A10-OE PLC cells. Total cell lysate (input) used as positive control. EV, extracellular vesicle; NTC, non-target control; Vec, vector control; Vec EV, extracellular vesicle from vector control cell.

FIG. 9 is a bar graph showing the copy number variation (CNV) of S100A10 detected by TaqMan Copy Number Assay in paired HCC and corresponding nontumorous liver tissues (NT).

FIGS. 10A-10J show S100A10 promotes the tumorigenicity of HCC. FIG. 10A shows the relative mRNA expression of HCC stemness-related markers of CD24, CD44, LGR5, SOX2, c-MYC and ABCG2 in 97L-5100A10-OE as compared with Vec control using qPT-PCR. FIGS. 10B-10F show focus formation assays of S100A10/Vec, shS100A10s/NTC and sgS100A10s/NTC in PLC, 97L, MIHA, Huh7 and 97H cells, respectively. FIG. 10G shows sphere formation ability induced by MIHA-S100A10. FIG. 10H shows limiting dilution assays of 97L-Vec/S100A10. The tumor-initiating frequencies are summarized in chart and table form. FIGS. 10I and 10J shows Subcutaneous tumors formed by 97H-sgS100A10 #4 and Huh7-shS100A10 #3 were much smaller than their NTC counterparts; Mean±SD of 5-6 mice.

FIGS. 11A-11M show S100A10 enhances chemoresistance of HCC. FIGS. 11A-11I show XTT assay showing chemoresistance of PLC-Vec/S100A10, 97L-Vec/S100A10 and 97H-NTC/sgS100A10s to sorafenib, cisplatin and 5-FU. FIGS. 11J-11L show apoptosis analysis using annexin-V staining and flow cytometry on PLC-Vec/S100A10, 97L-Vec/S100A10 and 97HBMJ NTC/sgS100A10s treated with sorafenib, cisplatin and 5-FU, respectively. FIG. 11M shows relative S100A10 expression detected by qRT-PCR under treatment as indicated (left panel): Sora-4, Sora-8: sorafenib at 4 or 8 μM; CDDP-4, CDDP-8: cisplatin at 4 or 8 μg/mL; 5-FU-50, 5-FU-100: 5-FU at 50 or 100 μg/mL. S100A10 and HIF-1α expression detected by western blot (right panel). Tubulin was used as a loading control.

FIGS. 12A-12H show S100A10 is present in HCC-derived EVs and promotes HCC motility. FIGS. 12A-12D show size distribution of EVs from HCC patient's plasma, 97H, 97L, and PLC cells, as measured by ZetaView Particle Tracking Analyzer. FIG. 12E is a Western blot showing detection of S100A10 and EVs markers including CD63, HSP70, and Golgi marker GM130 in EVs from HCC patients' plasma. Each lane represents either a healthy donor or a HCC patient. FIG. 12F shows the levels of S100A10 in EVs from all clinical samples were quantified and normalized to the average of HSP70 and CD63 (lower panel). FIG. 12G is Representative electron micrograph showing the morphology of EVs from PLC and Huh7 cells. Scale bar=100 nm. FIG. 12H is a cell migration assay of PLC cells treated with EVs from S100A10-OE cells (S100 EVs) at indicated concentrations.

FIGS. 13A-13E show S100A10 EVs promote HCC motility and metastasis. FIGS. 13A-13D show Cell migration and invasion assays of 97L cells treated with EVs from the corresponding vector control cells (Vec EVs) or S100A10-OE cells (S100 EVs), or with EVs from 97H-NTC (NTC EVs) or -sgS100A10 #4 (sgS100 EVs) cells. FIG. 13E shows number livers in the intrasplenic injection model of 97H cells in nude mice educated with PBS or indicated EVs.

FIGS. 14A-14E show S100A10 in EVs plays a key role in inducing HCC sternness features. FIGS. 14A-14C show Focus Formation and chemosresistance as assessed on PLC cells treated with PBS, EVs from vector control cells (Vec EVs), or from S100A10-OE cells (S100 EVs). The EVs were preincubated and treated together with S100A10 NA or IgG control antibody. FIGS. 14D and 14E show quantification of the distribution of EVs derived from 97L-Vec or S100A10-OE in livers and lungs of mice. The mice were sacrificed 24 h after intravenously injected with EVs labeled with PKH26. Tissues were subjected to frozen sections and examined under confocal microscopy. PKH26+EVs as seen in red color are indicated by the arrowhead. DAPI (blue) was used for nuclei counterstaining. Quantification of the percentage of PKH26+ cells in three random fields of three tissue sections per organ is shown.

FIGS. 15A-15D shows S100A10 alters the protein content of EVs. FIGS. 15A and 15B show Mass spectrometry (MS) was performed to compare the different proteins in 97L-Vec-EVs and 97L-S100A10-EVs. Gene ontology (GO) analysis and KEGG analysis were used to identify the molecular functions and signaling pathways enriched in S100A10 upregulated proteins in EVs. EVs derived from PLC-Vec/S100A10 or 97H-NTC/sgS100A10s were subject to zymography assay to measure the MMP-2 activity. 20 ng of recombinant MMP-2 served as positive controls. FIG. 15C shows quantification from a migration assay of PLC cells treated with S100 EVs with or without MMP2 inhibitor (MMP2i). FIG. 15D is a Western blot showing the expression levels of S100A10, MMP2, EGF, fibronectin and ITGAV in 97H-NTC/sgS100A10s or 97H cells treated with S100A10 NA or IgG.

FIGS. 16A-16E show EGFR inhibitor Gefitinib suppresses the oncogenic function of S100A10. FIG. 16A shows XTT assay on PLC-Vec/S100A10 treated with different concentrations of Gefitinib. FIG. 16B shows focus formation. FIG. 16C shows number of spheres formed. FIGS. 16D and 16E show number of migrated cells and invaded cells of PLC-Vec/S100A10 treated with Gefitinib as indicated. Gefi-1, Gefi-5: Gefitinib at 1 or 5 μM.

FIGS. 17A-17L shows EV-S100A10 promotes chemotaxis of HCC cells. FIGS. 17A-17H shows PLC and 97L migration and invasion assays using conditional medium (CM) derived from corresponding Vec (Vec CM), S100A10-OE (S100A10 CM), or 97H-NTC (NTC CM)/-sgS100A10 #4 (sgS100A10 CM) in the lower compartment of transwell. FIGS. 17I-17L show Migration and invasion assays on 97L cells. EVs derived from 97L-Vec (Vec EVs)/S100A10 (S100 EVs), or 97H-NTC (NTC EVs)/-sgS100A10 #4 (sgS100 EVs) were added to the lower compartment of transwell chambers as indicated.

### DETAILED DESCRIPTION OF THE INVENTION

I. Definitions

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” means a dosage sufficient to treat, inhibit, or alleviate one or more symptoms of a disease state being treated or to otherwise provide a desired pharmacologic and/or physiologic effect. In some forms, the effective amount refers to the amount which is able to treat one or more symptoms of hepatocellular carcinoma (HCC), reverse the progression of one or more symptoms of HCC, halt the progression of one or more symptoms of HCC, or prevent the occurrence of one or more symptoms of HCC in a subject to whom the formulation is administered, for example, as compared to a matched subject not receiving the compound. The precise dosage will vary according to a variety of factors such as subject-dependent variables (e.g., injury size/type, age, joint health, immune system health, etc.), the disease or disorder, and the treatment being administered. The effective amount can be relative to a control. Such controls are known in the art and discussed herein, and can be, for example the condition of the subject prior to or in the absence of administration of the drug.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with HCC are mitigated or eliminated, including, but are not limited to, reducing and/or inhibiting rate of tumor cell proliferation/growth, increasing the quality of life of those suffering from the disease, decreasing the dose of other medications required to treat the disease, delaying the progression of the disease, and/or prolonging survival of individuals.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including neutralizing antibodies against S100A10 may inhibit or reduce the activity and/or quantity of S100A10 by about 10%, 20%, 30%, 40%, 50%, 75%, 85%, 90%, 95%, or 99% from the activity and/or quantity of the same S100A10 in subjects that did not receive or were not treated with the compositions. In some forms, the inhibition and reduction are compared according to the level of mRNAs, proteins, cells, tissues, and organs.

The term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCR, in situ hybridization, Western blotting, and immunostaining such as FACS.

The term “contacting” or “culturing . . . with” is intended to include incubating the component(s) and the cell/tissue together in vitro (e.g., adding the compound to cells in culture) and the step of “contacting” or “culturing . . . with” can be conducted in any suitable manner. For example, the cells may be treated in adherent culture, in suspension culture, or in 3D culture; the components can be added temporally substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art.

### II. Compositions for Targeting S100A10

Compositions for diagnosing and treating liver cancer are provided. Typically, the compositions selectively target S100A10. In some forms, the compositions are suitable for detection of S100A10 in a subject, for example from plasma or serum samples or from tissue biopsy. In other forms, the compositions are suitable for inhibiting and/or reducing the activities and/or quantities of S100A10 in vitro and/or in vivo.

A. S100A10

It has been established that compositions targeting S100A10 identify, treat, and prevent the pathological processes associated with development and progression of liver cancer such as hepatocellular carcinoma.

The S100 group of calcium binding proteins is composed of 21 members that exhibit tissue/cell specific expressions. These S100 proteins bind a diverse range of targets and regulate multiple cellular processes, including proliferation, migration, and differentiation. S100A10, also known as p11, binds mainly to annexin A2 and mediates the conversion of plasminogen to an active protease, plasmin. Higher S100A10 expression has been reported to link to worse outcome and/or chemoresistance in a number of cancer types in lung, breast, ovary, pancreas, gall bladder and colorectum and leukemia although some discrepancy was reported.

In some forms, the S100A10 has suitable for targeting has the following amino acid sequence:

(SEQ ID NO: 1)
MPSQMEHAMETMMFTFHKFAGDKGYLTKEDLRVLMEKEFPGFLE
NQKDPLAVDKIMKDLDQCRDGKVGFQSFFSLIAGLTIACNDYFV
VHMKQKGKK

As shown in the Examples, increased S100A10 has been linked to therapy resistance. Treatment with small molecules that inhibit the S100A10-annexin A2 interaction, antibodies against annexin A2 and S100A10, or the knockdown of S100A10 could all increase the sensitivity of cancer cells to chemotherapy. 1. Antibodies

In some forms, the compositions targeting S100A10 are antibodies that selectively bind S100A10, for example, S100A10 having amino acid sequence of SEQ ID NO:1. In preferred forms, the antibodies are neutralizing antibodies against S100A10. In further preferred forms, the antibodies are effective in reducing or inhibiting proliferation, migration, invasion, motility, and/or metastatic abilities of the cancer cells.

Suitable antibodies may be recombinant, humanized, or non-humanized, polyclonal or monoclonal. These may be antigen binding fragments, single chain, dimeric or multimeric. Methods for producing antibodies are well known in the art. See Antibodies: A Laboratory Manual, Ed Harlow and David Lane eds., (1988), Cold Spring Harbor Laboratory Press, Cold Spring Harbor, New York.

In some forms, the antibodies are mouse anti-human S100A10 antibody available from Santa Cruz (cat no. sc-81153). In preferred forms, the antibodies are humanized mouse anti-human S100A10.

2. Inhibitory Nucleic Acids

In still another form, the compositions contain an inhibitory nucleic acid in an amount effective to downregulate expression of S100A10. Exemplary inhibitory nucleic acids include dsRNA, siRNA, microRNA, or antisense DNA that binds nucleic acids encoding annexin II. While the optimum length of the dsRNA may vary according to the target gene and experimental conditions, the duplex region of the RNA may be at least 19, 20, 21-23, 25, 50, 100, 200, 300, 400, or more bases long.

In some forms, the inhibitory nucleic acid is a small interfering RNAs which lower the expression level of the annexin II gene. A short interfering RNA (siRNA) is a small, double-stranded complex, which triggers the RNAi pathway (Bajan and Hutvagner, Cells, 9(1):137 (2020). In some forms, the siRNA reduces the annexin II expression by about 5%, about 10%, about 25%, about 50%, about 75%, about 80%, about 85%, about 90%, about 95%, about 99%, or about 100%.

In some forms, the inhibitor nucleic acid may be an antisense compound such as an antisense oligonucleotide. Antisense oligonucleotides are single-stranded, highly-modified, synthetic RNA (or DNA) sequences, designed to selectively bind via complementary base-pairing to RNA which encodes the gene of interest (Bajan and Hutvagner, Cells, 9(1):137 (2020).

Exemplary inhibitory nucleic acid sequences include, but are not limited to:

(SEQ ID NO: 2)
CCATGATGTTTACATTTCACACTCGAGTGTGAAATGTAAACATC
ATGG,
(SEQ ID NO: 3)
CCATTGCATGCAATGACTATTCTCGAGAATAGTCATTGCATGCA
ATGG,
(SEQ ID NO: 20)
CCATGATGTTTACATTTCACA,
(SEQ ID NO: 21)
CCATTGCATGCAATGACTATT,
(SEQ ID NO: 22)
GGAGGACCTGAGAGTACTCA,
(SEQ ID NO: 23)
GTAGTACACATGAAGCAGAA,
and
(SEQ ID NO: 24)
CACTCCAAGAACATGACTATT

Some studies have suggested that the knockdown of annexin A2 concurrently results in the loss of S100A10 (Madureira P. A., et al., J. Biomed. Biotechnol. 2012; 2012:353687; and Kwon M., et al., Front. Biosci. 2005; 10:300-325). Thus, in some forms, the compositions contain one or more inhibitory nucleic acids targeting annexin A2. In further forms, the compositions contain one or more inhibitory nucleic acids targeting both S100A2 and annexin A2.

B. Formulations

Formulations of, and pharmaceutical compositions targeting S100A10 including one or more of S100A10 neutralizing antibodies, annexin A2 peptides, small molecule inhibitors, inhibitory nucleic acids, are provided. In some forms, the pharmaceutical compositions can include one or more additional active agents. Therefore, in some forms, the pharmaceutical composition includes two, three, or more active agents. The pharmaceutical compositions can be formulated as a pharmaceutical dosage unit, referred to as a unit dosage form.

Formulations of combination therapies typically include an effective amount of compositions targeting S100A10 including one or more of S100A10 neutralizing antibodies, annexin A2 peptides, small molecule inhibitors, inhibitory nucleic acids.

1. Delivery Vehicles

The active agents can be administered and taken up into the cells of a subject with or without the aid of a delivery vehicle. Appropriate delivery vehicles for the disclosed active agents are known in the art and can be selected to suit the particular active agent. For example, in some forms, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some forms, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some forms, both agents are incorporated into the same particles and are formulated for release at different times and/or over different time periods.

The active agent(s) can be incorporated into a delivery vehicle prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

2. Pharmaceutical Compositions

Pharmaceutical compositions including active agent(s) with or without a delivery vehicle are provided. Pharmaceutical compositions can be for administration by parenteral (intramuscular, intraperitoneal, intravenous (IV) or subcutaneous injection) or enteral routes of administration or using bioerodible inserts and can be formulated in dosage forms appropriate for each route of administration.

In certain forms, the compositions are administered locally, for example, by injection directly into a site to be treated (e.g., into a tumor). In some forms, the compositions are injected or otherwise administered directly into the vasculature onto vascular tissue at or adjacent to the intended site of treatment (e.g., adjacent to a tumor). Typically, local administration causes an increased localized concentration of the compositions which is greater than that which can be achieved by systemic administration. Targeting of the molecules or formulation can be used to achieve more selective delivery.

a. Formulations for Parenteral Administration

Active agent(s) targeting S100A10 such as one or more of S100A10 neutralizing antibodies, annexin A2 peptides, small molecule inhibitors, inhibitory nucleic acids, and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria-retaining filter, by incorporating sterilizing agents into the compositions, by irradiating the compositions, or by heating the compositions.

b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another form, the one or more active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more active agents are formulated into a sold oral dosage form, such as a tablet or capsule, and the solid dosage form is coated with one or more controlled release coatings, such as a delayed release coatings or extended release coatings. The coating or coatings may also contain the active agents.

The extended-release formulations are generally prepared as diffusion or osmotic systems, which are known in the art. A diffusion system typically consists of two types of devices, a reservoir, and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and ® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

Alternatively, extended-release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and capsules containing tablets, beads, or granules. An immediate release portion can be added to the extended-release system by means of either applying an immediate release layer on top of the extended release core using a coating or compression process or in a multiple unit system such as a capsule containing extended and immediate release beads.

Extended-release tablets containing hydrophilic polymers are prepared by techniques commonly known in the art, such as direct compression, wet granulation, or dry granulation processes. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin, and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid, and hydrogenated vegetable oils.

Extended-release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method, and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Preferably, the aqueous solution is water, physiologically acceptable aqueous solutions containing salts and/or buffers, such as phosphate buffered saline (PBS), or any other aqueous solution acceptable for administration to an animal or human Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another form, solvents that are low toxicity organic (i.e., non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one form, compositions may contain minor amounts of polymers, surfactants, or other excipients well known to those of the art. In this context, “minor amounts” means no excipients are present that might affect or mediate uptake of the compounds in the liver and that the excipients that are present in amount that do not adversely affect uptake of compounds in the liver.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

### III. Methods of Diagnosis and Treatment

Compositions and methods for assisting in the diagnosis of liver cancer in a subject are provided. It has been discovered that patients with liver cancer generally have an increased level of S100A10 in the liver tissue and/or in the plasma sample of the patients. Methods of measuring and/or detecting the level and activity of S100A10 in patients are described.

A. Methods of Identifying Subjects with Liver Cancer

The disclosed methods can comprise the steps of a) measuring the level of S100A10 in a sample of a subject; b) comparing the amount of S100A10 in the sample to a control; c) determining whether the sample has an increased level of S100A10 compared to the control to provide an assay output, and d) the subject is identified as having liver cancer if the level of S100A10 is increased compared to the control.

In one form, the methods include the step of obtaining a subject sample. For example, this step could be performed by someone other than the person or machine measuring the levels of the biomarkers. Obtaining the sample can include obtaining the sample directly from the subject or obtaining the sample from a storage area.

In some forms, disclosed are methods that include the step of obtaining the assay output, and prescribing an anti-cancer drug for the subject in a prescription if the amount of the S100A10 is greater than the control. The levels of S100A10 in the disclosed methods can be at least 5%, 10%, 15%, 20%, 25%, 30%, 40%, 50%, 60%, 70%, 80%, 90%, 100% or greater than the control levels. In some forms, the levels of S100A10 can be at least 1.1×, 1.5×, 2×, 2.5×, 3×, 4×, 5×, 6×, 7×, 8×, 9×, 10×, 20×, 30×, 40×, 50×, 60×, 70×, 80×, 90×, 100× or greater than the control levels. In one form, the level of S100A10 can be at least 10% greater than the control levels.

The sample used in the disclosed methods can be a blood sample or plasma sample.

In some forms, the methods include performing an assay. The assay can be done to measure the levels of S100A10. Exemplary assays include an enzyme-linked immunosorbent assay (ELISA), radioimmunoassay (RIA), western blot, and dot blot.

The disclosed methods can include a control which can be a standard. The control can be a sample from a healthy subject or a subject without liver cancer.

In some forms, the methods further comprise the step of transmitting the assay output to a recipient.

Further disclosed are methods where increased levels of S100A10 can provide at least 50%, 60%, 70%, 80%, 90%, 95%, 98%, 99% or 100% confidence or accuracy of the diagnosis or identification of a subject with liver cancer.

Also disclosed are methods of diagnosing liver cancer in a subject including measuring the levels of S100A10 in a sample from the subject and where an increased level of S100A10 in the subject indicating liver cancer in the subject and producing a diagnosis result.

As shown in the Examples, S100A10 is linked with chemotherapy resistance and poor prognosis. Thus, in some forms, the methods stratify HCC patients into a high survival group and a low survival group based on the levels of S100A10 in samples from the subjects. In other forms, the methods stratify cancer patients into responders and non-responders to chemotherapy. In some forms, the patients in the high survival group are responders to conventional chemotherapy. In other forms, the patients in the low survival group are non-responders to conventional chemotherapy.

B. Methods for Treating Liver Cancer

Methods of treating one or more symptoms of liver cancer in a subject are provided. In some forms, the methods include administering to a subject with cancer an effective amount of a therapeutic agent that reduces, inhibits the activity and/or quantify of S100A10. In some forms, the therapeutic agents include one or more of S100A10 neutralizing antibodies, annexin A2 peptides, small molecule inhibitors, inhibitory nucleic acids. In preferred forms, the therapeutic agent is a neutralizing antibody against S100A10.

Data in the Examples showed increased S100A10 levels in cancer cells in the liver and/or in plasma samples of HCC patients. Thus, in some forms, the methods can reduce or inhibit the activity and/or quantify of S100A10 in cancer cells in the liver and/or in the plasma samples. In preferred forms, the methods can reduce or inhibit the activity and/or quantify of S100A10 in the extracellular vesicles (EVs) in the plasma samples of HCC patients.

The methods can effectively decrease or inhibit the proliferation and/or viability of the cancer cells compared to untreated control cancer cells. In some forms, the methods include contacting one or more cancer cells expressing S100A10 with an effective amount of a therapeutic agent, to decrease or inhibit the proliferation and/or viability of the cancer cells compared to untreated control cancer cells. In other forms, the methods include contacting one or more EVs in the plasma and/or liver tissue expressing S100A10 with an effective amount of a therapeutic agent, to decrease or inhibit the proliferation and/or viability of the cancer cells compared to untreated control cancer cells. The therapeutic agent(s) can be administered locally or systemically to the subject, or coated or incorporated onto, or into a device.

In some forms, the cancer may have developed a resistance to the previously administered chemotherapeutic agent(s). Therefore, in some forms, the subject population being treatment is one in which the cancer being treated is resistant or insensitive to one or more conventional chemotherapeutic agents prior to treating with a therapeutic agent that reduces, inhibits the activity and/or quantify of S100A10.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

1. Hepatocellular Carcinoma

In some forms, the methods are effective for treatment of hepatocellular carcinoma (HCC). HCC is the most common primary liver malignancy and is a leading cause of cancer-related death worldwide.

Chronic liver disease and cirrhosis remain the most important risk factors for the development of HCC of which viral hepatitis and excessive alcohol intake are the leading risk factors worldwide.

Chronic viral hepatitis can lead to cirrhosis and/or HCC. Hepatitis B and C are the most common causes of chronic hepatitis in the world. Hepatitis B virus (HBV) is a double-stranded, circular DNA molecule with eight genotypes (A to H). Genotypes A and D are more common in Europe and the Middle East, while genotypes B and C are more common in Asia. Hepatitis B is transmitted via contaminated blood transfusions, intravenous injections, and sexual contact. Vertical transmission from mother to fetus is the leading cause for HBV infection worldwide. Five percent of the world's population is infected with hepatitis B.

Several epidemiological studies have demonstrated significant hepatocarcinogenicity with chronic HBV infection. Hepatitis B carriers have a 10%-25% lifetime risk of developing HCC. Unlike other causes of chronic hepatitis, HBV is unique in that HCC can develop without evidence of cirrhosis. Genotype C has been associated with a higher risk of HCC than genotypes A, B, and D. Active infection with HBV carries an independent risk of HCC with HBV DNA levels >105/mL viral copies associated with a 2.5-3 times increased risk of developing HCC in 8-10 years follow-up. Hepatitis B surface antigen (HBsAg) is not the only hematological marker that carries a significant risk for development of HCC. Patients with positive hepatitis B core antibody (anti-HBc) who are HBsAg-negative also remain at risk for development of HCC. The hepatocarcinogenicity of HBV can be significantly reduced with antiviral treatment for hepatitis B. Suppression of the virus can result in a significant 5-year reduction of the incidence of HCC from 13.7% (controls) to 3.7%, with the greatest reduction occurring in cirrhotic patients.10 The use of HBV vaccination has resulted in significant declines in the incidence of HCC from HBV. The East Asian neonatal vaccination program is estimated to result in a 70%-85% decrease in the incidence of hepatitis B-related HCC. Despite perinatal immunization, 5%-10% of infants remain at risk of acquiring hepatitis B infection. The use of nucleoside analogs in treating chronic hepatitis B mothers in their third trimester of pregnancy has demonstrated superiority to vaccination alone in preventing neonatal transmission.

Hepatitis C virus (HCV) is a small, single-stranded RNA virus, which exhibits high genetic variability. There are six different genotypes of HCV isolated. Genotypes I, II, and III are predominant in the Western countries and the Far East, while type IV is predominant in the Middle East. The highest rates of chronic hepatitis C infection occur in Egypt (18%), with lower rates occur in Europe (0.5%-2.5%), the United States (1.8%), and Canada (0.8%).16 Once infected with HCV, 80% of patients progress to chronic hepatitis, with ˜20% developing cirrhosis. In hepatitis C, the development of HCC occurs almost exclusively in the liver with established cirrhosis; however, in the HALT-C trial, 8% of HCC occurred in patients with only advanced fibrosis. Dual infection with HBV and HCV in a cirrhotic patient increases the risk of HCC with an odds ratio (OR) of 165 compared to 17 for hepatitis C and 23 for hepatitis B alone. A synergistic effect with alcohol increases the incidence of HCC between 1.7- and 2.9-fold when compared to HCV-HCC alone. The risk of HCC is reduced significantly in patients who obtained a sustained viral response after treatment of HCV with a 54% reduction in all-cause mortality. While advances in medications recently have made treating HCV easier, vaccinations against the virus remain elusive.

Alcohol consumption remains an important risk factor for the development of HCC. The relationship between alcohol and liver disease correlates with the amount of alcohol consumed over a lifetime, with heavy alcohol use rather than social drinking being the main risk of HCC. The prevalence rate of alcohol abuse in the United States is five times higher than that of hepatitis C. Alcohol abuse accounts for 40%-50% of all HCC cases in Europe. Studies in Europe reported an increase in the relative risk of developing liver disease above 7-13 drinks per week in women and 14-27 drinks per week in men. In the United States, studies showed that the risk of liver cancer is increased two-to fourfold among persons drinking more than 60 g/d of ethanol. A meta-analysis of 19 prospective studies showed that consumption of three or more drinks per day resulted in a 16% increase risk of liver cancer and consumption of six or more drinks per day resulted in a 22% increase risk.

Sixty percent of patients older than 50 years with diabetes or obesity are thought to have NASH with advanced fibrosis. Chronic medical conditions such as diabetes mellitus and obesity increase the risk of HCC. Diabetes mellitus directly affects the liver because of the essential role the liver plays in glucose metabolism. It can lead to chronic hepatitis, fatty liver, liver failure, and cirrhosis. Diabetes is an independent risk factor for HCC. Patients with diabetes have between a 1.8- and 4-fold increased risks of HCC. It is well-known that obesity is associated with many hepatobiliary diseases, including nonalcoholic fatty liver disease (NAFLD), steatosis, and cryptogenic cirrhosis all of which can lead to the development of HCC.

2. Methods For Selecting Patients

The disclosed methods are suitable for guiding risk classification and treatment strategies in subjects identified as having HCC. Methods can assist in early diagnosis, risk stratification and effective management of patients with HCC. The methods improve the stratification of patients for treatment using therapies that inhibit and/or reduce the activities and/or quantities of S100A10.

In some forms, the therapy includes methods for selecting patients who would be amenable for therapies that inhibit and/or reduce the activities and/or quantities of S100A10, and for treating such patients. In some forms, the methods for selecting patients include the step of selecting a subject having a liver cancer characterized by overexpression of S100A10.

In some forms, the patients who would be amenable for the therapies are those with higher levels of S100A10 in the plasma or tissue samples compared to a healthy control. The levels of S100A10 in the disclosed methods can be at least 5%, 10%, 15%, 20%, 25%, 30%, 40%, 50%, 60%, 70%, 80%, 90%, 100% or greater than the control levels. In some forms, the levels of S100A10 can be at least 1.1×, 1.5×, 2×, 2.5×, 3×, 4×, 5×, 6×, 7×, 8×, 9×, 10×, 20×, 30×, 40×, 50×, 60×, 70×, 80×, 90×, 100× or greater than the control levels. In one form, the level of S100A10 can be at least 10% greater than the control levels.

In other forms, the methods stratify cancer patients into responders and non-responders to therapies that inhibit and/or reduce the activities and/or quantities of S100A10. In some forms, the patients with higher levels of S100A10 in the plasma or tissue samples are likely to be responders to the therapies than those with levels of S100A10 closer to a control.

In some forms, the methods administer to the subject identified as suitable for receiving therapies an effective amount of active agents to inhibit and/or reduce the activities and/or quantities of S100A10 in the subject. In preferred forms, the methods administer to the subject identified as suitable for receiving therapies an effective amount of active agents to reduce or inhibit proliferation, migration, invasion, motility, and/or metastatic abilities of the cancer cells. In further preferred forms, the compositions are effective to reduce or inhibit tumor growth, tumor burden, and/or increase survival of the subject.

3. Dosage and Effective Amounts

Dosage and dosing regimens are dependent on the severity of the disorder and/or methods of administration, and can be determined by those skilled in the art. A therapeutically effective amount of compositions targeting S100A2 or pharmaceutical formulation thereof used in the treatment of liver cancer is typically sufficient to reduce or alleviate one or more symptoms associated with the disease or disorder.

In some forms, the compositions targeting S100A2 are administered in an amount effective to reduce and/or inhibit amount and/or activities of S100A2. In preferred forms, the compositions are effective to reduce or inhibit proliferation, migration, invasion, motility, and/or metastatic abilities of the cancer cells. In further preferred forms, the compositions are effective to reduce or inhibit tumor growth, tumor burden, and/or increase survival of the subject.

In further preferred forms, the disclosed therapeutic agents are administered in an amount effective to reduce or inhibit the number of cancer stem cells (CSCs) in HCC, or the number of cancer cells expressing one or more of CD24, CD44, LGR5, SOX2, c-MYC, CD133, EpCAM, and ABCG2, or the level of expression of one or more of CD24, CD44, LGR5, SOX2, c-MYC, CD133, EpCAM, and ABCG2 on cancer cells.

Epithelial-mesenchymal transition (EMT) is a reversible cellular process, characterized by changes in gene expression and activation of proteins, favoring the trans-differentiation of the epithelial phenotype to a mesenchymal phenotype. This process increases cell migration and invasion of tumor cells, progression of the cell cycle, and resistance to apoptosis and chemotherapy, all of which support tumor progression. Thus, in some forms, the disclosed therapeutic agents are administered in an amount effective to reduce or inhibit EMT. In further forms, the disclosed therapeutic agents are administered in an amount effective to reduce or inhibit activities and/or quantities of one or more kinases associated with EMT including those involved in EGFR, AKT and ERK signaling.

In other forms, the compositions are effective to reduce and/or inhibit annexin A2 phosphorylation. In further forms, the compositions are effective to increase the sensitivity of cancer cells to chemotherapy.

Preferably, the compositions do not target or otherwise modulate the activity or quantity of healthy cells not within or associated with the diseased or target tissues, or do so at a reduced level compared to target cells. In this way, by-products and other side effects associated with the compositions are reduced.

The actual effective amounts can vary according to factors including the specific agent administered, the particular composition formulated, the mode of administration, and the age, weight, condition of the subject being treated, as well as the route of administration and the disease or disorder.

In general, the timing and frequency of administration will be adjusted to balance the efficacy of a given treatment schedule with the side-effects of the given delivery system. Exemplary dosing frequencies include continuous infusion, single and multiple administrations such as daily, weekly, monthly, or yearly dosing.

The composition or pharmaceutical formulation thereof can be administered daily, biweekly, weekly, every two weeks, monthly, or less frequently in an amount to provide a therapeutically effective increase in the blood level of the therapeutic agent. Alternatively, the compositions can be formulated for controlled release, wherein the composition is administered as a single dose that is repeated on a regimen of once a week, or less frequently.

Dosage can vary, and can be administered in one or more doses daily, once daily, twice weekly, once a week, once every two weeks, once monthly, or less frequently. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some forms, the compositions are administered to a subject for between 1 to 20 years, e.g., 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years. Optionally, the compositions are administered for 10 years. In one form, the effects of treatment last for at least 1 year.

In some forms, the regimen includes one or more cycles of a round of therapy followed by a drug holiday (e.g., no drug). The drug holiday can be 1, 2, 3, 4, 5, 6, or 7 days, or 1, 2, 3, 4 weeks, or 1, 2, 3, 4, 5, or 6 months.

In some forms, the amount of compositions administered to a subject changes over time following an initial dose. Therefore, in some forms, the amount of compositions administered to a subject changes over time following an initial dose.

4. Combination Therapy

Early-stage HCC can be treated curatively by local ablation, surgical resection, or liver transplantation. Treatment selection depends on tumor characteristics, the severity of underlying liver dysfunction, age, other medical comorbidities, and available medical resources and local expertise. Catheter-based locoregional treatment is used in patients with intermediate-stage cancer. Kinase and immune checkpoint inhibitors have been shown to be effective treatment options in patients with advanced-stage HCC.

Treatment with one or more active agents that reduce or inhibit the activity and/or quantity of S100A10 can increase the sensitivity of cancer cells to conventional chemotherapy. Thus, in some forms, the therapy is one or more active agents such as small molecules that inhibit the S100A10-annexin A2 interaction, antibodies against annexin A2 and/or S100A10, or inhibitory nucleic acid that knockdown S100A10, in combination with one or more conventional chemotherapeutic agents such as sorafenib, cisplatin, and 5-flurouracil.

The disclosed compositions can be administered to a subject in need thereof, alone or in combination with one or more adjunct therapies or procedures, or can be an adjunct therapy to one or more primary therapies or producers. The additional therapy or procedure can be simultaneous or sequential with the combination therapy. In some forms, the additional therapy is performed between drug cycles or during a drug holiday that is part of the combination therapy dosage regime. In preferred forms, the additional therapy is a conventional treatment for cancer, more preferably a conventional treatment for liver cancer. For example, in some forms, the additional therapy or procedure is surgery, transplant surgery, a radiation therapy, or chemotherapy.

Exemplary additional therapeutic agents include conventional cancer therapeutics such as chemotherapeutic agents, cytokines, chemokines, and radiation therapy. Most chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors). In a particular form, combination therapies used simultaneously or sequentially with a regime of a chemotherapeutic agent, e.g., Gemcitabine (Gemzar), Oxaliplatin (Eloxatin), Cisplatin, Doxorubicin (pegylated liposomal doxorubicin), Capecitabine (Xeloda), Mitoxantrone (Novantrone), docetaxel or cabazitaxel. In some forms, the adjunct or additional therapy is part of the combination therapy.

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sima Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

In some forms, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some forms, the compositions and methods are used prior to or in conjunction with surgical removal of tumors, for example, in preventing primary tumor metastasis. In some forms, the compositions and methods are used to enhance body's own anti-tumor immune functions.

C. Methods for Monitoring and Evaluating Treatment Efficacy

The methods include steps of monitoring the level of S100A10 in a subject. In some forms, the methods include the steps of treating the subject for liver cancer, and then performing any of the disclosed methods to monitor progress of treatment, and/or to detect the level of S100A10 following the treatment.

In some forms, the method further includes discontinuing treatment of the subject if the level of S100A10 is reduced, for example by 10%, 20%, 30%, 40%, 50%, 60%, 70%, 80%, 90%, or more than 90% compared to the level prior to treatment.

D. Methods for Drug Screening

There is a lack of effective treatments available for liver cancer. The systems and methods are useful to investigate the activity or applicability of one or more test compounds to treat or alleviate or prevent one or more symptoms of liver cancer. Therefore, in some forms, the methods include one or more steps for assessing the quantity and/or activities of S100A10 in the presence of one or more active agents, where the quantity and/or activities of S100A10 in the presence of the active agent is assessed by comparison with the quantity and/or activities of S100A10 in the absence of the active agent. In an exemplary form, an active agent is selected if it is effective to reduce the quantity and/or activities of S100A10.

### IV. Kits

Kits are also disclosed. The kits can include some or all of the materials needed to measure the levels of S100A10. The kit can contain a positive and a negative control. The kits can include printed instructions for use.

The present invention is further understood by reference to the following non-limiting paragraphs:
1. A method for identifying a subject as having an elevated risk of having hepatocellular carcinoma (HCC), the method comprising:
determining the level of S100A10 in the sample obtained from the subject,
identifying the subject as having HCC if the level of S100A10 in the sample is increased compared to the level of S100A10 in a control sample.
2. The method of paragraph 1, wherein the sample is a plasma sample or a liver biopsy.
3. The method of paragraph 1 or 2, wherein the control sample is a sample from a healthy subject.
4. The method of any one of paragraphs 1-3, wherein the level of S100A10 is at least 10% greater than the control.
5. The method of any one of paragraphs 1-4, wherein the step of determining the level of S100A10 in the sample comprises measuring the level of S100A10 protein in the sample.
6. The method of any one of paragraphs 1-5, wherein the step of determining the level of S100A10 in the sample comprises measuring the level of S100A10 protein derived from the extracellular vesicles of the sample.
7. The method of any one of paragraphs 1-6, wherein the step of determining the level of S100A10 in the sample comprises performing an enzyme-linked immunosorbent assay (ELISA), radioimmunoassay (RIA), western blot, and dot blot.
8. The method of any one of paragraphs 1-4, wherein the step of determining the level of S100A10 in the sample comprises measuring the level of S100A10 mRNA in the sample.
9. The method of paragraph 8 further comprising performing a hybridization assay, Real-time Polymerase chain reaction (RT-PCR), or Quantitative Polymerase chain reaction (qPCR).
10. The method of any one of paragraphs 1-9, wherein the step of identifying the subject as having HCC is such identification with at least a 50%, 60%, 70%, 80%, 90%, 95%, 97%, or 99% certainty.
11. The method of any one of paragraphs 1-10 further comprising the step of treating the identified subject by administering a therapeutic agent for treating HCC.
12. The method of paragraph 11, wherein the therapeutic agent is one or more selected from the group consisting of antibodies, small molecule inhibitors, and inhibitory nucleic acids.
13. The method of paragraph 12, wherein the small molecules are effective in inhibiting the S100A10-annexin A2 interaction.
14. The method of paragraph 12, wherein the antibodies are neutralizing antibodies against annexin A2 and/or S100A10.
15. The method of paragraph 12, wherein the inhibitory nucleic acids are shRNA targeting S100A10.
16. A method of treating a subject having hepatocellular carcinoma (HCC), the method comprising:
determining the level of S100A10 in a sample obtained from the subject,
administering to the subject an effective amount of a therapeutic agent for treating HCC if the level of S100A10 in the sample is increased compared to the level of S100A10 in a control sample.
17. The method of paragraph 16, wherein the level of S100A10 is at least 10%, 20%, 30%, 40%, 50%, 60%, 70%, 80%, 90%, 100%, or more than 100% greater than the control.
18. The method of paragraph 16 or 17, wherein the therapeutic agent is one or more selected from the group consisting of antibodies, small molecule inhibitors, and inhibitory nucleic acids.
19. The method of paragraph 18, wherein the small molecules are effective in inhibiting the S100A10-annexin A2 interaction.
20. The method of paragraph 18, wherein the antibodies are neutralizing antibodies against annexin A2 and/or S100A10.
21. The method of paragraph 18, wherein the inhibitory nucleic acids are shRNA targeting S100A10.
22. The method of any one of paragraphs 16-21, wherein the therapeutic agent is administered in an amount effective to reduce or inhibit activities and/or quantities of S100A10.
23. The method of any one of paragraphs 16-22, wherein the therapeutic agent is administered in an amount effective to reduce or inhibit the quantity of extracellular vesicles expressing S100A10 in the blood of the subject.
24. The method of any one of paragraphs 16-23, wherein the therapeutic agent is administered in an amount effective to reduce or inhibit proliferation, migration, invasion, motility, and/or metastatic abilities of the cancer cells of HCC.
25. The method of any one of paragraphs 16-24, wherein the therapeutic agent is administered in an amount effective to reduce or inhibit tumor growth, tumor burden, and/or increase survival of the subject.
26. The method of any one of paragraphs 16-25, wherein the therapeutic agent is administered in an amount effective to reduce or inhibit the number of cancer stem cells (CSCs) in HCC, or the number of the cancer cells expressing one or more of CD24, CD44, LGR5, SOX2, c-MYC, CD133, EpCAM, and ABCG2, or the level of expression of one or more of CD24, CD44, LGR5, SOX2, c-MYC, CD133, EpCAM, and ABCG2 on the cancer cells.
27. The method of any one of paragraphs 16-26, wherein the therapeutic agent is administered in an amount effective to reduce or inhibit activities and/or quantities of one or more kinases of EGFR, AKT and ERK signaling associated with epithelial-mesenchymal transition of the cancer cells.

### EXAMPLES

### Example 1

Materials and Methods

Human Samples

Plasma samples were randomly collected from HCC patients without treatment and healthy subjects without liver disease background. Human HCCs and their paired non-tumorous liver (NT) tissues were collected during surgical resection at Queen Mary Hospital, Hong Kong, with informed consent from patients. Procedure approval was obtained from the Institutional Review Board of The University of Hong Kong. All experiments involving human samples were handled in accordance with relevant ethical regulations.

Cell Lines and Culture Conditions

HCC cell line PLC/PRF/5 (CRL-8024) was obtained from American Type Culture Collection (ATCC). HCC cell line Huh7 (JCRB0403) was obtained from JCRB Cell Bank. MHCC97L and MHCC97H were gifts from Liver Cancer Institute, Fudan University. MIHA was kindly provided by Dr. J. R. Chowdhury, Albert Einstein College of Medicine, New York. MHCC97L cells were cultured in Dulbecco's modified Eagle minimal high glucose essential medium (DMEM-HG) supplemented with 1 mM NaPy. PLC/PRF/5, Huh7, MIHA and HEK293FT cells were cultured in DMEM-HG media. All cell culture media mentioned above were further supplemented with 10% fetal bovine serum (FBS), 1% penicillin and 1% streptomycin unless otherwise specified. Cell line cultures were maintained in 37oC and 5% CO2 incubator.

Authentication of HCC cell lines used in this study was performed by short tandem repeat (STR) DNA Profiling in March 2018 and no cellular cross-contamination was detected. STR results for MHCC97L is provided in Table 4. Cell cultures were tested negative for Mycoplasma contamination. “Xenome”, utilizing our RNA-seq data, estimated a negligible 0.04% to 0.42% (n=3) “mouse-likely” reads (probably an artifact instead of real contamination) for MHCC97L, which was comparable to the 0.15% to 0.40% for clinical human NTL and HCC samples (n=6), thus indicating our MHCC97L cells do not contain cells of murine origin (1). Furthermore, MHCC97L used in this study contains HBV integrated in TERT locus of the genome (2).

Lentiviral-Based S100A10 Overexpression Cells and CRISPR/Cas9 Mediated S100A10-KO Cells

S100A10 lentiviral-based expression constructs using pCDH-EF1-MCS-IRES-Puro vectors (System Biosciences) were prepared by standard molecular cloning techniques. Lenti-Guide-Puro based lentiviral single guide RNA (sgRNA) expression vector (Provided by Dr. Zheng Feng, MIT) carrying specific S100A10 targeting sequence (sgS100A10) or GFP targeting control sequence (sgGFP) transfected into 293FT packaging cells to produce viral containing supernatant for subsequent HCC cells viral transduction of Cas9 expressing MHCC-97H cells transduced by lenti-Cas9-Blast system (Dr. Zhang Feng, MIT). To establish S100A10 KD, two short hairpin RNAs (shRNA) specifically targeting S100A10 (shS100A10-1, and shS100A10-3) were cloned into the PLL3.7 lentiviral vector (Addgene). Stably transduced MHCC97L cells were selected by puromycin (Sigma-Aldrich). Shorten tandem repeat (STR) DNA profiling of MHCC97L authenticates there is no contamination of the cells as described in Table 4.

TABLE 4
Shorten tandem repeat (STR) DNA profiling of MHCC97L,
authenticating no contamination.

| | | MHCC97L |
|---|---|---|
| DNA Marker | MHCC97L⁴ | (L-171218744P) |
| AMEL | X, Y | X, Y |
| CSF1PO | 11, 13 | 11, 13 |
| D13S317 | 8 | 8 |
| D16S539 | 12 | 12 |
| D5S818 | 12, 13 | 12, 13 |
| TH01 | 10 | 10 |
| TPOX | 8 | 8 |
| vWA | 14 | 14 |
| D18SS51 | — | 13, 22 |
| D21S11 | — | 31, 2 |
| D3S1358 | — | 15, 16 |
| D8S1179 | — | 12, 13 |
| FGA | — | 21, 24 |
| Penta D | — | 8, 9 |
| Penta E | — | 11, 17 |

| | |
|---|---|
| Number of shared alleles | 12 |
| Total number of alleles in the reference profile | 12 |
| Percet match | 100% |

Focus Formation Assay

Focus formation assay was used to assess anchorage-dependent growth. In brief, 1,000 cells were seeded in each well of six-well plate for approximately 2-3 weeks. Surviving colonies were stained and counted using crystal violet (Sigma-Aldrich).

Sphere Formation Assay

A total of 1,000 cells were cultured in 0.25% methyl cellulose (Sigma-Aldrich) supplemented DMEM/F12 medium (Life Technologies) with 20 ng/mL EGF (Life Technologies), 10 ng/mL basic FGF (Life Technologies), B27 (1:50, GIBCO), and 4 μg/mL insulin (BIOIND, Kibbutz Beit Haemek, Israel) in 24-well plates, which were coated with poly HEMA (Sigma-Aldrich). The cells were replenished with 30 μL supplementary medium every other day.

Cell Motility Assays

Transwell migration and invasion assays were performed to evaluate cell motility (Corning, #353097 for migration, #354480 for invasion). Approximately 1×105 cells were seeded in the medium without FBS on transwell upper chambers, and the lower chamber was supplied with medium with 10% FBS, with the indicated conditional medium, or EVs. The cells that migrated and invaded to the lower membrane surface were stained using crystal violet and then counted under microscopy.

Animal Studies

All animal experiments were conducted and approved by the University of Hong Kong Committee on the Use of Live Animals in Teaching and Research (CULATR). The frequency of CSCs with tumor initiation capabilities was evaluated using limiting dilution assay. The BALB/c nude mouse xenograft model was used to evaluate tumorigenicity and chemoresistant ability in vivo. Nude mice were intrasplenically injected with HCC cells to assess the liver metastatic capacity in vivo. After surgery, mice recovered from anesthesia in a cage under a heater. Analgesia was provided to the nude mice during the first postoperative week. Tail vein injection of luciferase-labelled MHCC-97L cells in NOD SCID mice was used to assess lung metastatic ability. After mice were killed, the tumor tissues, livers, spleens, and lungs were excised and fixed in 4% paraformaldehyde overnight. Thereafter, fixed tissues were embedded in paraffin for further studies.

For chemoresistance in xenograft model, subcutaneous xenografts in nude mice were established with PLC/PRF/5 or MHCC97L cells. Treatment was started once the size of the xenograft reached ˜5 mm in diameter. The mice were randomly assigned into different groups, each consisting of at least 5 mice. Sorafenib at 10 mg/kg was administered daily through oral gavage. Mouse IgG antibody or anti-S100A10 antibody was administered at 10 μg once every 3 days by intraperitoneal injection.

Chemotherapy-Induced Cytotoxicity and Apoptotic Assay

Sorafenib, cisplatin or 5-FU induced cytotoxicity was determined by XTT Cell Proliferation Assay (Roche Diagnostics) according to the manufacturer's instructions. The apoptotic assay was determined by flow cytometry using annexin-V staining. After treating with Sorafenib, cisplatin or 5-FU for 48 h, the cells were collected and double stained with FITC-conjugated Annexin-V and PI provided in the BD apoptosis detection kit (BD Biosciences).

Isolation of EVs from Cell Culture Medium and Plasma of HCC Patients

For EVs isolation from cell culture supernatants, HCC cells were cultured in medium with 10% EV-depleted FBS, which was prepared by 100,000× g centrifugation overnight (≥12 h) at 4° C. (Himac, CP100NX Ultracentrifuges). EVs were purified by differential centrifugation after the cell culture supernatant were collected. Briefly, cell culture supernatants were centrifuged at 2000×g for 15 min to remove cell debris and dead cells. Then the supernatant was centrifugated at 20000×g for 30 min at 4° C. to remove microvesicles. Then the supernatant was first passed through 0.22 μm filter followed by ultracentrifugation at 100,000×g for 2 h at 4° C. to collect EVs. The EVs were then washed with PBS and collected by ultracentrifugation at 100,000×g for another 2 h at 4° C. HCC patients' or healthy donors' plasma derived EVs were purified by differential centrifugation. The plasma was first centrifuged at 4000 g for 15 min to obtain cell-free plasma. Then, 500 μL of the obtained plasma was toped-up to 1 mL and then centrifuged at 20,000 g for 1 hour (Himac, CP100NX Ultracentrifuges). The collected supernatants were then centrifuged at 100,000 g for 2 h at 4° C. (Himac, CP100NX Ultracentrifuges) to pellet the EVs. The EVs were then washed with PBS and collected by ultracentrifugation at 100,000×g for another 2 h at 4° C.

EV Characterization

The morphology and integrity of EVs is observed by electronic microscope. In brief, EVs suspended in PBS were dropped on formvar carbon-coated nickel grids and stained with 2% uranyl acetate. The EVs were then visualized by Philips CM100 transmission electron microscope (FEI Company). Target proteins present on EVs were determined by immunogold staining followed by visualizing by transmission electron microscope. Protein of isolated EVs was examined by western blotting by EV specific markers CD63 (Abcam, 134045), CD81 (Abcam, #79559), CD9 (Abcam, #92726), HSP70 (Abcam, #181606), Alix (Santa Cruz, #53540), TSG101 (BD Biosciences, #612696) and EVs negative markers GM130 (Abcam, #52649) and p62 (Abcam, 140651). The size distribution of EVs and particle concentration was measured by ZetaView BASIC NTA PMX-120 (Particles Metrix GmbH).

EV Education Mouse Model

To investigate the role EVs in tumor liver metastasis, 6-week-old BALB/c nude male mice were intrasplenic injected with HCC cells. After the implantation of tumor cells, 10 μg EVs or PBS was intravenously injected every 4 days for 1 month. Autopsies were performed after 8-10 weeks and the presence of metastases was examined macroscopically. For lung metastasis, NOD SCID mice were injected intravenously with luciferase-labelled HCC cells and 10 μg EVs or PBS as control. Then the EVs or PBS was educated to mice every 4 days for 1 month. Lung metastasis was monitored using bioluminescence imaging at around 8-10 weeks.

Labeling of EVs for Uptake Analysis

EVs were fluorescently labeled with PKH26 Membrane Dye Labeling Kit (Sigma Aldrich, #PKH26GL) according to manufacturer's protocol. Labeled EVs were washed with PBS and collected by ultracentrifugation as described above. To assess tissue distribution of EVs, NOD SCID mice were injected intravenously with 15 μg of PKH26-labeled EVs. Each mouse was anesthetized and perfused to collect lung, liver, spleen, heart and kidney. Tissue sections from different organs were stained with DAPI and examined under LSM900 confocal microscopy (Carl Zeiss). Three random fields of each section were captured and three sections per organ were examined

Pulmonary Leakiness Assay

Male 6-week-old NOD SCID mice were injected intravenously with 15 μg of EVs or PBS as control. They were intravenously injected with Texas Red lysine-fixable dextran (Invitrogen, #D1864, 70,000MW) at 100 mg/kg post 24 hr EV injection. Mice were intravenously injected with Alexa Fluor 488 concanavalin A (Invitrogen, #C11252) at 10 mg/kg after 3 hr. They were anesthetized 10 min later and lung tissues were excised. Tissues were cryosectioned at 12 μm thickness. Tissue sections were stained with DAPI (Invitrogen, #D1306) and examined under LSM900 confocal microscopy (Carl Zeiss) for vascular leakage. Three random fields of each section were captured and 3 sections per lung were examined

Treatment of HCC Cells Before Functional Assays

HCC cells were seeded in 6-well plate and subjected to 2 μg EVs treatment for 48 hours one day after seeding. Then the cells were used for functional studies or western blotting. For treating with neutralizing antibodies, EVs were incubated with PBS (1:10 volume/volume), IgG antibody (1:10 volume/volume) (Santa Cruz, sc-2025), or anti-S100A10 neutralizing antibody (1:10 volume/volume) (Santa Cruz, sc-81153) before they were applied in in vitro or in vivo assays. For S100A10 NA or ITGAV treatment before EVs isolation, 20 ug S100A10 NA or IgG were pre-incubate with 1,000 million cells were pre-incubate with 1,000 million cells and then seed in medium with 10% EVs free FBS. Then the EVs were collected after 48 hours.

Quantitative Real-Time PCR (qRT-PCR)

Total RNA was extracted using the TRIZOL Reagent (Life Technologies). cDNA was synthesized by reverse transcription (Roche). SYRB Green PCR Master Mix (Applied Biosystems) and Applied Biosystems Quant Studio 5 Real-time PCR System were used for qRT-PCR analysis. All qRT-PCR reactions were tested in triplicates. Primers used in this study are listed in Table 5.

TABLE 5
List of Primers used in the Study

| | | |
|---|---|---|
| Gene | Primer | Sequence |

For Real-time PCR

| | | |
|---|---|---|
| S100A10 | Forward | TCGCTGGGGATAAAGGCTAC |
| | | (SEQ ID NO: 4) |
| | Reverse | AAGAAGCTCTGGAAGCCCAC |
| | | (SEQ ID NO: 5) |
| CD24 | Forward | GCTCCTACCCACGCAGATTT |
| | | (SEQ ID NO: 6) |
| | Reverse | GAGACCACGAAGAGACTGGC |
| | | (SEQ ID NO: 7) |
| CD44 | Forward | TGCCGCTTTGCAGGTGTAT |
| | | (SEQ ID NO: 8) |
| | Reverse | GGCCTCCGTCCGAGAGA |
| | | (SEQ ID NO: 9) |
| LGR5 | Forward | CCCGAATCCCCTGCCCAGTCT |
| | | (SEQ ID NO: 10) |
| | Reverse | TCATCCAGCCACAGGTGCCTA |
| | | (SEQ ID NO: 11) |
| SOX2 | Forward | AAATGGGAGGGGTGCAAAAGAGGAG |
| | | (SEQ ID NO: 12) |
| | Reverse | CAGCTGTCATTTGCTGTGGGTGATG |
| | | (SEQ ID NO: 13) |
| C-MYC | Forward | CGTCCTCGGATTCTCTGCTC |
| | | (SEQ ID NO: 14) |
| | Reverse | GCTGGTGCATTTTCGGTTGT |
| | | (SEQ ID NO: 15) |
| ABCG2 | Forward | TCATCAGCCTCGATATTCCATCT |
| | | (SEQ ID NO: 16) |
| | Reverse | GGCCCGTGGAACATAAGTCTT |
| | | (SEQ ID NO: 17) |
| GAPDH | Forward | GGAGCGAGATCCCTCCAAAAT |
| | | (SEQ ID NO: 18) |
| | Reverse | GGCTGTTGTCATACTTCTCATGG |
| | | (SEQ ID NO: 19) |

Gene Copy Number Variation Assay

TaqMan probe-based gene copy number assay was used to quantify the copy number variation (CNV) of S100A10. Copy number of S100A10 targeting intron 1 (Hs06508510_cn, Cat. No. 4400292, Life Technologies, CA, USA) was measured. TaqMan™ Copy Number Reference Assay, human, RNase P (Life Technologies, #4403326) was used as internal reference control. The CNV of paired HCC and their corresponding non-tumorous liver (NT) tissues were normalized to normal liver tissue.

Western Blotting Analysis

Quantified protein lysates were resolved on SDS-PAGE, transferred onto a polyvinylidenedifluoride (PVDF) membrane (Bio-Rad), and then blocked with 5% non-fat milk in Tris-buffered saline-Tween 20 (TBST) for 1 h at room temperature. The blocked membrane was then incubated with primary antibody diluted in 5% bovine serum albumin in TBST at 4° C. overnight. Antibodies used in this study are listed in Table 6.

TABLE 6
List of antibodies used in this study.

| Antibody | Application | Sourceª | Cat No. |
|---|---|---|---|
| Anti-human S100A10 | WB, IP, | Sigma | HPA003340 |
| | Immunogold | | |
| Anti-human CD63 | WB | Abcam | ab134045 |
| Anti-human CD63 | Immunogold | Abcam | ab271286 |
| Anti-human CD9 | WB | Abcam | ab92726 |
| Anti-human CD81 | WB | Abcam | ab79559 |
| Anti-human TSG101 | WB | BD | 612696 |
| Anti-human Alix | WB | Santa Cruz | sc-53540 |
| Anti-human GM130 | WB | Abcam | ab52649 |
| Anti-human p62 | WB | Abcam | ab140651 |
| Anti-human HSP70 | WB | Abcam | ab181606 |
| Anti-human S100A10 | Neutralization | Santa Cruz | sc-81153 |
| Normal mouse IgG | Neutralization | Santa Cruz | sc-2025 |
| Anti-human EGFR | Neutralization, | Sigma | 05-101 |
| | WB | | |
| Anti-human EGFR | WB | Abcam | ab40815 |
| (phospho Y1068) | | | |
| Anti-human EGF | WB, | Santa Cruz | sc-374255 |
| | immunogold | | |
| Anti-human ITGAV | WB, | Abcam | ab179475 |
| | immunogold | | |
| Anti-human MMP2 | WB, | Abcam | ab97779 |
| | immunogold | | |
| Anti-human E-cadherin | WB | Cell signaling | 3195 |
| Anti-human Fibronectin | WB, | Abcam | ab2413 |
| | immunogold | | |
| Anti-human N-cadherin | WB | Cell signaling | 13116 |
| Anti-human Vimentin | WB | Cell signaling | 5741 |
| Anti-human p44/42 | WB | Cell Signaling | 4695 |
| MAPK (ERK1/2) | | | |
| Anti-human Phosphop44/ | WB | Cell signaling | 4370 |
| 42 MAPK (ERK1/2) | | | |
| Anti-human Akt | WB | Cell signaling | 9272 |
| Anti-human phospho-Akt | WB | Cell signaling | 9271 |
| (Ser473) | | | |

TABLE 7
List of target sequences for knockdown and
knockout of genes.

| sh/sgRNA | Gene | |
|---|---|---|
| clones | name | Target sequence |
| shS100A10#1 | S100A10 | CCATGATGTTTACATTTCACA |
| | | (SEQ ID NO: 20) |
| shS100A10#3 | S100A10 | CCATTGCATGCAATGACTATT |
| | | (SEQ ID NO: 21) |
| sgS100A10#2 | S100A10 | GGAGGACCTGAGAGTACTCA |
| | | (SEQ ID NO: 22) |
| sgS100A10#4 | S100A10 | GTAGTACACATGAAGCAGAA |
| | | (SEQ ID NO: 23) |
| shITGAV | ITGAV | CACTCCAAGAACATGACTATT |
| | | (SEQ ID NO: 24) |

Gelatin Zymography

50 μg un-denatured EVs or 20 ng of activated recombinant MMP-2 (Abeam, #81550) was loaded with Laemmli Sample Buffer (Bio-Rad, #1610747) onto 10% gelatin zymogram protein gels (Thermo, #ZY00102BOX) and run at 100V for 1.5 h in Tris-Glycine SDS Running Buffer (Thermo, #LC2675). Gels were then incubated with 1× renaturing buffer (Thermo, #LC2670) for 30 min at room temperature and then incubated with 1× developing buffer (Thermo, #LC2671) overnight at 37° C. Developed gels were then gently washed with H₂O and stained with Coomassie Blue dye.

Statistical Analysis

The SPSS version 17.0 (SPSS, Inc., Chicago, IL) was used for data analysis. The mRNA level of S100A10 in paired tumor and adjacent nontumor tissues was compared with a paired Student t test. Patients' survival rates were analyzed using Kaplan-Meier plots and log-rank tests. The correlations between different clinicopathological parameters were evaluated using Pearson's χ2 test. The frequency of CSCs with tumor initiation capabilities were calculated by limiting dilution assay in the ELDA software(Hu and Smyth, 2009). Data are presented as the mean±SD of three independent experiments. Results were considered statistically significant for P values <0.05.

Results

Clinical Significance of S100A10 in HCC

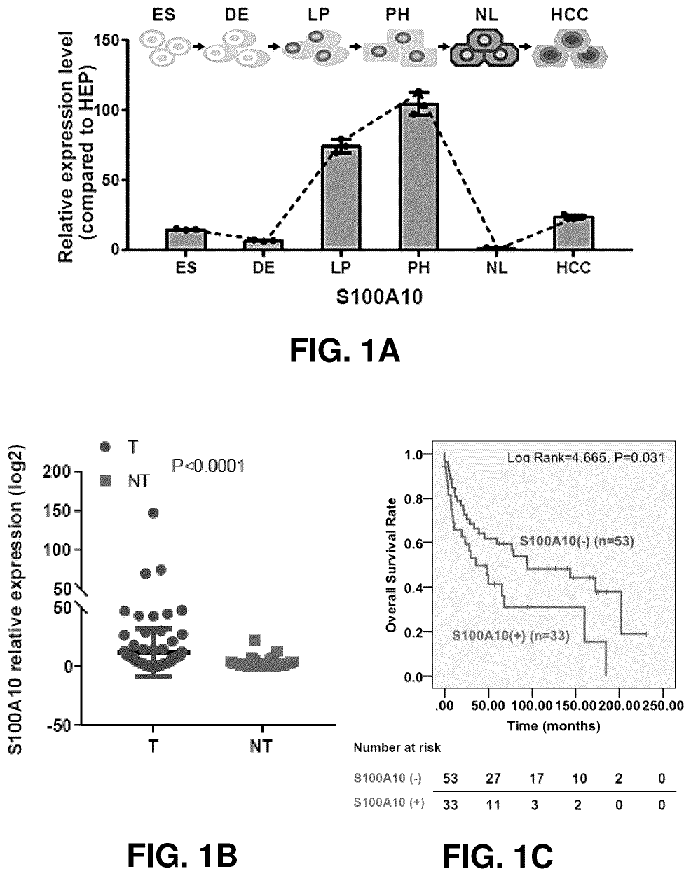

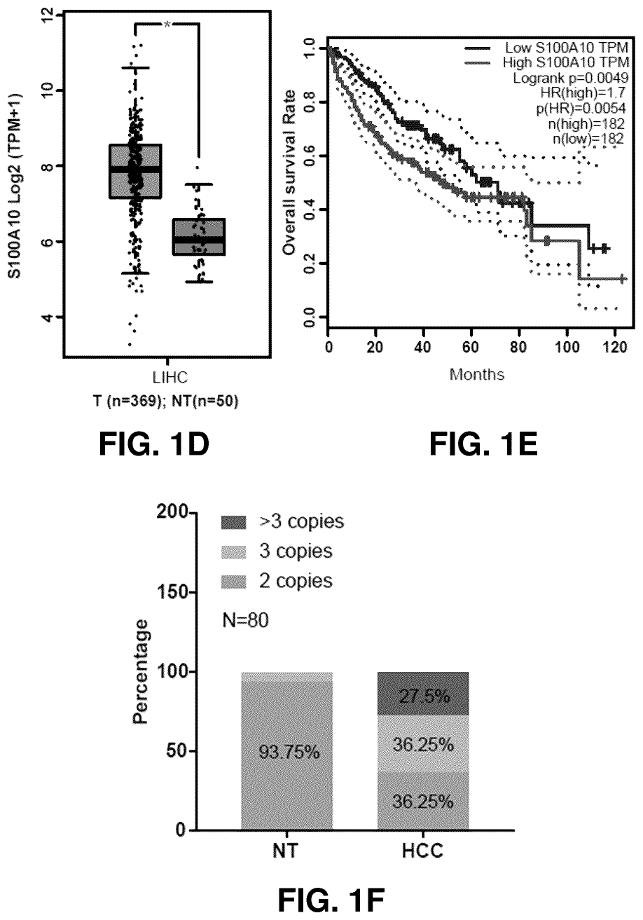

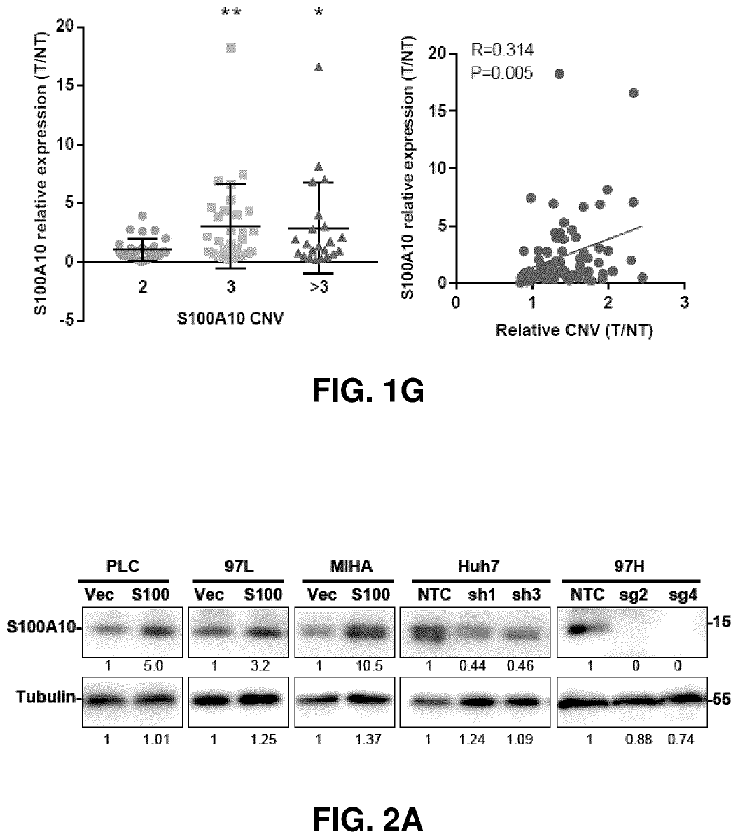

In this study, when analysis was done on the in vitro liver differentiation model derived from human embryonic stem cells reported previously,′ 2 it was found that S100A10 was highly expressed in the liver progenitor and premature hepatocyte stages when compared with mature hepatocytes (FIG. 1A). The expression of S100A10 was then validated by real-time reverse transcription-PCR (qRT-PCR) in a cohort of 86 paired HCC clinical samples. S100A10 was highly expressed in HCC tumours as compared with the non-tumorous livers (FIG. 1B), with an upregulation by ≥2 folds in 38.4% (33/86) of tumours. In addition, high expression of S100A10 was associated with poorer prognosis with significantly shorter overall survival rates (FIG. 1C); similar results were observed in The Cancer Genome Atlas (TCGA) database (FIGS. 1D and 1E). On clinicopathologic correlation, higher expression of S100A10 was significantly associated with more aggressive tumour behaviour including more frequent venous invasion (p=0.044) and poorer cellular differentiation (p=0.047) (Table 1). From TCGA database, high expression of S100A10 in HCCs is significantly associated with hepatitis B/C infection (Table 2). As S100A10 is located on chromosome 1q21, which is frequently amplified in multiple types of cancer, 3-5 21 the copy number variation (CNV) of S100A10 from the genomic DNA of 80 HCC samples from this cohort was analyzed. Most (63.75%, 51/80) of the tumours showed CNV of ≥3 copies, with 27.5% showing 4 or more copies (FIG. 1F and FIG. 9). Furthermore, the S100A10 expression levels in patients' tumours with copy number amplification were significantly higher than those without, and the relative CNV and S100A10 expression were significantly and positively associated (FIG. 1G).

TABLE 1
Association of S100A10 Expression with
Clinicopathologic features in 86 HCC Cases

| | | S100A10 expression* | | |
|---|---|---|---|---|
| Features | Total | Low | High | P-value |
| Sex | | | | 0.798 |
| Male | 66 (76.7%) | 40 (75.5%) | 26 (78.8%0 | |
| Female | 20 (23.3%) | 13 (24.5%) | 7 (21.2%) | |
| Age, years | | | | >0.999 |
| ≤60 | 54 (62.8) | 33(62.3%) | 21 (63.6) | |
| >60 | 32 (37.2) | 20 (37.6%) | 12 (36.4) | |
| Venous Invasion | | | | 0.047 |
| Absent | 49 (57.0%) | 35 (66.0%) | 14 (42.4%) | |
| Present | 37 (43.0%) | 18 (34.0%) | 19 (57.6%) | |
| Differentiation | | | | 0.044 |
| Well/Moderate | 38 (44.2%) | 28 (52.8%) | 10 (30.3%) | |
| Poor | 48 (55.8%) | 25 (47.2%) | 23 (%) | |
| TNM Stage(AJCC) | | | | .241 |
| I/II | 29 (33.7%) | 15 (28.3%) | 14 (42.4%) | |
| III-VI | 57 (66.3%) | 38 (71.7%) | 19 (57.6%) | |
| Serum HBsAG | | | | 0.703 |
| Negative | 7 (8.1%) | 5 (9.4%) | 2 (6.1%) | |
| Positive | 79(91.9%) | 48 (90.6%) | 31 (93.9%) | |
| Cirrhosis | | | | 0.145 |
| Absent | 2 (2.3%) | 0 (0%) | 2 (6.1%) | |
| Present | 84 (97.7%) | 53 (100%) | 31 (93.9%) | |

NOTE:
Statistical significance (P < 0.05) is shown in bold.
S100A10low: <2 folds; high ≥2 folds.
Fischer exact test.

TABLE 2
Association of S100A10 expression with
clinicopathologic features in TCGA.

| | | S100A10 expression | | |
|---|---|---|---|---|
| Features | Total | Low | High | P-value |
| Tumor Stage (AJCC) | 347 | | | 0.714 |
| Stage 1-11 | 257 (74.1%) | 125 (73.1%) | 132 | |
| II-IV | 90 (25.9%) | 46 (26.9%) | 44 | |
| Virus Infection | 352 | | | <0.001 |
| Absent | 199 (56.5%) | 117 (66.5%) | 82 (46.6%) | |
| Present | 153 (43.5%) | 59 (33.5%) | 94 (53.4%) | |
| Alcohol Uptake | 352 | | | 0.258 |
| Yes | 117 (33.2%) | 53 (30.1%) | 64 | |
| No | 235 (66.8%) | 123 (69.9%) | 112 | |

S100A10 Enhances the Sternness Characteristics of HCC

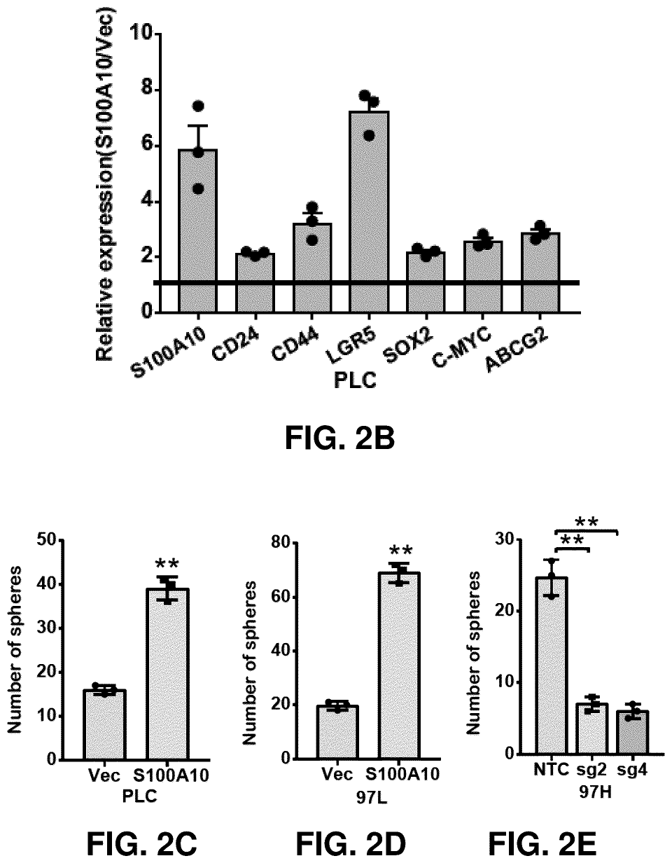

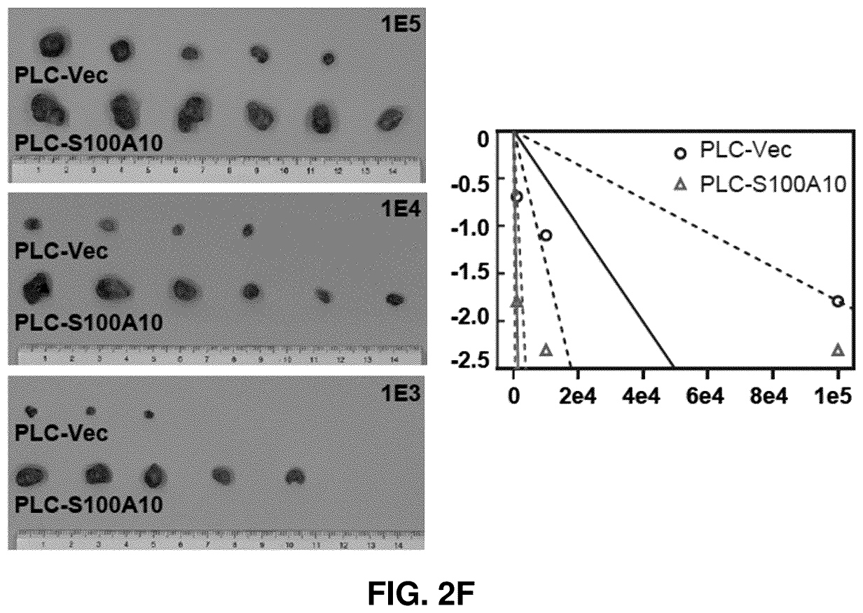

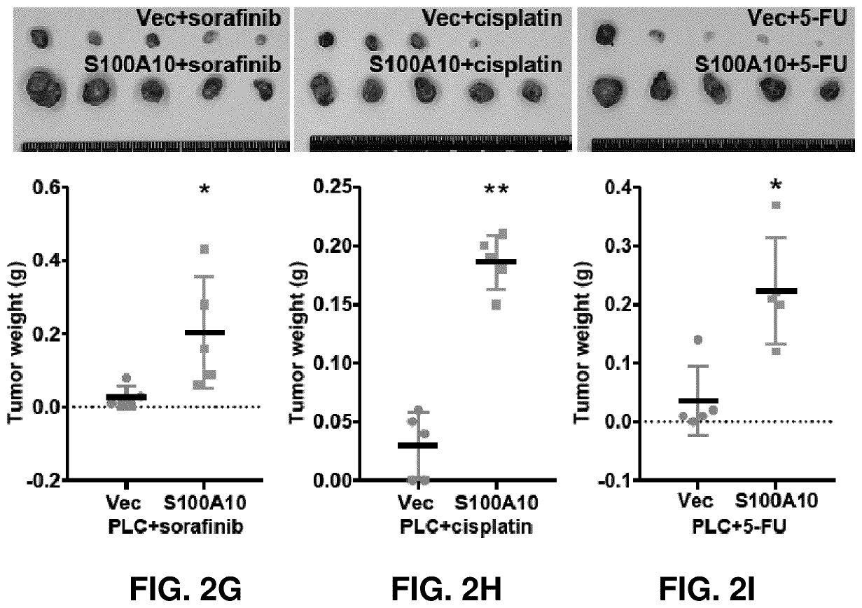

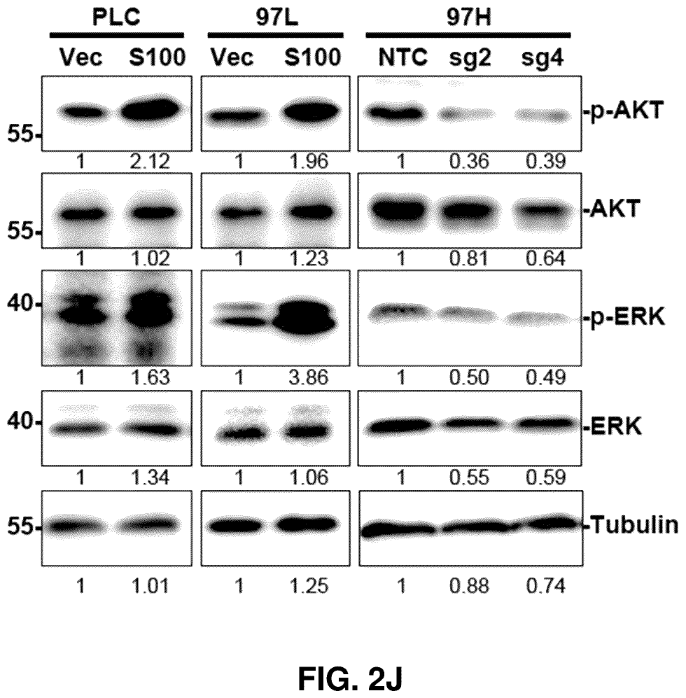

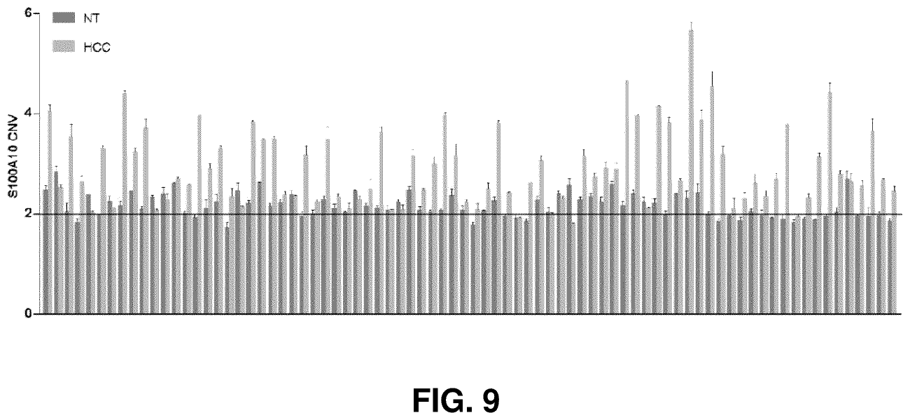

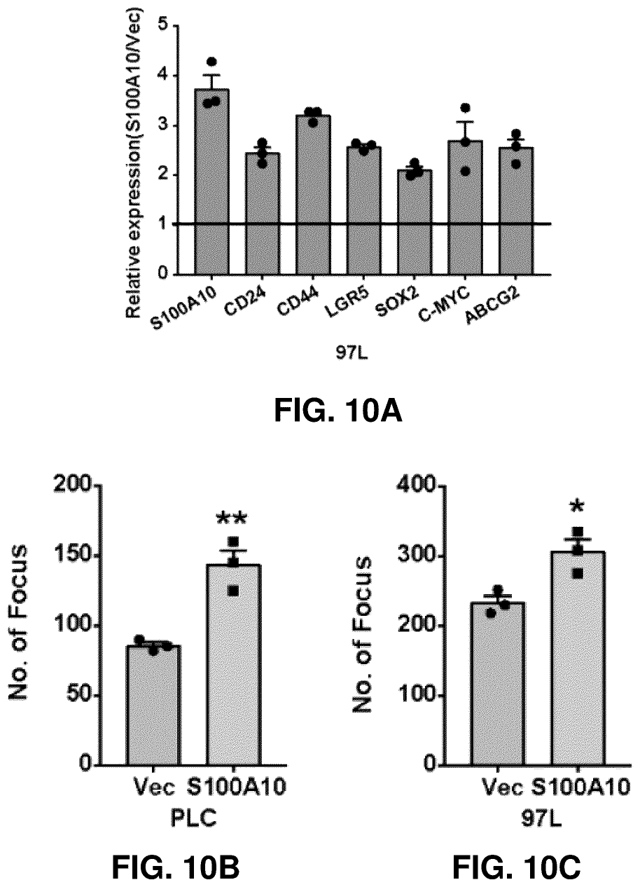

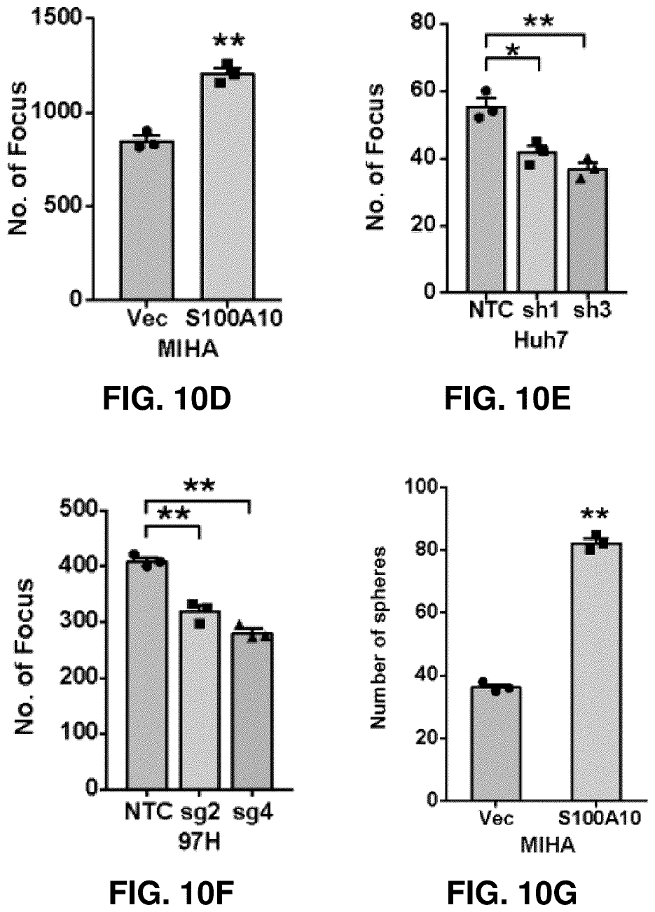

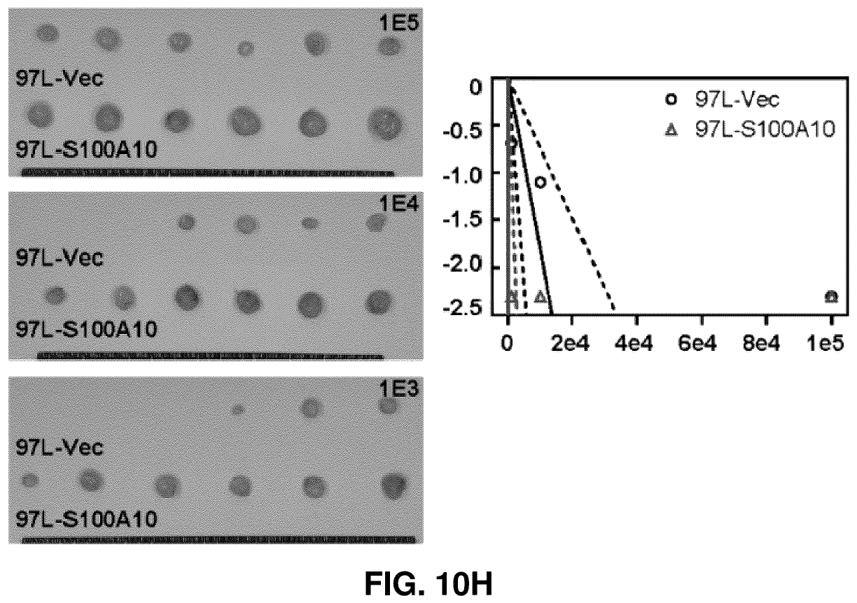

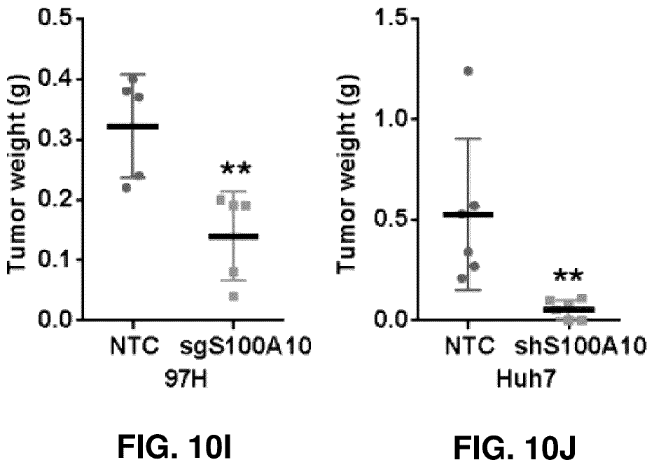

As S100A10 is specifically expressed in liver progenitor cells and premature hepatocytes stages, it was hypothesized that S100A10 might regulate HCC stemness. To investigate the functions of S100A10, S100A10 was stably overexpressed in an immortalized normal liver cell line MIHA and two HCC cell lines PLC/PRF/5 (PLC) and MHCC97L (97L) (authenticated to have no contamination; FIGS. 2A-2J, and Table 3), knocked down in Huh7 cells, and knocked out in MHCC97H (97H) cells using the CRISPR/Cas9 system (FIG. 2A). Over-expression of S100A10 increased the expression of stemness-related genes including CD24, CD44, LGR5, SOX2 and C-MYC in both HCC cell lines PLC and 97L (FIG. 2B and FIG. 10A). Focus formation ability was upregulated when S100A10 was upregulated, and was downregulated when S100A10 was knocked out or knocked down (FIGS. 10B-10F). Overexpression of S100A10 promoted the sphere-forming ability, while S100A10 knockout (KO) significantly suppressed the sphere formation ability of HCC and MIHA cells (FIGS. 2C-2E and FIG. 10G), indicating that S100A10 enhances self-renewal ability of HCC. To further investigate the in vivo tumorigenic ability of S100A10-overexpressing (OE) cells, limiting dilution assays performed in nude mice showed significantly higher tumor incidence and tumour growth rate in S100A10-OE cells in both PLC and 97L cell lines (FIG. 2F, Table 3, and FIG. 10H). In addition, S100A10 knockdown (KD) Huh7 and S100A10 KO 97 H cells were subcutaneously injected into dorsal flanks of nude mice, and the tumour volume was significantly smaller compared with non-target control (NTC) cells (FIGS. 10I and 10J). These results indicate that S100A10 promotes the tumorigenicity of HCC cells both in vitro and in vivo.

TABLE 3
The tumor-initiating frequency summarized in table form.

| No. of Cells injected with grow | | |
|---|---|---|
| factor reduced Matrigel (50%) | PLC-Vec | PLC-S100A10 |
| 1 × 10⁵ | 5/6 | 6/6 |
| 1 × 10⁴ | 4/6 | 6/6 |
| 1 × 10³ | 3/6 | 5/6 |
| Stem Cell Frequency | 1/19928 | 1/559 |
| CI (95%) | 1/55839-1/7112 | 1/1515-1/206 |
| P-value | | 1.1E−7 |

S100A10 Enhances the Chemoresistance of HCC In Vitro and In Vivo

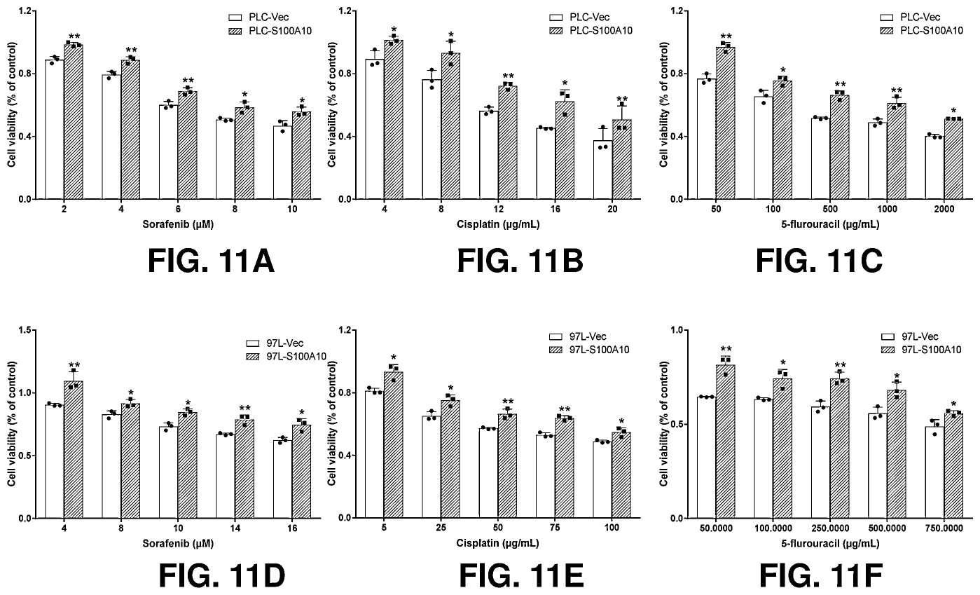

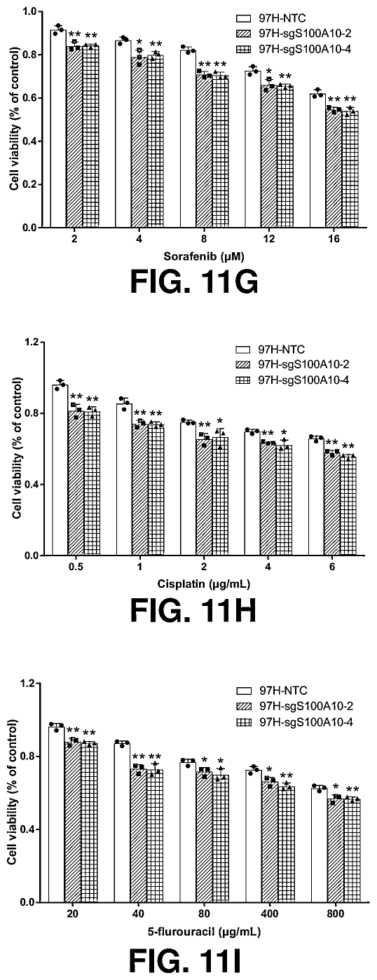

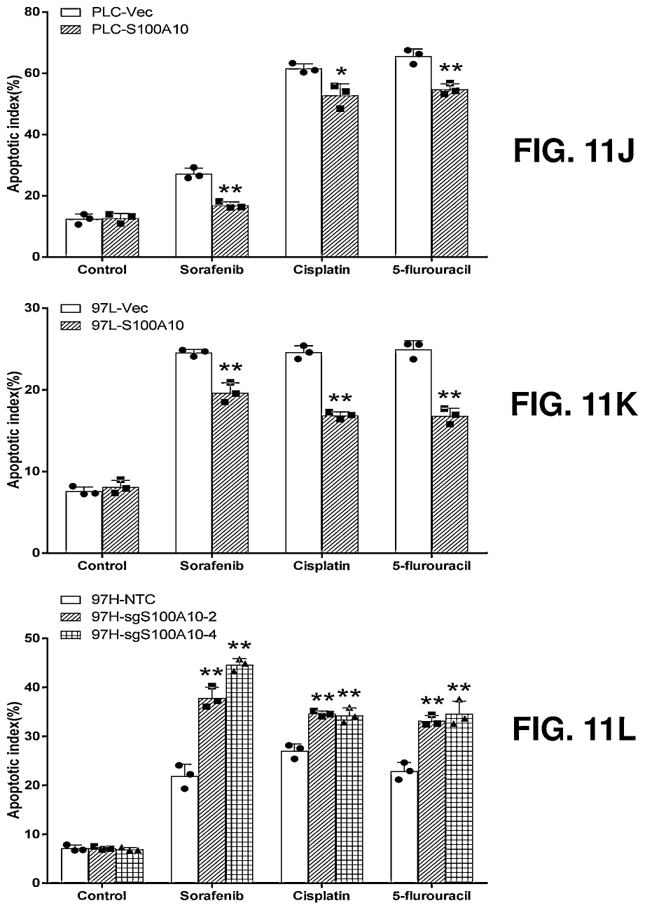

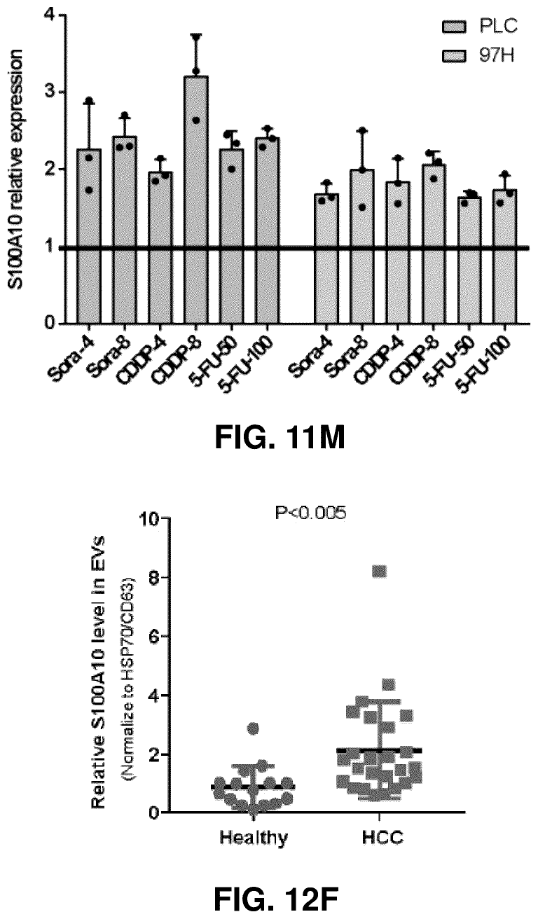

Chemoresistance is one of the important sternness-related characteristics of tumor cells. On treatment with chemotherapy drug sorafenib (alternative first-line drug for advanced HCC), cisplatin or 5-flurouracil (5-FU), the cell viability of S100A10-OE cells (PLC and 97L) were significantly higher, and the apoptotic indices were lower than those of the controls (FIGS. 11A-11L). Consistently, opposite results were observed in S100A10-KO 97 H cells (FIGS. 11A-11L).

We also confirmed that S100A10 enhanced the chemoresistance of HCC in vivo in nude mice with xenograft tumors induced by PLC-S100A10 cells. When treated with sorafenib, cisplatin or 5-FU, the xenograft tumors in the PLC-S100A10 group grew significantly larger than the control group (FIGS. 2E-2I). These results were consistent with those of previous report that S100A10 was related to multidrug resistance.¹⁵ Our qRT-PCR results also showed that S100A10 upregulated the expression of ABCG2, which is known to exert multidrug resistance (FIG. 2B and FIG. 10A).

In addition, activation of AKT and ERK has been implicated in stemness and sorafenib resistance in HCC.²²⁻²⁵ To this end, western blotting showed that the activation of AKT and ERK was significantly enhanced in S100A10-OE cells and reduced in S100A10-KO 97 H cells (FIG. 2J). Previously, S100A10 was found to be upregulated on chemotherapy treatment in breast cancer via HIF-1.²⁶ To investigate if this also held true in HCC, HCC cells were treated with chemotherapeutic agents and found that sorafenib, cisplatin and 5-FU all upregulated the expression of S100A10 and HIF-1α, indicating that S100A10 can also be upregulated by chemotherapeutic agents in relation to HIF-1 in HCC (FIG. 11M).

S100A10 Promotes HCC Metastasis In Vitro and In Vivo, Enhancing Epithelial-Mesenchymal Transition (EMT)

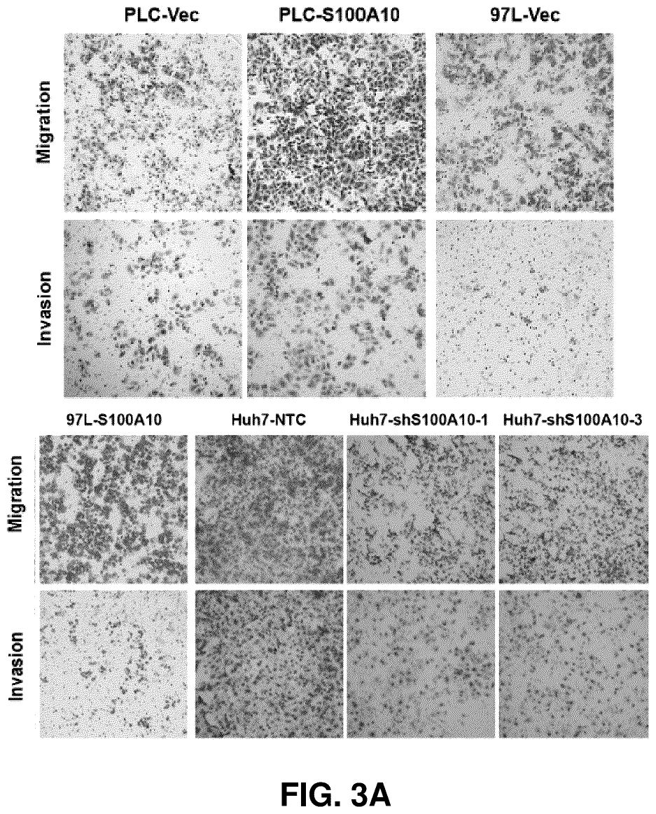

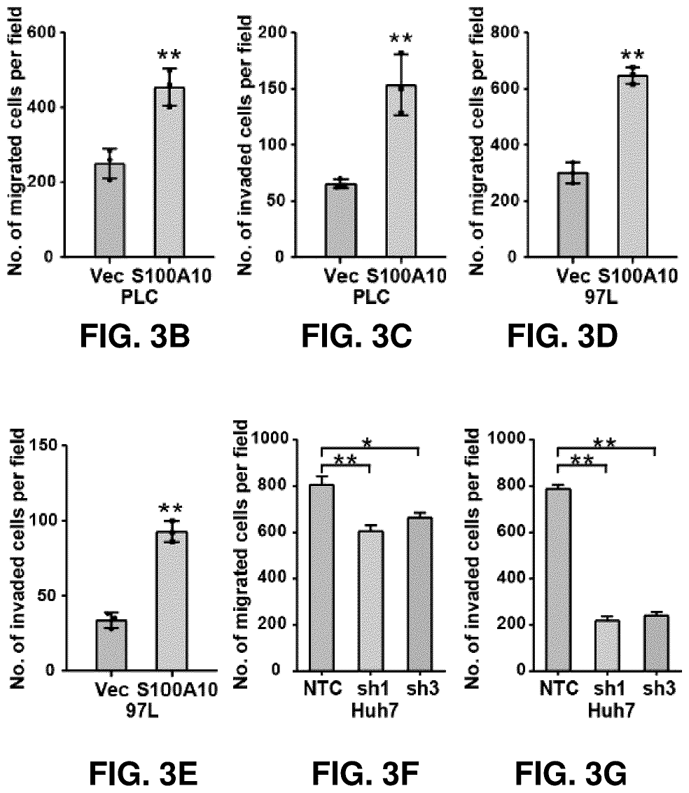

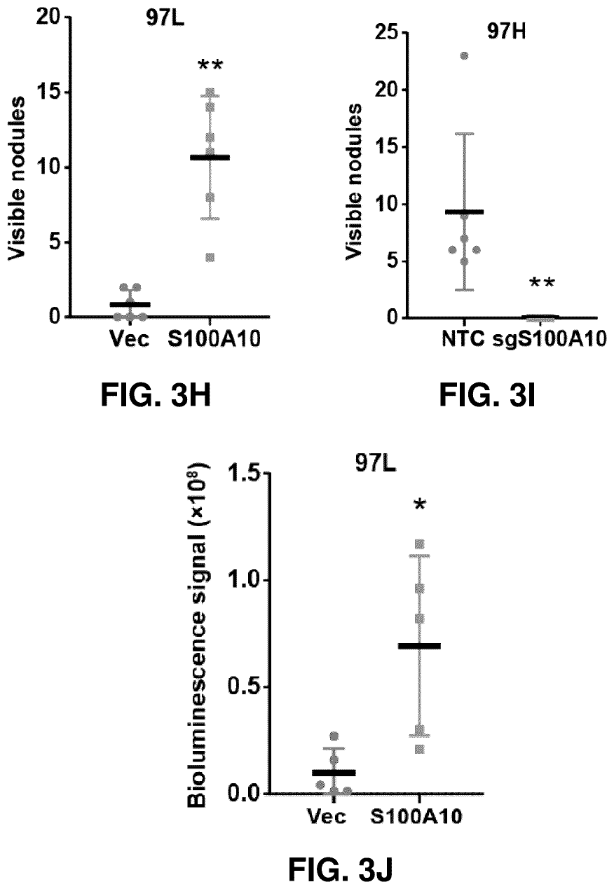

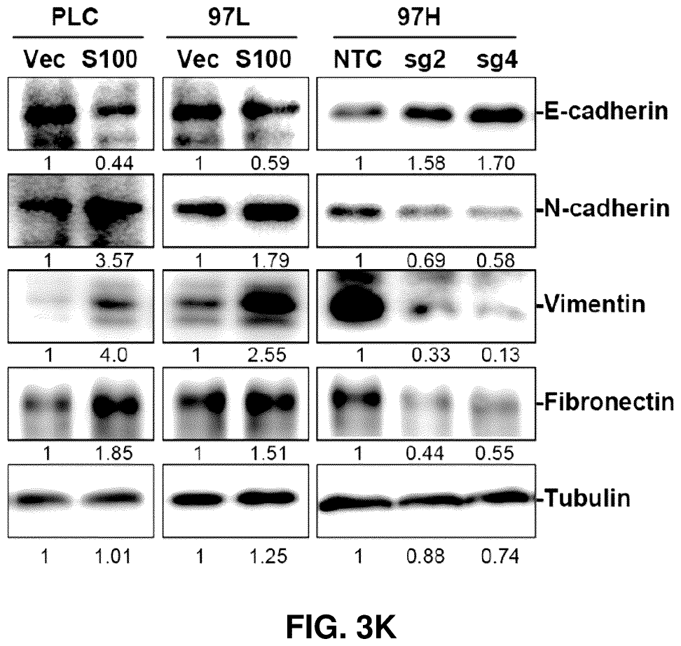

Cell migration and invasion assays revealed that S100A10-OE in HCC cell lines significantly enhanced cell migration and invasion (FIGS. 3A-3G), whereas silencing S100A10 significantly suppressed these abilities (FIGS. 3A-3G). We used two mouse models to assess metastasis: (1) liver metastasis model by intrasplenic injection and (2) lung metastasis model by tail vein injection. In the liver metastasis (intrasplenic injection) model, 10 weeks after intra-splenic injection, all mice injected with S100A10-OE 97L cells had metastatic nodules on their liver surfaces, whereas much fewer and smaller metastatic nodules were observed in three of six mice in the control group (FIG. 3H). Along the same direction, S100A10 KO in 97 H cells significantly inhibited its metastasis to the liver in this intrasplenic injection model (FIG. 3I). In the lung metastasis (tail vein injection) model, 8 weeks after tail vein injection, significantly higher luciferase signals were detected in S100A10-OE 97L cells as compared with control cells (FIG. 3J). Histology further confirmed the liver and lung metastatic lesions, respectively (FIGS. 3H-3K).

As EMT is well known to contribute to tumor metastasis, with western blotting, we found that S100A10 upregulated the expression of mesenchymal markers (N-cadherin, fibronectin and vimentin) and downregulated the expression of epithelial marker E-cadherin (FIG. 3E). Taken together, these findings showed that S100A10 promotes HCC liver and lung metastases, likely via the promotion of EMT.

S100A10 is Present in HCC-Derived EVs

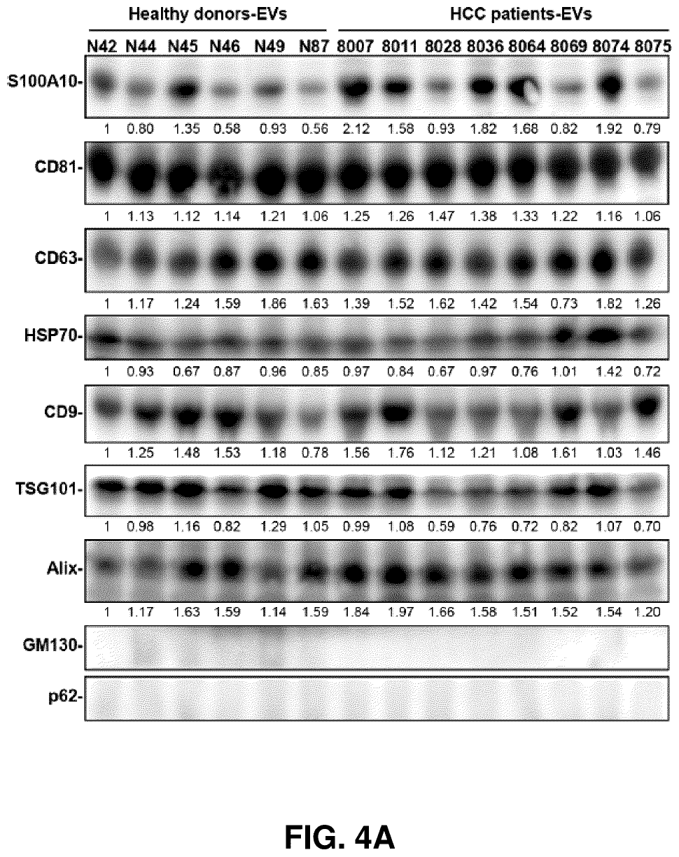

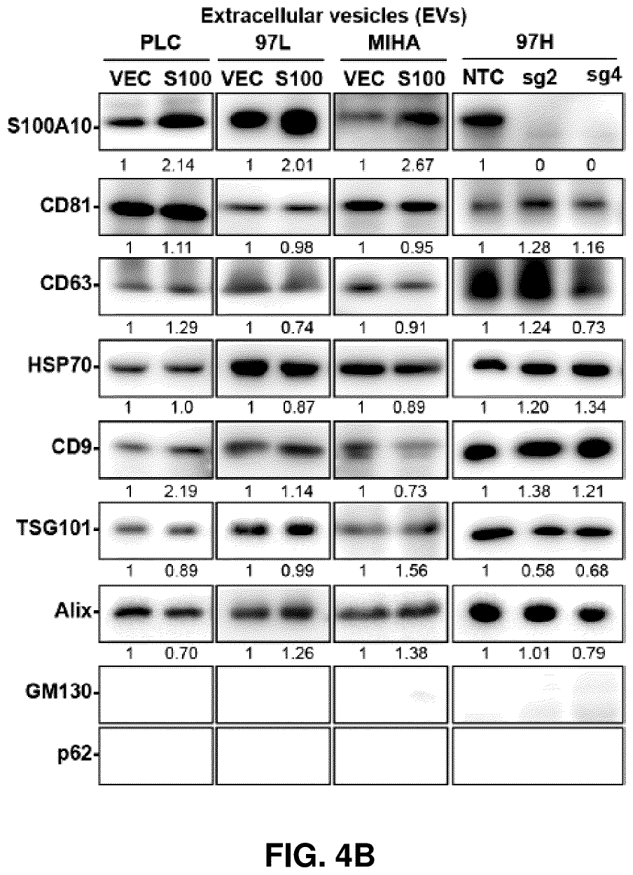

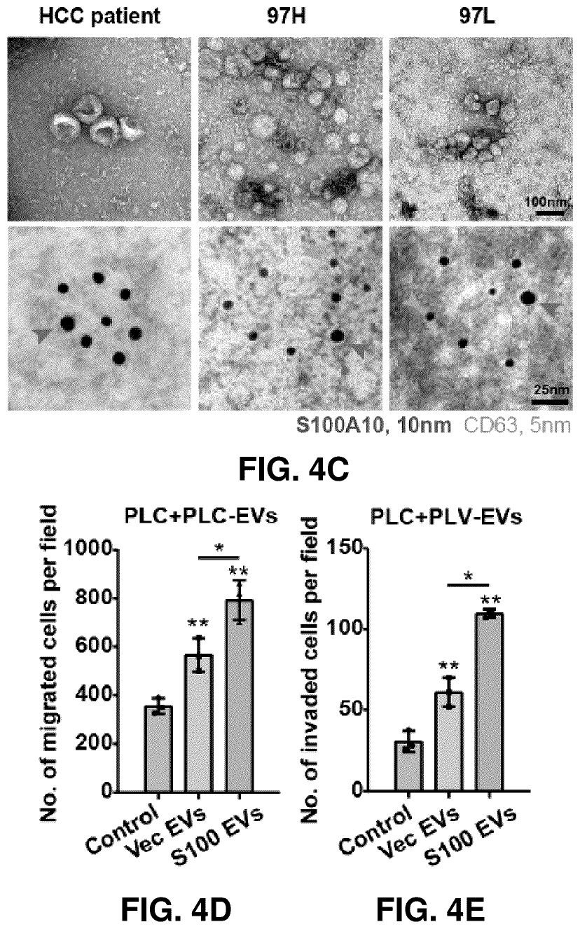

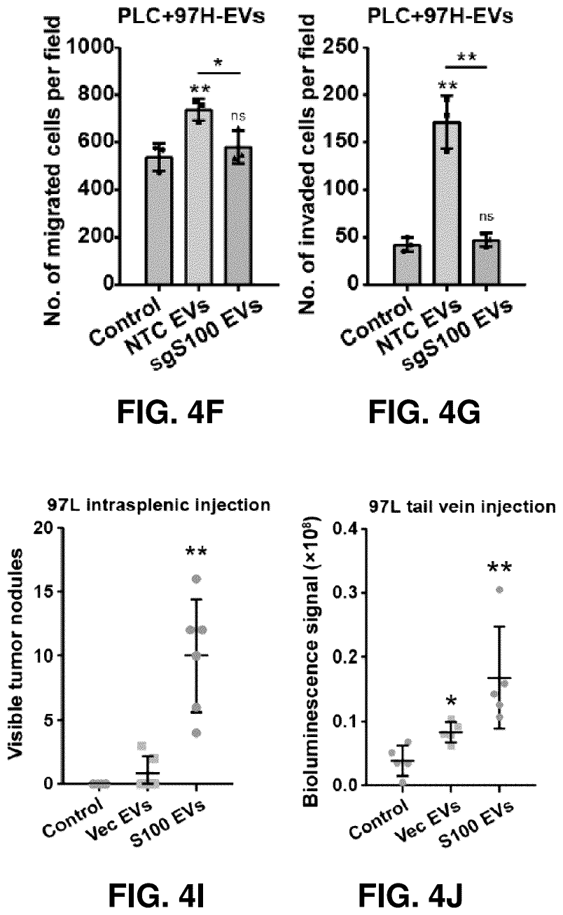

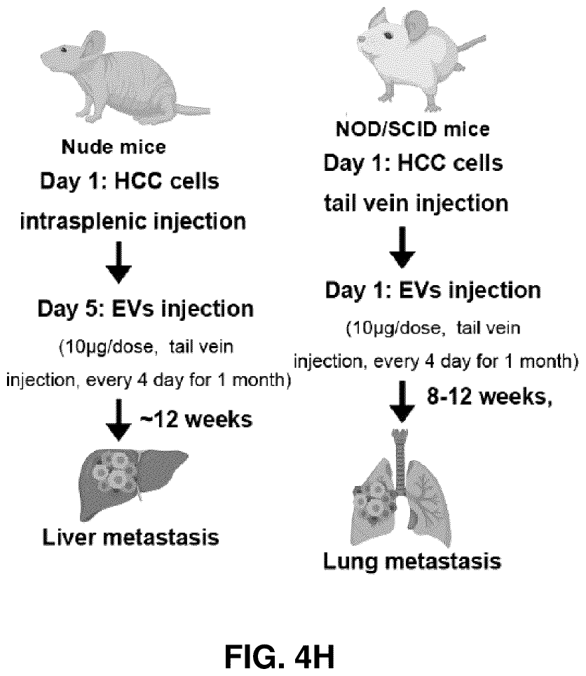

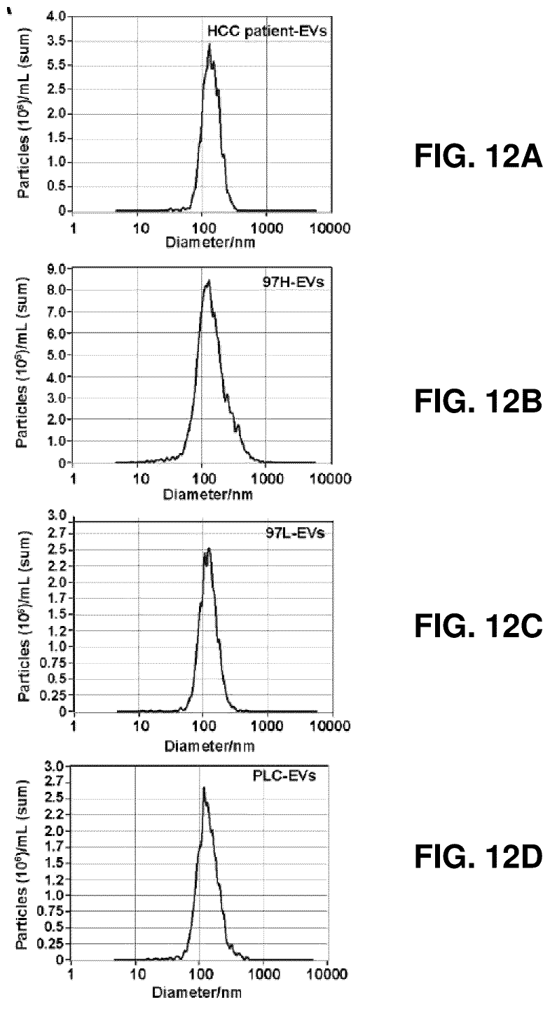

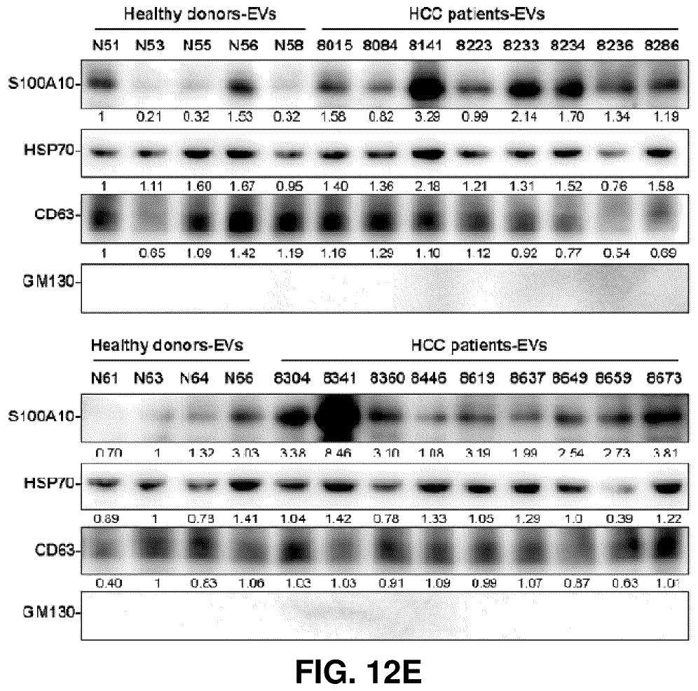

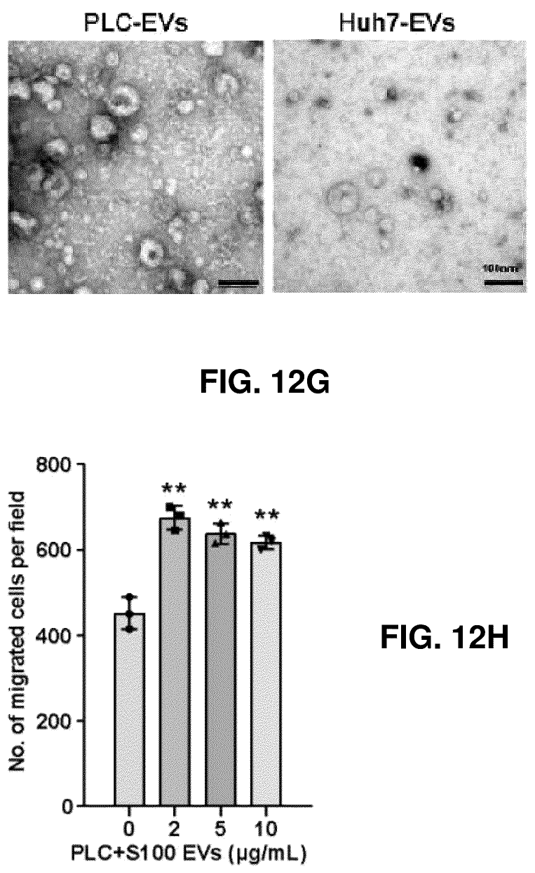

Although S100A10 has been predicted to be secreted into extra-cellular space, there have been no reports of such secretion in HCC. Furthermore, there are no reports of its transport into EVs. To investigate these queries, we isolated and characterized the EVs extracted from the plasma samples of 25 patients with HCC and 15 healthy subjects and HCC cell lines. The EVs were enriched by sequential centrifugation of plasma or supernatant through increasing gravitational forces to remove cellular debris and apoptotic bodies, before finally pelleting at 100 000 gravitational forces. Nanoparticle tracking analysis confirmed the extracted EVs had a size with peak around 120-140 nm (FIGS. 12A-12D); western blotting also confirmed the presence of EVs with EV-specific markers (including CD81, CD63, HSP70, CD9, TSG101 and Alix) and EV-negative markers (GM130 and p62) (FIGS. 3A-4B and FIGS. 12E and 12F). In addition, on transmission electronic microscopy, we identified the EVs with a cup-shaped morphology and a diameter around 30-160 nm (FIG. 4C and FIG. 12G) Immunogold labelling further revealed the presence of S100A10 on the surface of CD63-positive EVs derived from both plasma and HCC cells of patients with HCC (FIG. 4C).

In the S100A10-OE HCC cells, the level of S100A10 carried in EVs (termed EV-S100A10 and thereafter) was also upregulated, whereas in S100A10-KD and S100A10-KO HCC cells, the EV-S100A10 level was consistently downregulated or not detectable (FIG. 4B). Western blotting also suggested that EVs derived from plasma samples of patients with HCC exhibited a higher level of S100A10 as compared with those from healthy subject plasma (FIG. 4A and FIGS. 12E and 12F). All these data indicate that S100A10 is secreted and carried in EVs.

S100A10-Enriched EVs Promote HCC Metastasis

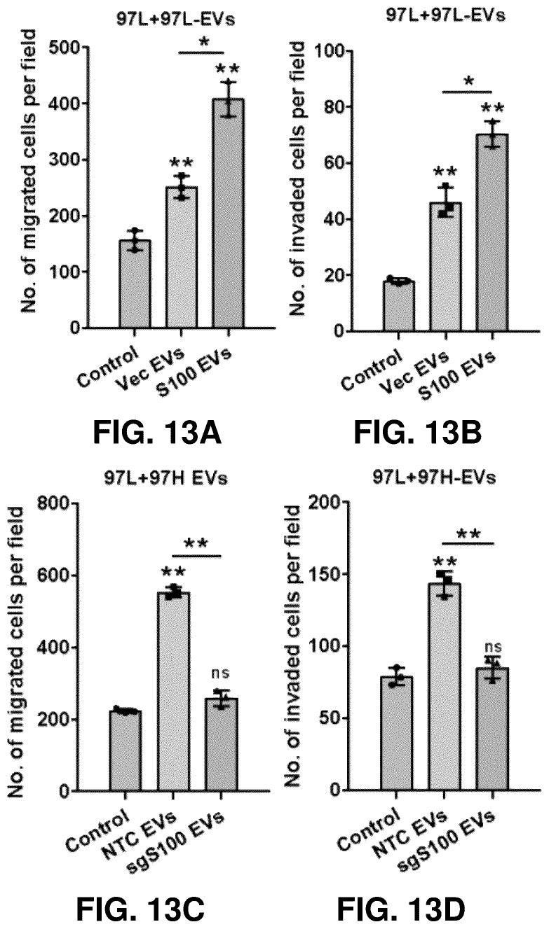

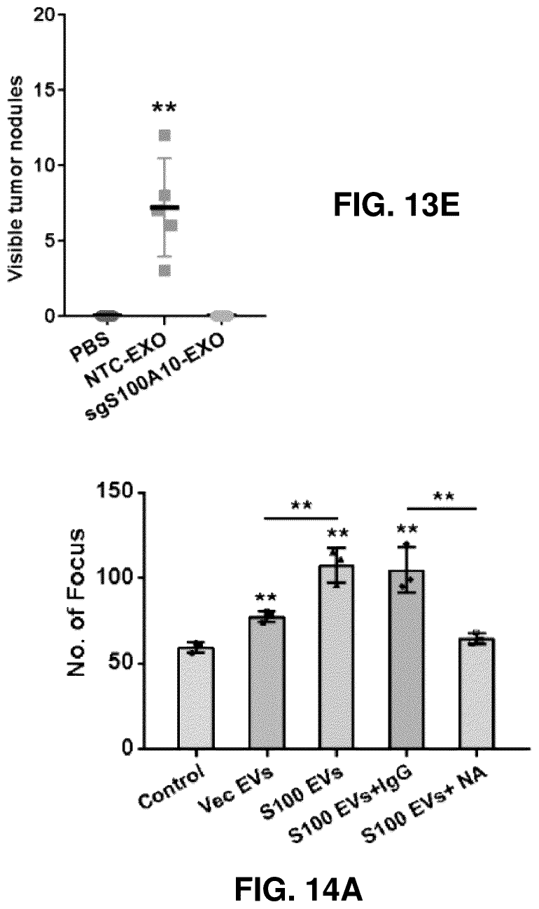

To investigate whether S100A10-enriched EVs promoted HCC cell motility, different concentrations of EVs derived from S100A10-OE cells (S100A10 EVs) were used to pretreat HCC cells for 48 hours. From the migratory assay, treatment with 2 μg/mL S100A10 EVs was already potent enough to significantly promote PLC cell migration (FIG. 12H). Furthermore, compared with phosphate buffered saline (PBS)-treated cells, both S100A10 EVs and extracellular vesicle from vector control cell (Vec EVs) (EVs derived from vector control (Vec) cells) promoted HCC cell migration and invasion, but S100A10 EVs exhibited a stronger promoting ability than the Vec EVs (FIGS. 4D-4G; FIGS. 13A-13D). On the other hand, we queried whether EVs derived from different HCC cell lines might exert similar effects on HCC cells; EVs derived from S100A10-KO 97H (sgS100 EVs) or NTC control (NTC EVs) were used to treat PLC or 97L cells, and the cell migration and invasion assays were performed. The results showed that NTC EVs significantly promoted the migratory and invasive abilities, while KO of S100A10 abrogated these effects (FIG. 4D-4G and FIGS. 13A-13D). These findings indicate EVs with S100A10 exhibit remarkable effects on HCC cell motility.

Furthermore, we employed two EV education mouse models to further evaluate the effects of S100A10 EVs in facilitating HCC liver or lung metastasis (liver metastasis model by intra-splenic injection and lung metastasis model by tail vein injection). First, nude mice intrasplenically injected with 97L or 97H HCC cells were given intravenous injection of EVs derived from S100A10-OE or S100A10-KO HCC cells every 4 days for 4 weeks to study the metastatic ability to the livers (FIG. 4H, left panel). On treatment with 97L-S100A10-EVs, the 97L liver metastatic nodules were markedly increased in both size and number when compared with treatment with 97L-Vec- EVs or PBS (FIG. 4I). In contrast, in mice treated with EVs derived from S100A10-KO 97 H cells, there was no increase in the liver metastasis, whereas the 97H-NTC-EVs enhanced the liver meta-static ability compared with PBS control (FIG. 13E).

Second, NOD SCID mice intravenously injected (via tail veins) with luciferase-labelled 97L HCC cells were similarly treated with S100A10 EVs to evaluate the metastatic ability to the lungs (FIG. 4H, right panel). Similarly, in the lung metastasis model using tail vein injection, the luciferase signal was significantly enhanced by EVs derived from S100A10-OE cells, and the luciferase signal was much stronger in S100A10 EV-treated mice (FIG. 4J). The histology also supported the observations.

EV-S100A10 is Functional and Promotes HCC Sternness, which is Abrogated with NA

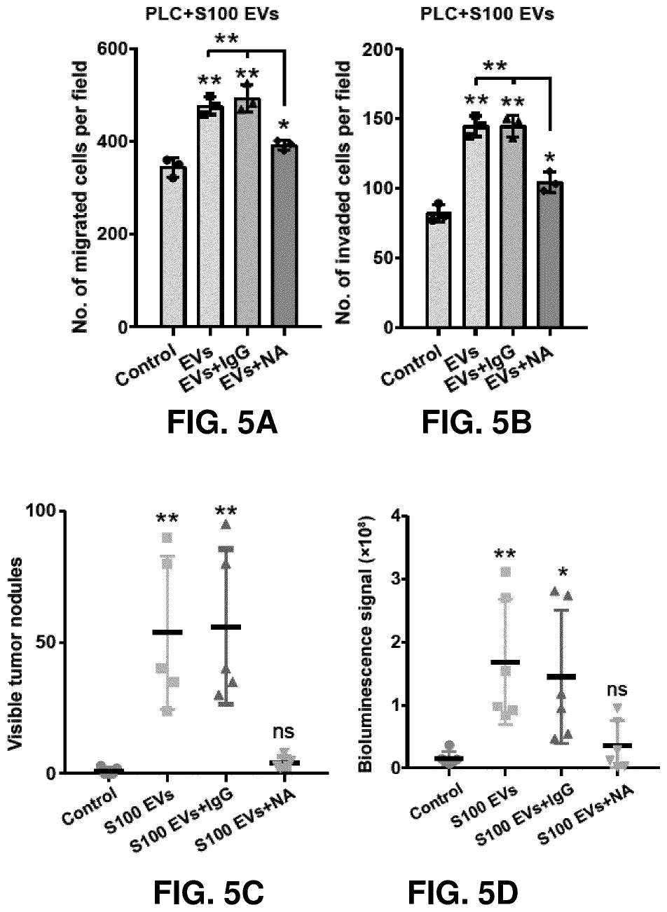

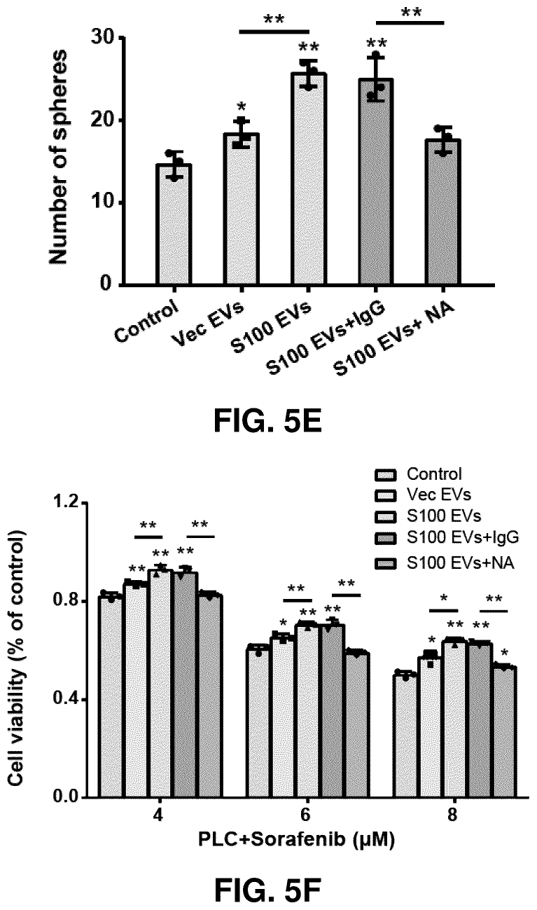

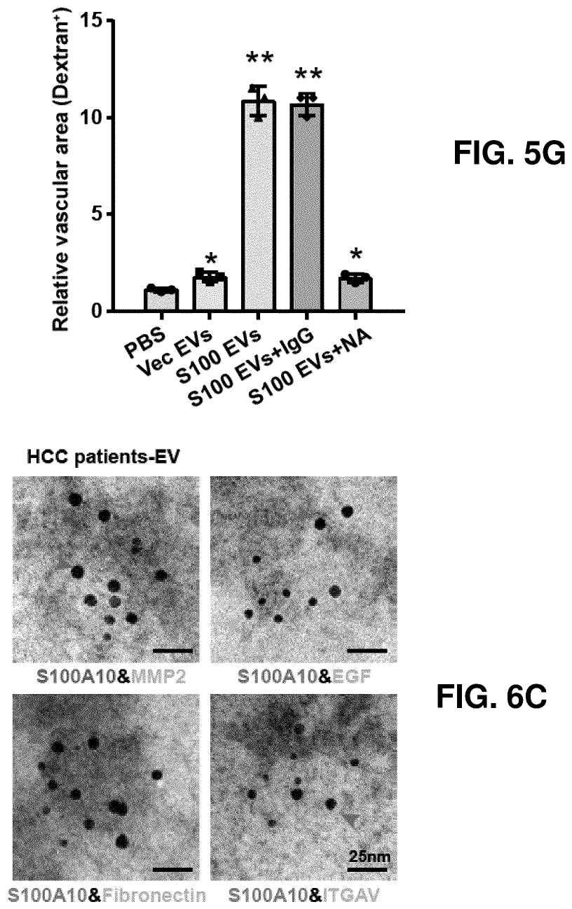

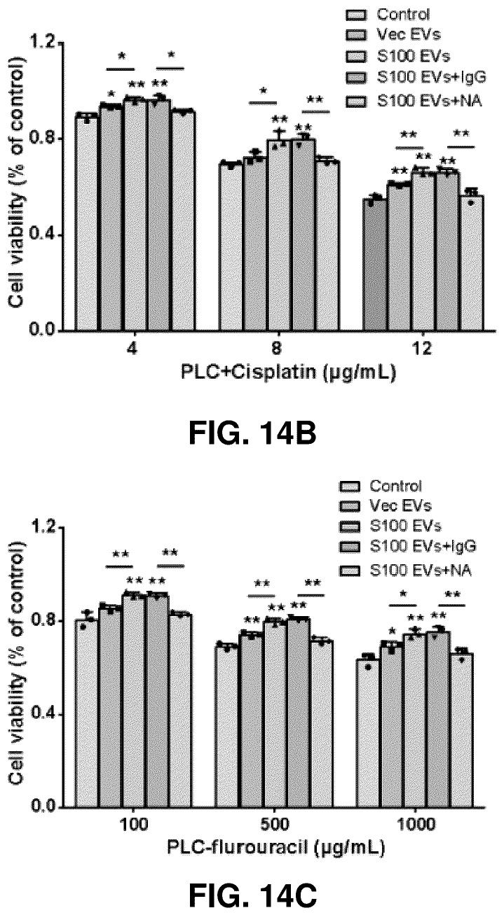

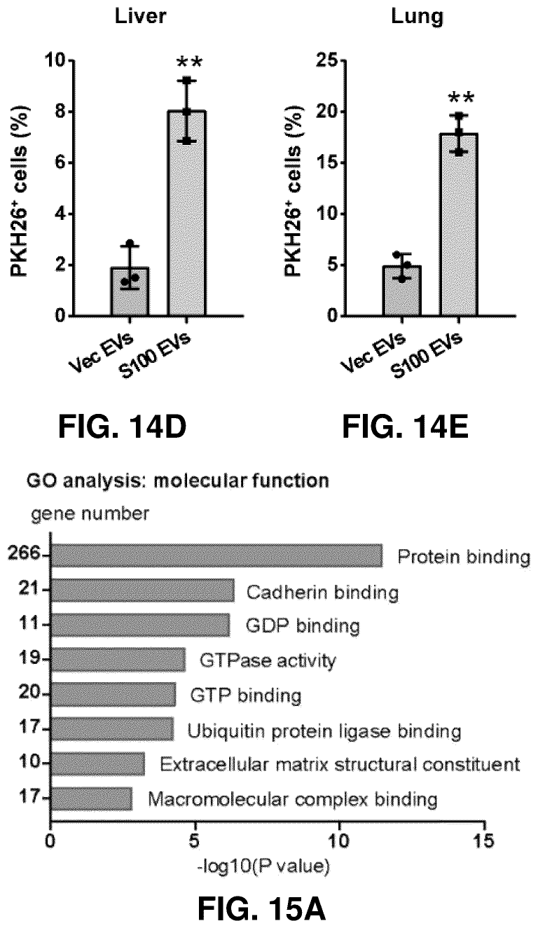

To investigate the underlying mechanism by which S100A10-enriched EVs enhanced HCC development, we employed NA against S100A10 to see if it was able to block the effects of S100A10 EVs in promoting cancer stemness. When PLC-S100A10-EVs were preincubated with the NA and then applied to HCC cells as pretreatment for migration and invasion assays, the enhanced migratory and invasive abilities of PLC cells were markedly abrogated (FIGS. 5A and 5B). Furthermore, the in vivo meta-static ability of 97L cells induced by S100A10 EVs was mark-edly abrogated by coinjection with S100A10 NA in both liver metastasis and lung metastasis mouse models (FIGS. 5C and 5D). The histology confirmed the observations. In addition, cotreatment with S100A10 NA abol-ished the focus formation, sphere formation and chemore-sistance enhanced by S100A10 EVs (FIGS. 5E and 5F, and FIGS. 14A-14C). Altogether, these findings demon-strated that S100A10 in EVs plays a key role in promoting HCC stemness features and indicate that S100A10 NA is a potential therapeutic option for HCC.

EV-S100A10 Affects the Distribution of EVs and Promotes Pulmonary Leakiness

Various studies revealed that EVs can prime a supportive micro-environment to facilitate metastasis. To investigate the distribution and uptake of EVs in different organs, EVs derived from 97L-Vec or S100A10-OE were labelled with PKH26 and injected intravenously into mice. The results showed that EVs predominantly localized in the lungs and livers, and more S100A10 EVs were observed compared with Vec EVs in these organ tissues (FIGS. 14D and 14E).

As destabilization and increased vascular permeability in the lungs are early events in premetastatic niche formation,20 we then performed pulmonary leakiness assay. The result showed that S100A10 EVs enhanced the pulmonary endothelial permeability in mice when compared with PBS control and Vec EVs, as indicated by the larger area of dextran staining (FIG. 5G). When S100A10 EVs were injected together with S100A10 NA, the effect was significantly abolished (FIG. 5G).

S100A10 Alters the Protein Cargos of EVs

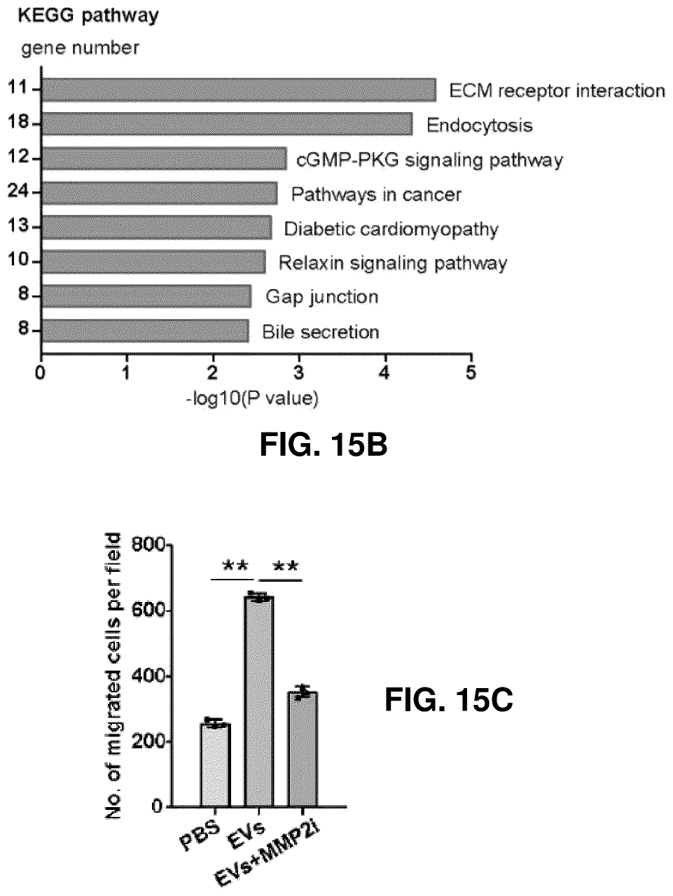

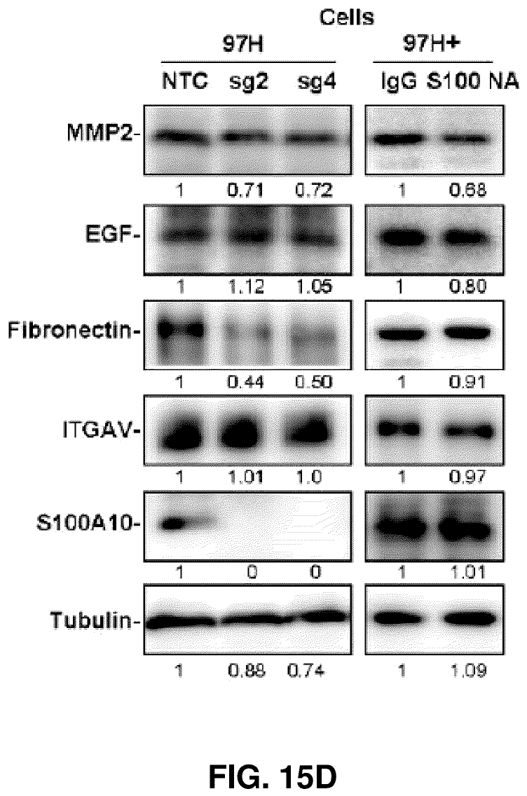

Mass spectrometry was performed to compare the different proteins in 97L-Vec-EVs and 97L-S100A10-EVs. 364 proteins were found to be significantly upregulated more than 1.5 folds in S100A10-enriched EVs. Gene ontology (GO) analysis revealed that most of the upregulated proteins were involved in the protein binding and ECM constituent (FIGS. 15A and 15B). KEGG enrichment analysis showed that the upregulated proteins mainly participated in ECM receptor interaction, endocytosis and pathways in cancer (FIGS. 15A and 15B).

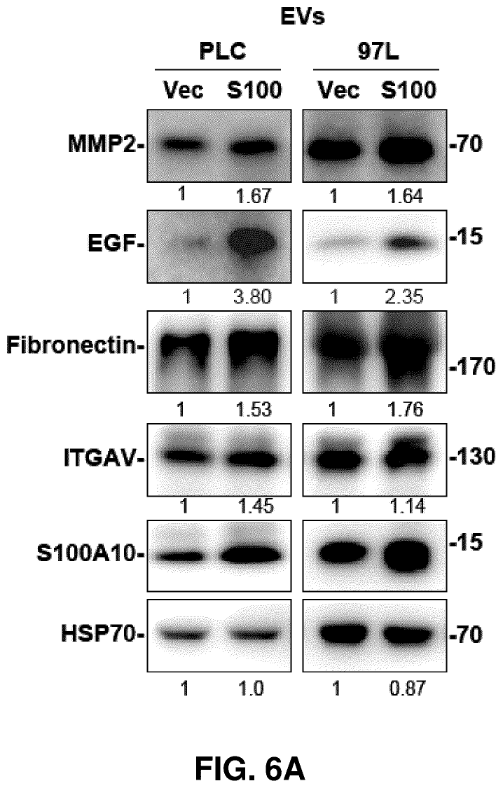

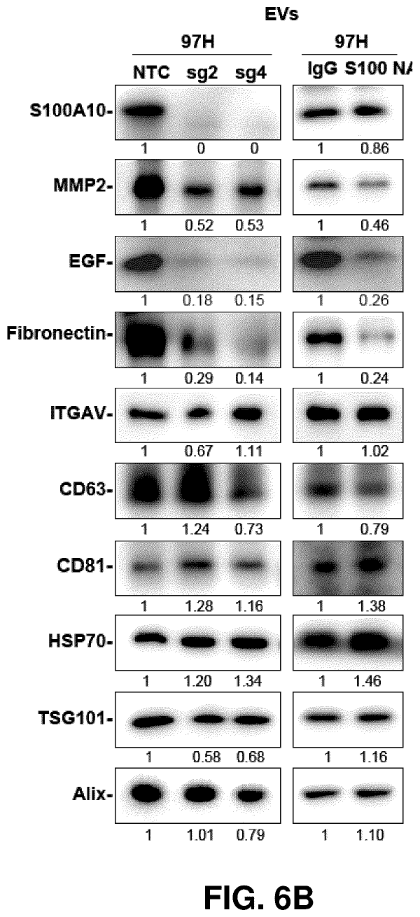

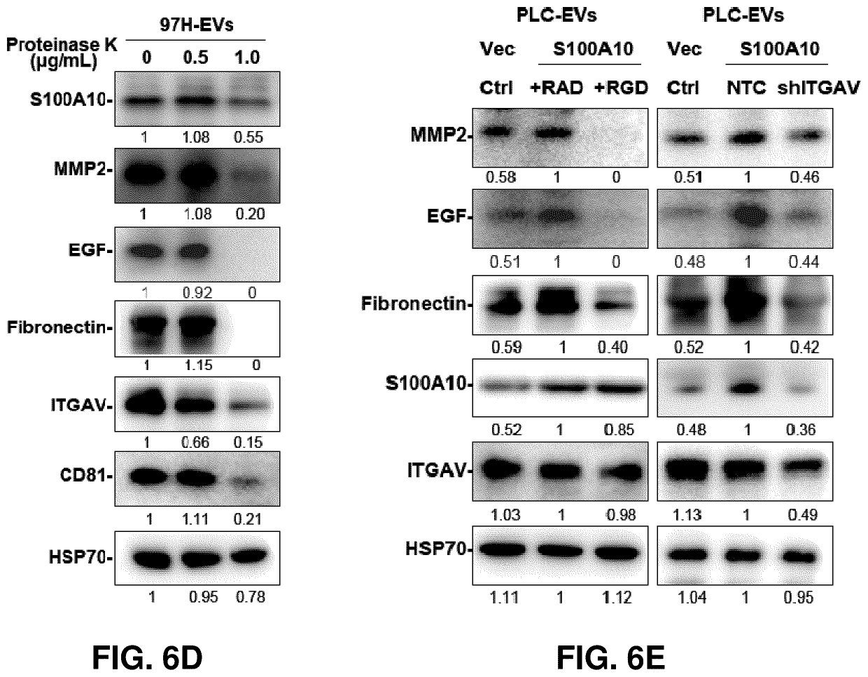

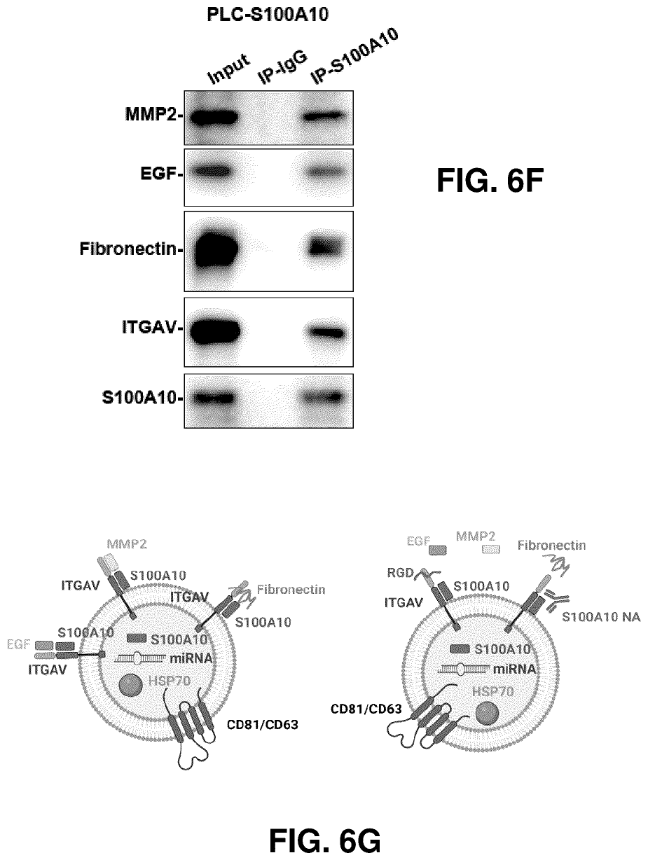

From the results of GO and KEGG analyses, MMP2 and fibronectin were enriched in more than one of these processes. As a plasminogen receptor, S100A10 is important in accelerating the degradation of ECM such as fibronectin, activation and secretion of MMPs and growth factors. Therefore, we focused mainly on MMP2 and fibronectin, and determined if growth factors such as EGF and HGF in EVs were affected by S100A10. Western blotting verified that OE of S100A10 increased the MMP2, fibronectin and EGF levels in the EVs (FIG. 6A), although EGF was not detected in the mass spectrometry results, whereas KO of S100A10 decreased the levels of the MMP2, fibronectin and EGF in the EVs (FIG. 6B). Recent study has shown that cytokines in the tumor microenvironment could bind to the surface of EVs secreted by cancer cells. 27 We next examined whether MMP2, fibronectin and EGF were localized within EVs or present on the surface of EVs. First, our immunogold labelling indicated that MMP2, fibronectin and EGF were present on the membrane of EVs together with S100A10 (FIG. 6C). Then, we treated 97H-EVs with proteinase K to degrade proteins at a concentration that led to the degradation of the outer membrane proteins such as CD81, but not the intravesicular HSP70. The findings showed that the distribution of MMP2, fibronectin and EGF was limited to the EV surface, while S100A10 was localized both within and on the membrane of EVs (FIG. 6D). To assess if MMP2 carried by EVs was biologically activated, we performed gelatin zymography assay and found significant MMP2 activity of EVs derived from HCC cells. On the other hand, S100A10 EVs treated with MMP2 inhibitor abrogated the enhanced migratory ability of HCC cells by EVs (FIG. 15C). 28

S100A10 Mediates the Targeting of MMP2, EGF and Fibronectin to EV Membranes Through Integrin

To evaluate the role of S100A10 in regulating the binding of MMP2, fibronectin and EGF to the surface of EVs, we treated the 97 H cells with S100A10 NA before isolation of EVs and assessed the levels of MMP2, fibronectin and EGF of EVs. The results showed that, similar to S100A10-KO, when S100A10 was blocked, MMP2, fibronectin and EGF levels on EVs were significantly decreased, while that of S100A10 remained relatively unchanged (FIG. 6B). The expression levels of MMP2, fibronectin and EGF in 97H-S100A10-KO cells and 97 H cells treated with S100A10 NA or IgG were not reduced as significantly as those in the EVs (FIG. 15D). These results suggest that S100A10 may mainly affect the packaging of MMP2, fibronectin and EGF into EVs but not through affecting these proteins level in cells.

ITGAV has been reported to bind MMP2, EGF and fibronectin, and to be present on the surface on EVs. Using immunogold labelling and proteinase K treatment, we confirmed that ITGAV was present on the surface of EVs (FIGS. 6C and 6D). Previous study has shown that targeting of fibronectin to EVs depends on binding to integrins.29 To test whether S100A10 mediated the targeting of MMP2, fibronectin and EGF to EVs by binding to integrins, we inhibited the possible interaction with ITGAV by treating S100A10-OE cells with integrin ligand-mimetic peptide GRGDSP peptides (RGD). On treatment of PLC-S100A10 cells with the integrin-binding RGD peptide for 48 hours, the levels of MMP2, fibronectin and EGF on EVs were greatly reduced, but no changes were observed in S100A10, as compared with cells treated with the control GRADSP peptides (RAD) (FIG. 6E). Likewise, KD of ITGAV in S100A10-OE cells also decreased the MMP2, fibronectin and EGF levels in EVs (FIG. 6E). These data indicate that S100A10 mediates MMP2, EGF and fibronectin targeting to EVs through binding to integrin and then secreted in an adhesive form in EVs to promote the cell motility of recipient cells. Coimmunoprecipitation assay also revealed that S100A10 physically bound with ITGAV, MMP2, fibronectin and EGF (FIG. 6F). These findings suggest that S100A10 is an important mediator between integrin and specific secretory proteins targeting to EV, as represented by the schematic diagram (FIG. 6G).

S100A10 Enhances HCC Progression Through EGFR Activation

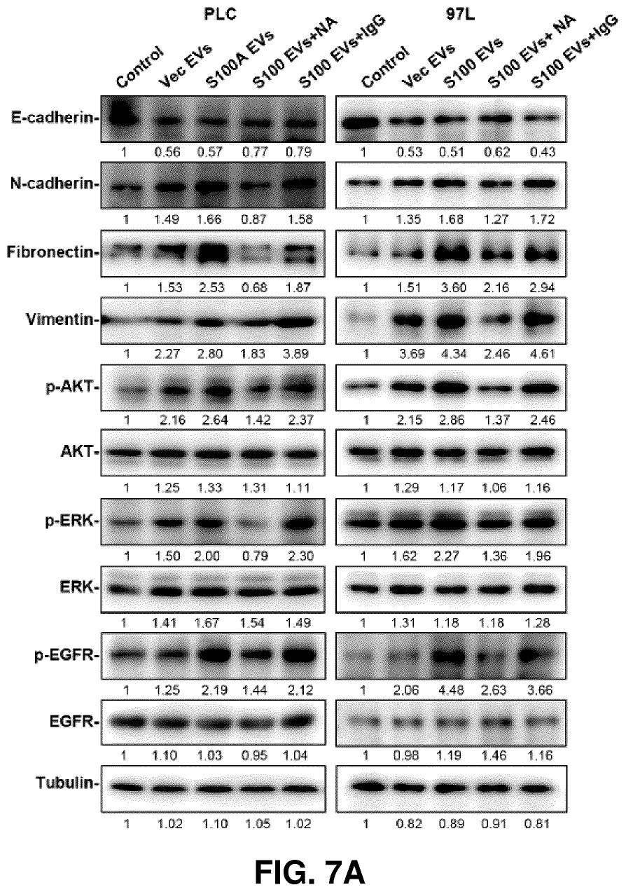

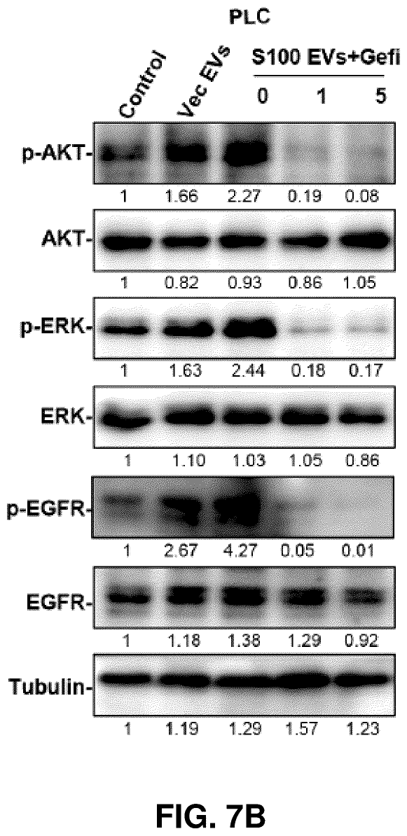

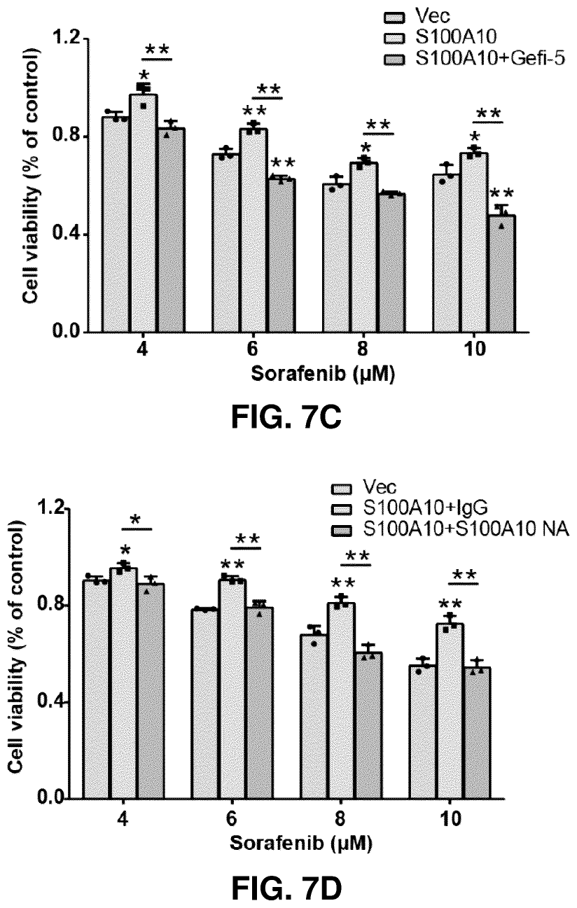

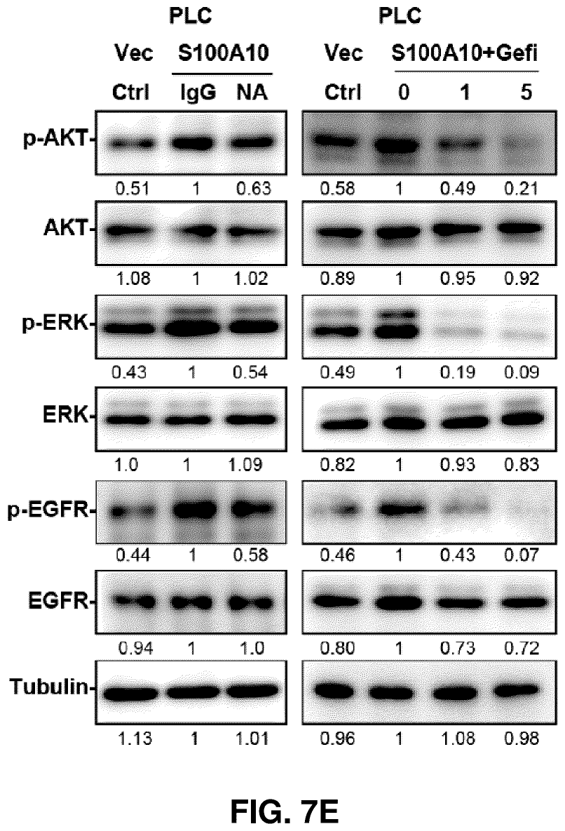

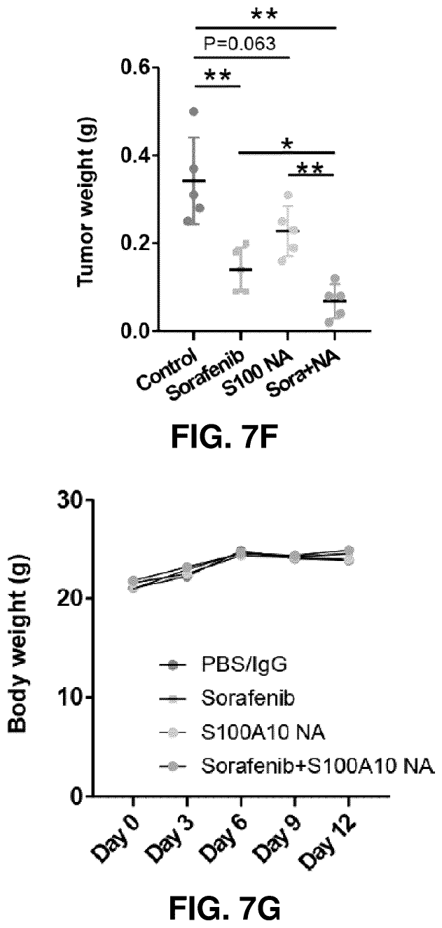

To further investigate the underlying mechanism how EV-S100A10 enhanced HCC metastasis, self-renewal and chemoresistance, we examined the possible signaling pathways involved. PLC-S100A10 EVs upregulated the expression of the mesenchymal markers (N-cadherin, fibronectin and vimentin) and downregulated that of epithelial marker E-cadherin (FIG. 7A), and S100A10 EVs might activate AKT and ERK with associated activation of EGFR (FIG. 7A). To further validate the importance of EGFR activation in this process, we used an EGFR inhib-itor, gefitinib, together with S100A10 EVs, to treat HCC cells, and the results showed that gefitinib significantly inhibited the activation of EGFR, AKT and ERK induced by EV-S100A10 (FIG. 7B).

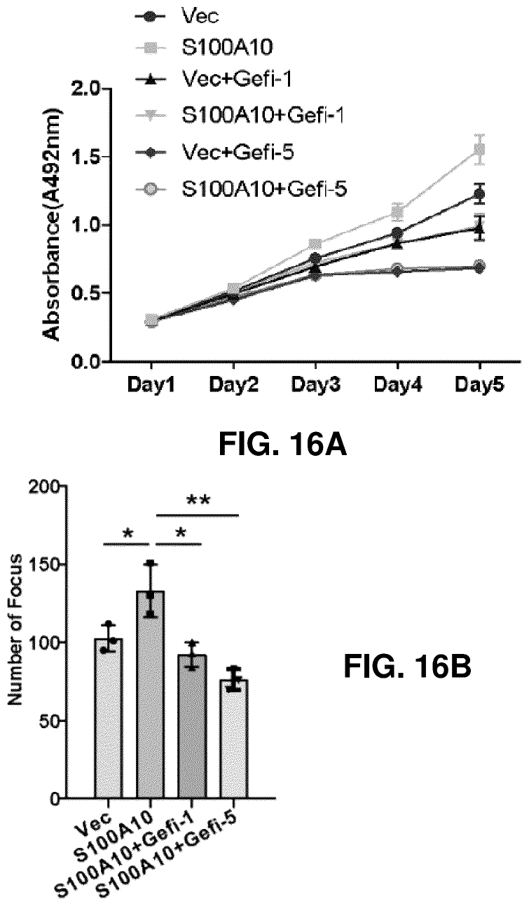

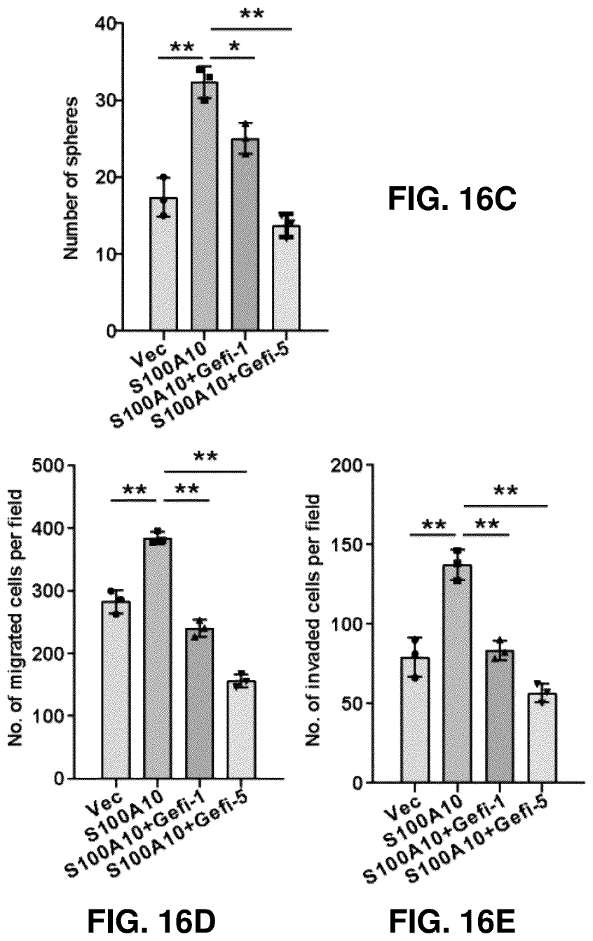

To further determine if the EGFR inhibitor gefitinib could decrease the oncogenic effects of S100A10, gefitinib was used to treat PLC S100A10-OE cells. First, XTT assay for cell proliferation showed that S100A10-OE cells were more sensitive to gefitinib than the Vec cells (FIG. 16A). Furthermore, gefitinib significantly suppressed focus formation, sphere formation, chemoresistance, migration and invasion ability of S100A10-OE cells (FIG. 7C and FIGS. 16B-16C). Moreover, when S100A10 NA was added to the culture medium of S100A10-OE cells, the NA treatment significantly decreased the chemoresistance to sorafenib (FIG. 7D). The treatment with both S100A10 NA and gefitinib suppressed the activation of EGFR, AKT and ERK, indicating that the oncogenic effect of S100A10 is dependent on the EGFR activation (FIG. 7E). With subcutaneous injection of the mouse model, we also found that S100A10 NA suppressed tumour growth and enhanced the antitumour efficiency of sorafenib in vivo (FIG. 7F). By monitoring the body weights of the mice that undergo different treatment, no significant changes were observed, and this implies no significant adverse effects of S100A10 NA in body organs in mice (FIG. 7G).

EV-S100A10 Exerts Chemotaxis on HCC Cells as a Potential Ligand of EGFR

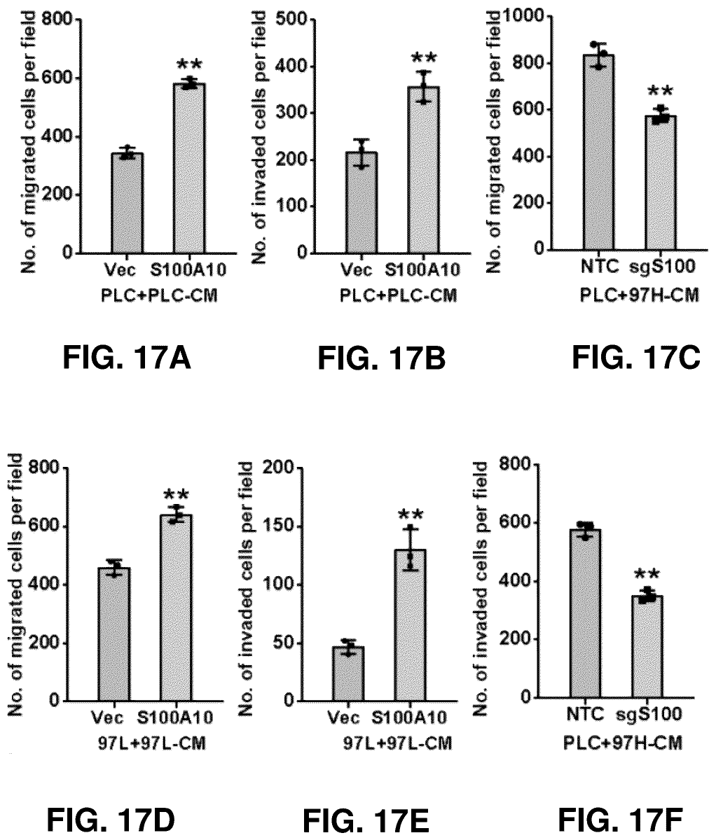

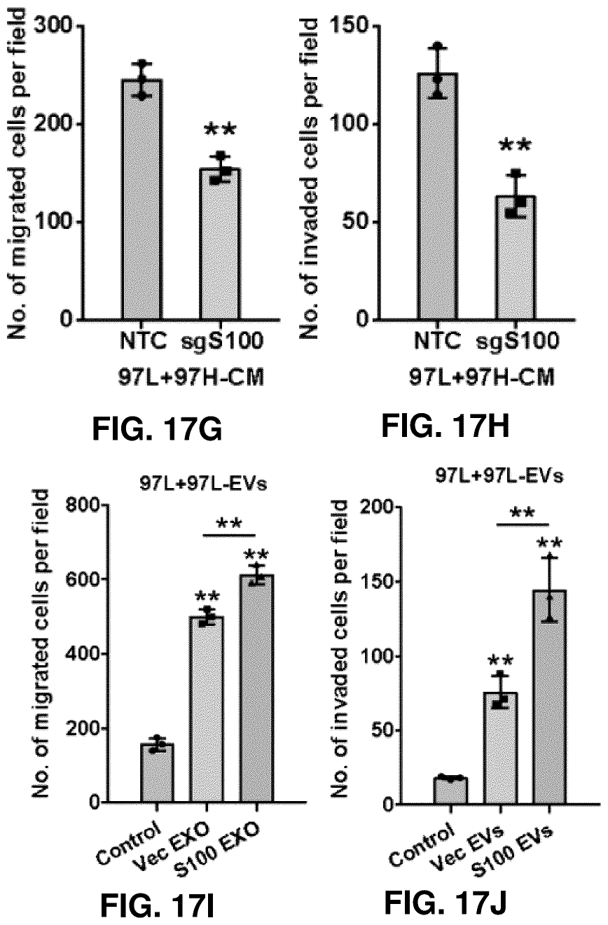

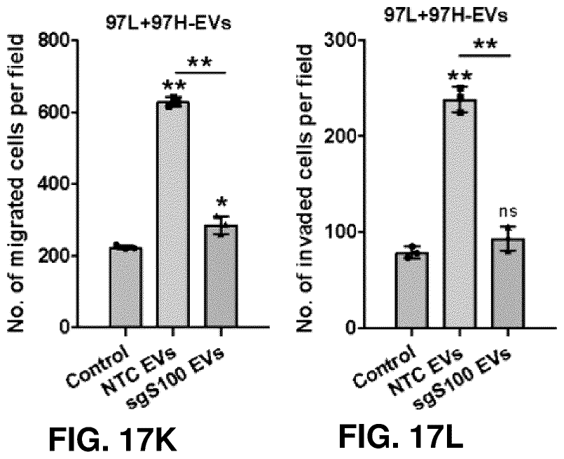

EVs have been reported to contain multiple cytokines/chemokines to exert chemotaxis on cancer cells.^{27 30-32} Extracellular S100 proteins have been reported to function in a cytokine-like manner via interaction with cell surface receptors.¹² However, whether EV-S100A10 has chemoattractive function is unknown. To this end, we applied condition medium (CM) from S100A10-OE or Vec cells in the lower compartment of the transwell chambers. The CM from S100A10-OE cells significantly enhanced the migratory and invasive abilities of the HCC cells (FIGS. 17A-17H). On the other hand, we queried whether CM derived from different cell lines might exert similar effects on HCC cells. We applied the CM derived from S100A10-KO 97H to PLC or 97L cells and performed the cell migration and invasion assays. The migratory and invasive abilities of PLC and 97L cells were significantly decreased with S100A10-KO 97H CM compared with the NTC-97H CM (FIGS. 17A-17H). These results suggest that the secreted biomolecules in CM regulated by S100A10 exert chemotactic effects on HCC cells.

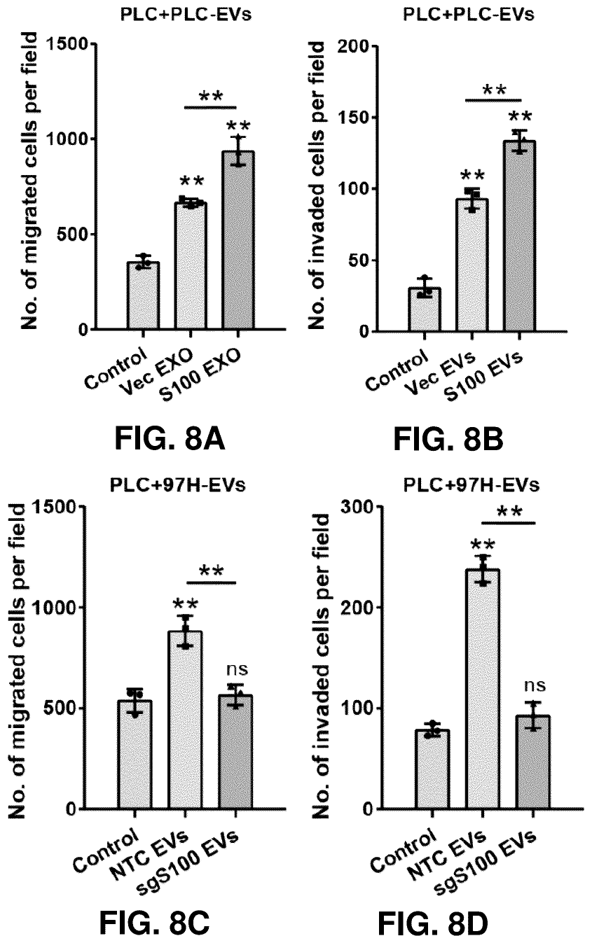

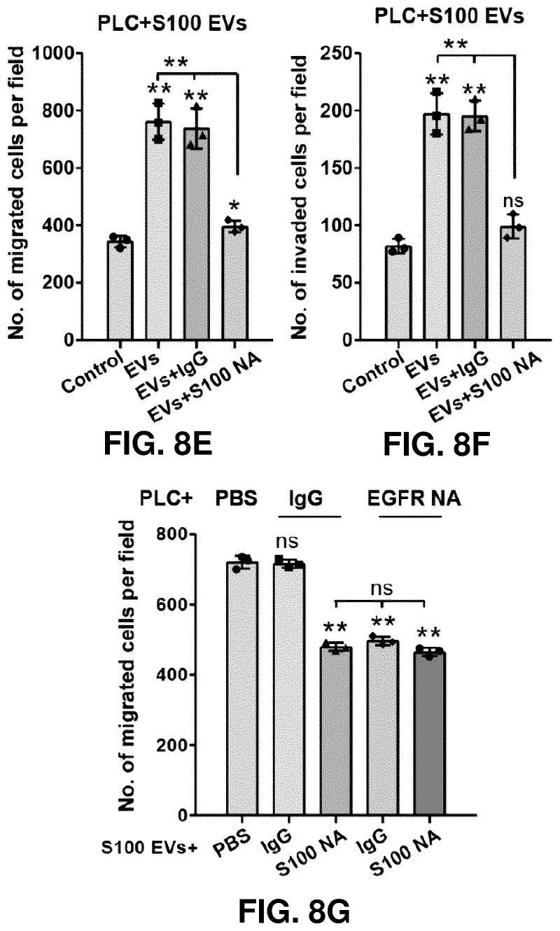

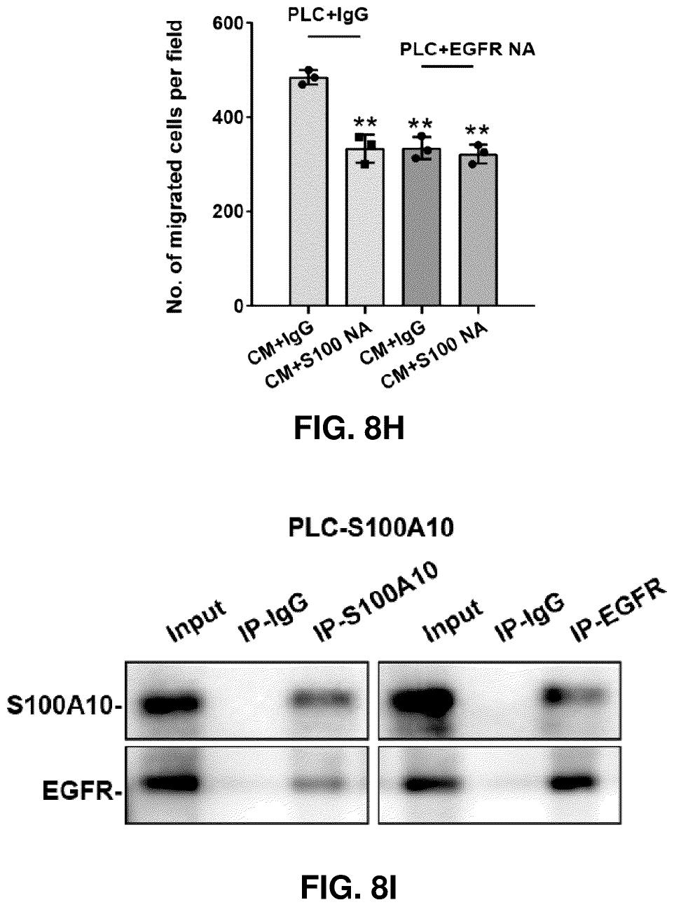

Furthermore, we added EVs, instead of CM, to the lower compartment of the transwell chambers. Addition of S100A10 EVs promoted the migratory and invasive abilities of HCC cells (FIG. 8A-8B and FIGS. 17I-17L), and S100A10 EVs exhibited a stronger effect as compared with Vec EVs. While 97H-NTC derived EVs promoted migration and invasion of HCC cells, S100A10-KO abolished these effects (FIG. 8C-8D and FIGS. 17I-17L). To evaluate the importance of S100A10 in this process, S100A10 NA or IgG was added together with S100A10 EVs to the lower chambers. The results showed that S100A10 NA completely abolished such promoting effect (FIG. 8E-8F). These findings indicate that S100A10 in EVs is a critical functional component in the chemotaxis of HCC cells.

To this end, we explored the potential receptor of S100A10. As S100A10 was found to increase the activation of EGFR, and EGFR is an important receptor in chemotactic responses,^{33 34} we queried if EGFR is a receptor for S100A10. When we applied EGFR NA to PLC cells in the migration assay, the migratory ability was significantly decreased. Furthermore, EGFR NA treat-ment abolished the differences between S100A10 EVs treated with S100A10 NA and IgG (FIG. 8G). On other hand, EGFR abolished the suppression of cell migration exerted by S100A10 NA (FIG. 8H). Furthermore, coimmunoprecipitation assay showed that S100A10 bound to EGFR, which further supports that EGFR is a potential receptor in S100A10 chemoattracting process (FIG. 8I).

### DISCUSSION

The identification and characterization of specific gene expression signatures and molecular phenotypes of liver progenitor cells in the process of HCC development allows a better under-standing of the molecular pathogenesis and progression of HCC. In the present study, we investigated S100A10 as it was highly expressed in the liver progenitor stage.^{1 2 35} Here our results showed that S100A10 expression was highly upregulated in patients with HCCs and was significantly associated with more aggressive tumour behaviour including more frequent vascular invasion, poorer cellular differentiation and poorer prognosis. S100A10 also promoted stemness properties of HCC, including self-renewal, tumorigenicity, tumour initiation and chemoresistance. S100A10 enhanced HCC metastasis through the EMT process. These findings indicate that S100A10 has a critical role in HCC progression.

Although the extracellular function of S100 family proteins at large has been reported to promote disease progression in cancers, 12-14 there have been few studies investigating the extra-cellular roles of S100A10. To the best of our knowledge, there are no reports on whether S100A10 are secreted into EVs in cancers. In this study, we clearly demonstrated that S100A10 was secreted into EVs, and in this form, it played a key functional role in HCC. EVs derived from S100A10-OE HCC cells promoted the metastasis and stemness features of HCC. In addition, we found that EV-S100A10 promoted the EGFR, AKT and ERK signaling pathways and enhanced the EMT. Of note, all these effects were effectively abrogated with S100A10 NA. Moreover, treatment with S100A10 NA in xenograft mice suppressed HCC growth and enhanced the antitumour efficiency of sorafenib, indicating that S100A10 NA is a potential therapeutic option for HCC treatment. Furthermore, we found that, while EVs could function as a chemoattractant, S100A10 is a key component of EVs in chemoattracting HCC cell migration. Interestingly, S100A10 was able to interact with EGFR and function as a potential ligand of EGFR to activate its signaling pathways and promote chemotaxis of HCC cells.

S100A10 is well accepted as a receptor for plasminogen and plasminogen activator and facilitates the conversion of plasminogen to plasmin, accelerating the degradation of ECM such as fibronectin, active MMPs and growth factors.³⁶⁻³⁹ To this end, combining the results of mass spectrometry, we found that S100A10 increased the MMP2, fibronectin and EGF levels in EVs. Furthermore, we found that MMP2, fibronectin and EGF were limited to the EV surface, while S100A10 localized both within and on the EV membranes. Of note, S100A10 NA was able to inhibit the binding of MMP2, fibronectin and EGF to the surface of EVs. ITGAV has been reported to bind MMP2, fibronectin and EGF.⁴⁰⁻⁴² In addition, it has been reported that targeting of fibronectin to the surface of EVs is dependent on the binding to integrins.²⁹ Along this line, treatment with integrin-binding RGD peptide to inhibit the interaction, we were able to demonstrate that the S100A10-mediated binding of MMP2, EGF and fibronectin to EVs was dependent on the interaction with integrins. Our coimmunoprecipitation results also confirmed that S100A10 physically interacted with ITGAV, MMP2, EGF and fibronectin. All these findings suggest that S100A10 plays a crucial role in mediating targeting of MMP2, EGF and fibronectin to EVs through binding to integrin receptors.

In summary, we demonstrated that S100A10 is secreted into EVs and plays a pivotal role in mediating HCC stemness-related properties and activating EGFR signaling pathway to promote EMT. S100A10 also mediates the targeting of specific secretory proteins to EV membranes through physical interaction with integrins. Moreover, EV-S100A10 also functions as a chemoattractant. Importantly, S100A10 NA is able to block the functions of EV-S100A10, and it may be a potential therapeutic strategy for HCC. Recently, a bioinformatics analysis study revealed the potential usage of S100 protein family as a biomarker for HCC including S100A10.⁴³ As our data also suggest that the EV-S100A10 level was relatively higher in the plasma of patients with HCC than in the plasma of healthy subjects, more work is warranted to test the use of S100A10 or EV-S100A10 as a potential diagnostic marker for HCC.

### SUMMARY

The instant study established that S100A10 was highly expressed in patients' HCC tumors as compared with the corresponding non-tumorous livers. Its upregulation was significantly associated with more aggressive tumor behavior and poorer overall survival rates. Functionally, S100A10 promoted HCC initiation, self-renewal, chemoresistance, and metastasis in vitro and in vivo. Of significance, S100A10 was secreted into the extracellular space and carried in extracellular vesicles (EVs), and the function of S100A10 in EVs was studied for the first time.

EVs from HCC patients exhibited higher S100A10 levels than those from healthy subjects. S100A10-enriched EVs derived from S100A10-overexpressing HCC cells enhanced the sternness and metastatic ability of HCC cells, upregulated AKT and ERK signaling, and promoted epithelial-mesenchymal transition. S100A10 protein in the EVs (abbreviated as EV-S100A10) also functions as a chemoattractant in HCC cell motility. Importantly, it was found that blockage of EV-S100A10 using S100A10-neutralizing antibody significantly abrogated these enhancing effects. Altogether, the results showed that S100A10 promotes HCC progression, significantly via its transfer in EVs. These findings indicate that EV-S100A10 may be a novel biomarker for HCC. It may also be a potential therapeutic target for HCC progression, and neutralizing antibody to S100A10 may a be potential valuable therapeutic strategy for HCC patients.

### REFERENCES

1. Liu M, Yan Q, Sun Y, et al. A hepatocyte differentiation model reveals two subtypes of liver cancer with different oncofetal properties and therapeutic targets. Proc Natl Acad Sci USA 2020; 117:6103-13.
2. Wang X, Wang J, Tsui Y-M, et al. RALYL increases hepatocellular carcinoma stemness by sustaining the mRNA stability of TGF-β2. Nat Commun 2021; 12:1518.
3. Gratias E J, Dome J S, Jennings U, et al. Association of chromosome 1q gain with inferior survival in favorable-histology Wilms tumor: a report from the children's Oncology Group. J Clin Oncol 2016; 34:3189-94.
4. Mirkovic J, Sholl L M, Garcia E, et al. Targeted genomic profiling reveals recurrent KRAS mutations and gain of chromosome 1q in mesonephric carcinomas of the female genital tract. Mod Pathol 2015; 28:1504-14.
5. Bièche I, Champème M H, Lidereau R. Loss and gain of distinct regions of chromosome 1q in primary breast cancer. Clin Cancer Res 1995; 1:123-7.
6. Huang D, Yang Y, Sun J, et al. Annexin A2-S100A10 heterotetramer is upregulated by PML/RARα fusion protein and promotes plasminogen-dependent fibrinolysis and matrix invasion in acute promyelocytic leukemia. Front Med 2017; 11:410-22.
7. Phipps K D, Surette A P, O'Connell P A, et al. Plasminogen receptor S100A10 is essential for the migration of tumor-promoting macrophages into tumor sites. Cancer Res
   2011; 71:6676-83.
8. O'Connell P A, Surette A P, Liwski R S, et al. S100A10 regulates plasminogen-dependent macrophage invasion. Blood 2010; 116:1136-46.
9. Monea S, Lehti K, Keski-Oja J, et al. Plasmin activates pro-matrix metalloproteinase-2 with a membrane-type 1 matrix metalloproteinase-dependent mechanism. J Cell Physiol 2002; 192:160-70.
10. Xiao Y, Zhao H, Tian L, et al. S100A10 is a critical mediator of GAS6/AXL-Induced angiogenesis in renal cell carcinoma. Cancer Res 2019; 79:5758-68.
11. Zhou X, Shi M, Cao J, et al. S100 calcium binding protein A10, a novel oncogene, promotes the proliferation, invasion, and migration of hepatocellular carcinoma. Front Genet 2021; 12:695036.
12. van Lent P L, Grevers L C, Schelbergen R, et al. S100A8 causes a shift toward expression of activatory Fcγ receptors on macrophages via toll-like receptor 4 and regulates Fcγ receptor expression in synovium during chronic experimental arthritis. Arthritis Rheum 2010; 62:3353-64.
13. Prieto D, Sotelo N, Seija N, et al. 5100-A9 protein in exosomes from chronic lymphocytic leukemia cells promotes NF-κB activity during disease progression. Blood 2017; 130:777-88.
14. Wang Y, Yin K, Tian J, et al. Granulocytic myeloid-derived suppressor cells promote the stemness of colorectal cancer cells through exosomal S100A9. Adv Sci 2019; 6:1901278.
15. Noye T M, Lokman N A, Oehler M K, et al. S100A10 and cancer hallmarks: structure, functions, and its emerging role in ovarian cancer. Int J Mol Sci 2018; 19. doi:10.3390/ijms19124122. [Epub ahead of print: 19 Dec. 2018].
16. Balaj L, Lessard R, Dai L, et al. Tumour microvesicles contain retrotransposon elements and amplified oncogene sequences. Nat Commun 2011; 2:180.
17. Thery C, Ostrowski M, Segura E. Membrane vesicles as conveyors of immune responses. Nat Rev Immunol 2009; 9:581-93.
18. Choi D-S, Kim D-K, Kim Y-K, et al. Proteomics, transcriptomics and lipidomics of exosomes and ectosomes. Proteomics 2013; 13:1554-71.
19. Peinado H, Lavotshkin S, Lyden D. The secreted factors responsible for pre-metastatic niche formation: old sayings and new thoughts. Semin Cancer Biol 2011; 21:139-46.
20. Peinado H, Alečković M, Lavotshkin S, et al. Melanoma exosomes educate bone marrow progenitor cells toward a pro-metastatic phenotype through Met. Nat Med 2012; 18:883-91.
21. Schmidt T M, Barwick B G, Joseph N, et al. Gain of chromosome 1q is associated with early progression in multiple myeloma patients treated with lenalidomide, bortezomib, and dexamethasone. Blood Cancer J 2019; 9:94.
22. Lee T K-W, Guan X-Y, Ma S. Cancer stem cells in hepatocellular carcinoma—from origin to clinical implications. Nat Rev Gastroenterol Hepatol 2022; 19:26-44.
23. Tsui Y-M, Chan L-K, Ng I O-L. Cancer stemness in hepatocellular carcinoma: mechanisms and translational potential. Br J Cancer 2020; 122:1428-40.
24. Berasain C. Hepatocellular carcinoma and sorafenib: too many resistance mechanisms? Gut 2013; 62:1674-5.
25. Tovar V, Cornelia H, Moeini A, et al. Tumour initiating cells and IGF/FGF signalling contribute to sorafenib resistance in hepatocellular carcinoma. Gut 2017; 66:530-40.
26. Lu H, Xie Y, Tran L, et al. Chemotherapy-induced S100A10 recruits KDM6A to facilitate OCT4-mediated breast cancer sternness. J Clin Invest 2020; 130:4607-23.
27. Lima L G, Ham S, Shin H, et al. Tumor microenvironmental cytokines bound to cancer exosomes determine uptake by cytokine receptor-expressing cells and biodistribution. Nat Commun 2021; 12:3543.
28. Luo F, Tran A P, Xin L, et al. Modulation of proteoglycan receptor PTP enhances 2018; 9:4126.
29. Sung B H, Ketova T, Hoshino D, et al. Directional cell movement through tissues is controlled by exosome secretion. Nat Commun 2015; 6:7164.
30. Sung B H, Weaver A M. Exosome secretion promotes chemotaxis of cancer cells. Cell Adh Migr 2017; 11:187-95.
31. Chen T, Guo J, Yang M, et al. Chemokine-containing exosomes are released from heat-stressed tumor cells via lipid raft-dependent pathway and act as efficient tumor vaccine. J Immunol 2011; 186:2219-28.
32. Castelli G, Bruno F, Saieva L, et al. Exosome secretion by Leishmania infantum modulate the chemotactic behavior and cytokinic expression creating an environment permissive for early infection. Exp Parasitol 2019; 198:39-45.
33. Bailly M, Wyckoff J, Bouzahzah B, et al. Epidermal growth factor receptor distribution during chemotactic responses. Mol Biol Cell 2000; 11:3873-83.
34. Caric D, Raphael H, Viti J, et al. EGFRs mediate chemotactic migration in the developing telencephalon. Development 2001; 128:4203-16.
35. Yan Q, Zhang Y, Fang X, et al. PGC7 promotes tumor oncogenic dedifferentiation through remodeling DNA methylation pattern for key developmental transcription factors. Cell Death Differ 2021; 28:1955-70.
36. Liu Y-J, Wu D-P, Liang J-Y, et al. Long-term survey of outcome in acute promyelocytic leukemia: a single center experience in 340 patients. Med Oncol 2011; 28 Suppl 1:5513-21.
37. Andreasen P A, Kjøller L, Christensen L, et al. The urokinase-type plasminogen activator system in cancer metastasis: a review. Int J. Cancer 1997; 72:1-22.
38. Taipale J, Koli K, Keski-Oja J. Release of transforming growth factor-beta 1 from the pericellular matrix of cultured fibroblasts and fibrosarcoma cells by plasmin and thrombin. J Biol Chem 1992; 267:25378-84.
39. Falcone D J, McCaffrey T A, Haimovitz-Friedman A, et al. Macrophage and foam cell release of matrix-bound growth factors. Role of plasminogen activation. J Biol Chem 1993; 268:11951-8.
40. Brooks P C, Strömblad S, Sanders L C, et al. Localization of matrix metalloproteinase MMP-2 to the surface of invasive cells by interaction with integrin alpha v beta 3. Cell 1996; 85:683-93.
41. Vial D, McKeown-Longo P J. Epidermal growth factor (EGF) regulates α5β1 integrin activation state in human cancer cell lines through the p90RSK-dependent phosphorylation of filamin a. J Biol Chem 2012; 287:40371-80.
42. Deryugina E I, Ratnikov B, Monosov E, et al. MT1-MMP initiates activation of pro-MMP-2 and integrin alphavbeta3 promotes maturation of MMP-2 in breast carcinoma cells. Exp Cell Res 2001; 263:209-23.
43. Zheng S, Liu L, Xue T, et al. Comprehensive analysis of the prognosis and correlation with immune infiltration of S100 protein family members in hepatocellular carcinoma. Front Genet 2021; 12:648156.

We claim:

1. A method for identifying a subject as having an elevated risk of having hepatocellular carcinoma (HCC), the method comprising:
determining the level of S100A10 in the sample obtained from the subject,
identifying the subject as having HCC if the level of S100A10 in the sample is increased compared to the level of S100A10 in a control sample.

2. The method of claim 1, wherein the sample is a plasma sample or a liver biopsy.

3. The method of claim 1, wherein the control sample is a sample from a healthy subject.

4. The method of claim 1, wherein the level of S100A10 is at least 10% greater than the control.

5. The method of claim 1, wherein the step of determining the level of S100A10 in the sample comprises measuring the level of S100A10 protein in the sample, preferably, the level of S100A10 protein derived from the extracellular vesicles of the sample.

6. The method of any clam 1, wherein the step of determining the level of S100A10 in the sample comprises performing an enzyme-linked immunosorbent assay (ELISA), radioimmunoassay (RIA), western blot, and dot blot, and
wherein the step of determining the level of S100A10 in the sample comprises measuring the level of S100A10 mRNA in the sample.

7. The method of claim 6 further comprising performing a hybridization assay, Real-time Polymerase chain reaction (RT-PCR), or Quantitative Polymerase chain reaction (qPCR).

8. The method of claim 1, wherein the step of identifying the subject as having HCC is such identification with at least a 50%, 60%, 70%, 80%, 90%, 95%, 97%, or 99% certainty.

9. The method of claim 1, further comprising the step of treating the identified subject by administering a therapeutic agent for treating HCC,
wherein the therapeutic agent is selected from the group consisting of antibodies, small molecule inhibitors, and inhibitory nucleic acids.

10. The method of claim 9, wherein the small molecules are effective in inhibiting the S100A10-annexin A2 interaction.

11. The method of claim 9, wherein the antibodies are neutralizing antibodies against annexin A2 and/or S100A10.

12. The method of claim 9, wherein the inhibitory nucleic acids are shRNA targeting S100A10.

13. A method of treating a subject having hepatocellular carcinoma (HCC), the method comprising:
determining the level of S100A10 in a sample obtained from the subject, administering to the subject an effective amount of a therapeutic agent for treating HCC if the level of S100A10 in the sample is increased compared to the level of S100A10 in a control sample.

14. The method of claim 13, wherein the level of S100A10 is at least 10%, 20%, 30%, 40%, 50%, 60%, 70%, 80%, 90%, 100%, or more than 100% greater than the control.

15. The method of claim 13, wherein the therapeutic agent is selected from the group consisting of antibodies, small molecule inhibitors, and inhibitory nucleic acids.

16. The method of claim 13, wherein the small molecules are effective in inhibiting the S100A10-annexin A2 interaction.

17. The method of claim 13, wherein the antibodies are neutralizing antibodies against annexin A2 and/or S100A10.

18. The method of claim 13, wherein the inhibitory nucleic acids are shRNA targeting S100A10.

19. The method of claim 13, wherein the therapeutic agent is administered in an amount effective to reduce or inhibit activities and/or quantities of S100A10.

20. The method of claim 13, wherein the therapeutic agent is administered in an amount effective to reduce or inhibit the quantity of extracellular vesicles expressing S100A10 in the blood of the subject.

21. The method of claim 13, wherein the therapeutic agent is administered in an amount effective to reduce or inhibit proliferation, migration, invasion, motility, and/or metastatic abilities of the cancer cells of HCC.

22. The method of claim 13, wherein the therapeutic agent is administered in an amount effective to reduce or inhibit tumor growth, tumor burden, and/or increase survival of the subject.

23. The method of claim 13, wherein the therapeutic agent is administered in an amount effective to reduce or inhibit the number of cancer stem cells (CSCs) in HCC, or the number of the cancer cells expressing one or more of CD24, CD44, LGR5, SOX2, c-MYC, CD133, EpCAM, and ABCG2, or the level of expression of one or more of CD24, CD44, LGR5, SOX2, c-MYC, CD133, EpCAM, and ABCG2 on the cancer cells.

24. The method of claim 13, wherein the therapeutic agent is administered in an amount effective to reduce or inhibit activities and/or quantities of one or more kinases of EGFR, AKT and ERK signaling associated with epithelial-mesenchymal transition of the cancer cells.
